(19) United States
(12) Patent Application Publication
(10) Pub. No.: US 2023/0039637 A1
(43) Pub. Date: Feb. 9, 2023

(54) MICROBIOME OPTIMIZATION

(71) Applicant: GLYCIOME, LLC, Valleyford, WA (US)

(72) Inventors: Joanna E. ELLINGTON, Valleyford, WA (US); G. D. CLIFTON, Valleyford, WA (US); Rayne ELLINGTON-LAWRENCE, Valleyford, WA (US)

(73) Assignee: GLYCIOME, LLC, Valleyford, WA (US)

(21) Appl. No.: 17/788,674

(22) PCT Filed: Dec. 19, 2020
(86) PCT No.: PCT/US20/66269

§ 371 Date: Jun. 23, 2022

Related U.S. Application Data
(63) Continuation-in-part of application No. 16/943,895, filed on Jul. 30, 2020.
(60) Provisional application No. 62/953,068, filed on Dec. 23, 2019.
(60) Provisional application No. 63/005,243, filed on Apr. 4, 2020.
(60) Provisional application No. 63/075,132, filed on Sep. 5, 2020.

(51) Int. Cl. A61K 31/045; A61K 31/22; A61K 47/26; A61K 9/00; A61P 15/00; G01N 33/569; G01N 27/02; G16H 10/40

(52) U.S. Cl. CPC A61K 31/045; A61K 31/22; A61K 47/26; A61K 9/0034; A61P 15/00; G01N 33/56911; G01N 27/02; G16H 10/40

(57) ABSTRACT

The present disclosure provides compositions and methods for acidic compositions for use in optimizing the genital microbiome of a user or sexual partners of that user. The compositions may comprise a prebiotic oligosaccharide, a metal co-factor, and an essential oil comprising bornyl acetate. The compositions support the genital microbiota and are useful for, for example, hydrating, lubricating, cleaning, and/or decreasing irritation or inflammation of the urogenital and/or anogenital region of a subject, and/or enhancing the beneficial genital microbiota of a subject. Such compositions are useful before, during, and/or after sexual and/or reproductive activity. Furthermore, the compositions may have minimal or beneficial effect on gametes.

### CROSS REFERENCE TO RELATED APPLICATIONS

The present application claims priority to U.S. App. No. 63/953,068, filed Dec. 23, 2019, U.S. App. No. 63/005,243, filed Apr. 4, 2020, U.S. application Ser. No. 16/943,895, filed Jul. 30, 2020, and U.S. App. No. 63/075,132, filed Sep. 5, 2020, each of which are hereby incorporated by reference in their entirety.

### BACKGROUND

The genital microbiome of a human is a unique combination of microbial species comprising at least one hundred species of bacteria and a variety of fungal, viral, and protozoal species. There is considerable variation in make-up of the genital microbiome between individuals, with many factors such as hygiene regimes, diet, environment, age, ethnicity, disease, sexual activity, sexual orientation, and life history affecting the presence of specific microbial species and their metabolic activities. Environmental conditions within the genitalia compared to other locations of the human body are distinct. As a result, products indicated for urogenital, anogenital, vaginal and/or penile use, to modulate the genital microbiome, may require distinct prebiotic and/or probiotic constituents.

Additionally, the genital microbiome is unique in that it is shared between members of a sexual dyad and between mammalian mother and newborn as a primary mode of the newborn's establishment of its own microbiome. Diseases and/or dysbiosis within the dyad can occur if a healthy genital microbiome is not supported. Microbiome congruency and transfer of microbial species between sexual partners, and between a mother and her child is well documented. Sexual partners can transmit pathogenic bacteria back and forth that harm beneficial bacteria, increase inflammation, and increase risk for disease, including infertility, reproductive dysfunction, poor pregnancy outcomes, autoimmune disease, sexually transmitted diseases (e.g., HIV, HSV, HPV), cancers (e.g., prostate, cervical), and systemic diseases such as cognitive impairment.

In particular, bacterial vaginosis (BV) is a common bacterial imbalance of the vaginal microbiome. As the genital microbiome is often shared between sexual partners, the penis often has an effect on the bacterial balance of the vagina. For example, correlations between BV incidence and penile microbiota have been shown which suggest the exchange of By-associated bacteria between sexual partners through intercourse. Furthermore, male genital dysbiosis (e.g., penile growth of the same organisms that cause BV) may be etiologically connected with various sexually transmitted diseases such as HIV, HSV, or HPV, urethritis, abnormal sperm quality, and penile cancers.

Existing cleaning products and genital therapies (e.g., body washes, wipes, yeast treatments, most lubricants) have pH, salt levels and ingredients that harm genital tissues, gametes, and kill healthy bacteria. For example, a popular, commercially available diaper wipe has a pH of 3 while the World Health Organization (WHO) has stated that this pH level is inconsistent with human genital tissues. Furthermore, measurement driven application regimens are often lacking and current modalities are insufficient. For example, over the counter (OTC) pH tests are usually nitrazine paper-based, with color scales that are difficult or impossible (16% of the time) to match to manufacturer's scale, depend on lighting and eyesight, only have moderate agreement between users and change if moved from the physiologic environment (vagina).

There remains a need for compositions for use in the urogenital and/or anogenital region that are not harmful to the genital tissues and that preserve and support the beneficial regional microbiome of these areas. Presently disclosed embodiments address this need and provide other related advantages.

### SUMMARY

The present disclosure provides compositions and methods for maintaining and/or optimizing the microbiome of certain subject bodily regions including the urogenital (e.g., subject regions relating to function of urinary excretion and reproduction) and/or anogenital regions (e.g., relating to the anus and genitals). These methods may involve measurements of the metabolomic profile and/or pH of these bodily regions in an attempt to inform the treatment regimen (e.g., when to administer, which type of composition to deliver, etc.) In particular, the present disclosure relates to isotonic, genital microbiome-friendly topical compositions having an acidic pH. Without wishing to be bound by theory, it is believed that the application of these acidic pH to the male genitals has a minimal (e.g., within 20% of a measured parameter) or beneficial effect on gametes. These compositions may comprise a prebiotic oligosaccharide, a metal co-factor, and borneol, or pharmaceutically acceptable salts or prodrugs thereof such as bornyl acetate (e.g., via an essential oil comprising bornyl acetate), at an acidic pH level (e.g., less than 7 or less than 6.5 or less than 6 or less than 5.5 or less than 5 or from 4 to 6 or from 4 to 5 or from 4.25 to 4.75). The isotonic, genital microbiome-friendly compositions of the present disclosure can be used for enhancing the genital microbiota of a subject (e.g., increasing the colony count of beneficial genital microbiota of a subject), enhancing gamete function of a subject, reducing sexually transmitted disease incidence, penile cancer persistence or occurrence, or urethritis, and/or increasing or maintaining sperm quality. In another aspect, the disclosure herein provides an integrated penile pH tracking system for the measurement of penile pH. The present disclosure also provides a relatively non-invasive intervention treatment to facilitate clearance of an STD, such as HPV or HIV or other viral infection thereby reducing sexually transmitted disease persistence, which may otherwise lead to penile and other cancers. Furthermore, these methods may also decrease the occurrence of bacterial vaginosis in a sexual partner of the subject having applied the compositions disclosed herein.

In some embodiments, the method of optimizing the beneficial microbiome growth in the genital region of a subject in need thereof may comprise application of a pharmaceutical composition having an acidic pH to the genital region of the subject or to the genital region of a sexual partner of the subject in need thereof.

Also provided are systems for the measurement of one or more parameters of the metabolomic profile of a biological sample (e.g., derived from saliva, blood, urine, feces, genital fluids, tears, nasal swabs, sweat, psoriasis lesions). These systems may be used in concert with the methods described herein. In some embodiments, the system may comprise:
a substrate;
a sensor medium immobilized on said substrate comprising a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile of the biological sample.

These systems may be used in devices for measurements of the metabolomic profile and/or pH of biological samples. In some embodiments, the device for the measurement of one or more parameters of the metabolomic profile of a biological sample (e.g., derived from saliva, blood, urine, feces, genital fluids, tears, nasal swabs, sweat, psoriasis lesions) may comprise:
a) a handle portion dimensioned to be held in a user's hand comprising a power source (e.g., one or more batteries such as a lithium ion battery, one or more solar cells); and
b) a sensor portion comprising one or more sensors; wherein the sensors comprise:
   a substrate;
   a sensor medium immobilized on said substrate comprising a plurality of carbon nanostructures; wherein the plurality of carbon nanostructures has one or more conductive materials deposited thereon;
   at least two conductive terminals in electrical connection with the sensor medium and spaced from each other;
wherein said sensor portion is removably attached to the handle portion; and when the sensor portion is attached to the handle portion, the one or more systems are in electrical communication with the power source;
and said device comprises at least one measurement system to measure one or more electrical properties of the sensor medium when the sensor medium comprises the biological sample deposited thereon.

Methods for the measurement of one or more parameters of the metabolomic profile of a biological sample (e.g., derived from saliva, blood, urine, feces, genital fluids, tears, nasal swabs, sweat, psoriasis lesions) are also provided which may comprise:
depositing the biological sample on a sensor medium comprising a plurality of carbon nanostructures; wherein the plurality of carbon nanostructures has one or more conductive materials deposited thereon;
measuring an electrical property of the sensor medium after depositing the biological sample; and
correlating the measured electrical property to the one or more parameters of the metabolomic profile.

These systems, devices, and methods of measurement may also be used with administration regimens. For example, some embodiments for the method of optimizing the beneficial microbiome growth in the genital region of a subject in need thereof may comprise:
measurement of one or more parameters of the metabolomic profile of a biological sample with the presently disclosed measurement systems and/or devices and/or methods;
application of a pharmaceutical composition having an acidic pH to the genital region of the subject or to the genital region of a sexual partner of the subject in need thereof based on the one or more parameters of the metabolomic profile.

Kits which may be used with the compositions as described herein are also disclosed. The Kit may comprise an upper waste receptacle, and a lower portion comprising one or more dispensing compartments;

wherein the kit is configured to hold a supply of applicators for treatment with a gel composition (e.g., a gel composition comprising bornyl acetate, a metallic cofactor, and a prebiotic oligosaccharide buffered as described herein) of about one month or greater;
wherein said waste receptacle is configured to hold or safely contain waste products from said treatment; and
wherein said kit is composed of one or more degradable materials that can be safely disposed of by burning, incineration, and/or recycling.

In some embodiments, the compositions (e.g., topical, topical isotonic, gels, lubricants) of the present disclosure comprise:

(a) a metallic co-factor (e.g., manganese chloride);

(b) a prebiotic oligosaccharide (e.g., lactulose); and

(c) borneol or a prodrug thereof (e.g., bornyl acetate);

wherein the composition is buffered with a buffer system comprising gluconolactone. In particular, the compositions provide an increased buffering capacity in a pH range to support beneficial microbiome optimization for a person in need thereof (e.g., in the vaginal microbiome). The compositions may, for example, have increased buffering capacity in a pH range of from 4-7 or from 5-7 or from 5-6.

### BRIEF DESCRIPTION OF THE FIGURES

FIG. 1 depicts an applicator of the present disclosure which may be used for delivery of compositions to the urogenital and/or anogenital region (e.g., vagina).

FIG. 2 depicts the use of the applicator shown in FIG. 1.

FIG. 3A depicts an applicator of the present disclosure in storage configuration. FIG. 3B depicts the applicator in FIG. 8A nearly in the application configuration. FIG. 3C depicts the applicator in the disposal configuration.

FIG. 4A depicts an applicator of the present disclosure disassembled (e.g., as it may come in a kit). FIG. 4B depicts the applicator of FIG. 4A being assembled.

FIG. 5 shows an exemplary design of a kit described herein.

FIG. 6 shows the average pH values of various regions of the penis (n=6) with a forearm (ante fossa) control. Error bars illustrate the standard deviation.

FIG. 7 shows the average pH values of various regions of the penis with a forearm (ante fossa) control following administration of a foaming wash composition of the disclosure (Formulation A) and a conditioner of the disclosure (Formulation B). For comparison, pH values are also illustrated for a commercial Nivea wash followed by Astroglide conditioner. Error bars illustrate the standard deviation for each measurement.

FIG. 8A is a comparison between the percent development of mouse embryos exposed to an acidic composition of the present disclosure as compared to control. FIG. 8B is a comparison of mouse oocytes fertilized and embryos developed following 4 hr oocyte and sperm exposure to an acidic composition of the present disclosure as compared to control.

FIG. 9A shows the percent total motility of sperm as compared to control measured in the bovine sperm motility studies. FIG. 9B shows the percent of progressively motile sperm compared to control in the bovine sperm motility studies. Formulations tested were 71519A, 71519B, 71519C, 0618CA45, and 0618CA68 (Table 39).

FIG. 10A shows the percent total motility of sperm as compared to control measured in the bovine sperm motility studies. FIG. 10B shows the percent of progressively motile sperm compared to control in the bovine sperm motility studies. Formulations tested were 060919C, 0617HF, 0617HL, and 0617HM (Table 40).

FIG. 11 shows the measured sperm motility as a percent of control for preservative free formulations at pH 4.5 (black bar), preservative free formulations at pH 6.8 (white bar), compositions comprising sodium benzoate preservative at pH 4.5 (dotted bar), and compositions comprising sodium benzoate at pH 6.8 (diagonally stripped bar).

FIG. 12 shows the measured colony forming units of colony forming units found in a formulation comprising a preservative.

FIG. 13 illustrates the changes in progressive motility of human sperm following application of preservative free compositions of the present disclosure of various pH as compared to control.

FIG. 14 illustrates the changes in progressive motility of human sperm following application of a preservative free composition and compositions containing different concentrations of preservatives as compared to control.

FIG. 15 illustrates shows the measured colony forming units of colony forming units found in a formulation measured from the preservative screening test.

FIG. 16 shows the L. crispatus CFU recovery at time 0, 4 h and 24 h after mixing with the test products applied undiluted 1:1. Data represent 6 individual experiments.

FIG. 17 compares the Lactobacillus CFU recovery at time 0 (top), 4 (middle), and 24 (bottom) hrs as compared to control at four different repeat experiments.

FIG. 18 shows the L. crispatus CFU recovery at time 0, 4 h and 24 h after applying a serial dilution of Formula H in the bacterial culture broth. Data represent 6 individual experiments mixing gel with broth 1:1 (100% Formula H) and one experiment with serial dilution (50, 25 and 12.5% by volume).

FIG. 19 shows the L. crispatus CFU recovery at time 0, 4 h and 24 h after applying a serial dilution of a foaming cleanser or conditioning moisturizer in the bacterial culture broth. Data represent 6 individual experiments of growing the bacteria in the culture broth (0% test product) setting the baseline optimal growth and two experiments mixing gel with broth 1:1 with serially diluted gel preparations (100, 50, and 25% by volume).

FIG. 20 shows the number of CFU of P. bivia as compared to L. crispatus after 24 hours of application to an in vitro model.

FIG. 21 illustrates a device of the present disclosure wherein the measurement system is illustrated. The device is illustrated in several configurations demarcated 1-4. Configuration 1 shows a stored configuration of the sensor array (not shown) and with an applicator handle (circular features) which comprises a protective covering. Configuration 2 shows the circular applicator handle to the sensing medium. In this configuration, the biological sample may be collected, for example, by placement of the sending medium on the portion of the body to be measured such as a mucus membrane, skin, penis, or vagina or in contact with a biological fluid such as a genital fluid (e.g., urine). In some embodiments, the sensing medium is a disposable sheath placed over the rectangular dipstick. Configuration 3 illustrates the measurement handle portion of the device, without the sensing medium attached. The sensing medium having the biological sample deposited thereon (or in contact in situ) may be inserted into the measurement handle (Configuration 4). The assembled device may transmit information collected from the sensing medium to an external device such as a smart phone device. The smart device, after receiving the information, displays the pH of the biological sample and interprets the appropriate treatment from measurement.

FIG. 22 shows the mucoadhesion of porcine mucus admixed with a lubricant composition of the disclosure (Table 49) as compared to commercial RepHresh™ and Trimo-san® lubricants, and Summer's Eve® douche. Error bars represent the standard deviation.

FIG. 23 shows a comparison of the lubricity or slipperiness of RepHresh™ Formulation PL 1206 (also known as PL 102345 or 1004BA10), and Gun Oil® at 15 seconds and 60 seconds.

FIG. 24 shows a comparison of the tackiness of RepHresh™, Formulation PL 1206 (also known as PL 102345 or 1004BA10), and Gun Oil®.

FIG. 25 shows the bactericidal effects of Formulation 061819C on Lactobacillus crispatus compared to no gel at 0 time, 4 hours, and 24 hours.

FIG. 26 shows the results of bacterial growth of Lactobacillus crispatus mixes with serially diluted Formulation 061819C performed using a microplate ATP assay at time 0, 4 hours, and 24 hours.

FIG. 27 illustrates measurements of the vaginal pH before and after coital ejaculation, indicating baseline pH<4.5, elevation immediately after coitus to ˜7 (best for sperm survival), and return to baseline within 12 hrs (in vivo data).

FIG. 28A shows the pH titration curve of vaginal fluid simulant and FIG. 28B shows the pH titration curve of vaginal fluid simulant with a lubricant of the present disclosure (“Glyciome Prebiotic Lube”). FIG. 28C provides a comparison of these measurements. Error bars represent standard deviation and “*” represents p<0.05 of the Glyciome Prebiotic Lubricant in combination with Vaginal Fluid Simulant (VFS) as compared to VFS alone.

FIG. 29 shows measured Anterior Vaginal pH before and after coitus using glass-electrode pH meter in single total-hysterectomized woman on Hormone Replacement Therapy (HRT) (e.g., no cyclic hormone changes) (2-4 replicates/trt) measured over time until return to healthy vaginal pH (<4.5). The dotted bar illustrates pH of 4.5. Measurements at 36 hours post coitus were not performed for Glyciome treatments as healthy vaginal pH had already been reestablished.

FIGS. 30A and 30B illustrate the effect of gel bases as compared to gels comprising a combination of lactulose, manganese, and bornyl acetate on vaginal fluid simulant buffering capacity.

FIG. 31 shows the effect of lubricant gels on the present disclosure (GFG) on vaginal fluid simulant (VFS), semen fluid simulant (SFS), and combinations thereof.

FIG. 32 compares the effects of lubricant gels of the present disclosure on vaginal fluid simulant and semen fluid simulant mixtures as compared to a known fertility lubricant (Pre-Seed).

### DETAILED DESCRIPTION

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. Any concentration ranges recited herein are to be understood to include concentrations of any integer within the range and fractions thereof, such as one tenth, one hundredth, and one thousandth of an integer, unless otherwise indicated. Unless otherwise indicated, it will be understood that any percentage refers to the weight percentage with respect to the indicated component. Typically, the percent of a component in the composition indicates the weight percentage with respect to the weight of the composition.

The term “consisting essentially of” is not equivalent to “comprising” and refers to the specified materials or steps, or to those that do not materially affect the basic and novel characteristics of the claimed subject matter. It should be understood that the terms “a” and “an” as used herein refer to “one or more” of the enumerated components.

A dyad is often a group of two persons having a sociologically significant relationship (e.g., sexual relationship, parent-infant relationship, parent-child relationship, health care provider and patient relationship, care-giver and patient relationship). Application of the compositions described herein may optimize or maintain the microbiome of one or more members of a sexual relationship including a sexual dyad. The dyad may be composed of two males, one male and one female, or other gender groupings (e.g. non-binary gendered individual, intersex individual). In certain embodiments, a dyad may be a sexual dyad such heterosexual dyad, a homosexual dyad, or other sexual orientation dyad. In other embodiments, the sexual relationship may be non-binary such as a sexual triad (a group of three people).

The genital microbiota or genital microbiome may include the collective microorganisms that normally colonize the genital region. The genital microbiota may be non-pathogenic. The genital microbiota may refer to that of the genital skin microbiota, the vaginal microbiota (e.g., vaginal mucosal microbiota) of a female subject, the cervical microbiota of a female subject, penile microbiota (e.g., penile skin microbiota such as the foreskin microbiota or the urethral meatus microbiota) of a male subject, microbiota of the genital tissue of an intersex individual, microbiota of a non-binary gendered individual, or any combination thereof. Recently, species overlap between rectal and urinary microbiome species in an individual have been observed. For example, the bacteria of the genital region have been found to represent a continuum between organs of excretion and reproduction as discussed Y. Govender, et al., Front Cell Infect Microbiol 9 (2019): 133, hereby incorporated by reference in its entirety and particularly in relation to the urinary microbiomes disclosed therein. These bacteria of the genital region are referred to herein as the anogenital and/or urogenital microbiomes.

Genital probiotic bacteria may refer to live bacteria, which when administered in adequate amounts to the vagina or penis confer a health benefit (e.g., such as those described herein) to the host subject.

The vaginal microbiota or vaginal flora refers to the collective microorganisms that normally colonize the vulva, clitoris, vestibule, and vagina and are non-pathogenic. In general, beneficial vaginal microbiota is primarily comprised of different strains of Lactobacillus (or related acid-producing bacterial types), which produce lactic acid to keep the vaginal ecosystem at a tightly controlled acidic environment (e.g., pH of 3.5-5.5 or 3.5-5) during much of a woman's monthly cycle in reproductive aged women. An exemplary vaginal microbiome includes a dominance of healthy Lactobacillus species including: Lactobacillus jensenii, Lactobacillus gasseri, and Lactobacillus crispatus. Other beneficial species may include Lactobacillus acidophilus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobaccillus reuteri, and Lactobacillus rhamnosus. Additional bacterial species often present in lower numbers in the vaginal region of healthy women include: Lactobacillus iners, and species from the genuses Prevotella, Megasphaera, Colstridium, Baccilus, Gardnerella, Sneathia, and Mycoplasma. Additionally, acid producing bacteria that may be part of the normal vaginal microbiota in some women including Lactobacillus iners, and species of Prevotella, Atopobium, Leptotrichia, Leuconostoc, Megasphaera, Pediococcus, Streptococcus, and Weissella. The species found in normal vaginal microbiota can vary depending on age and ethnicity. In some ethnicities and life stages, the acid-producing, healthy vaginal microbiome may not be dominated by Lactobacillus spp. Evidence has shown that Lactobacillus spp. dominance is associated with optimal reproductive and genital health outcomes in women of all ages. Various microbiota (and their connection with both vaginal and non-vaginal health) are disclosed in J. Si, et al., Cell Host & Microbe 21 (2017): 97-105, hereby incorporated by reference in its entirety, and particularly in relation to vaginal microbiota including Lactobacillus and Prevotella species.

Reproductive tract microbiota may include the collective microorganisms that normally colonize the female upper reproductive tract, such as the cervix, uterus, Fallopian tubes, and ovaries, and are non-pathogenic. In certain embodiments, the female reproductive tract microbiota can also be comprised of species from the genus Pseudomonas, Acinetobacter, Vagococcus, Sphingobium, Erysipelothrix, Facklamia or Prevotella. In certain embodiments, application of the present compositions may have differential effect on species in the reproductive tract microbiota. For example, application of the present compounds may result in increases or maintained levels on beneficial bacteria such as healthy Lactobacillus species such as L. crispatus, L. jensenii, and L. gasseri. In some embodiments, administration of the compositions of the present disclosure may inhibit the growth of pathobionts such as those from the Gardnerella genus (e.g., G. vaginalis) or the Prevotella genus (e.g., P. bivia). In some embodiments, administration may result in increases or maintained levels on beneficial bacteria such as healthy Lactobacillus species such as L. crispatus, L. jensenii, and L. gasseri and inhibition of the growth of pathobionts such as those from the Gardnerella genus (e.g., G. vaginalis) or the Prevotella genus (e.g., P. bivia) for example, as measured between 12 and 48 hours following administration. In some embodiments, administration of the present compositions may result in increases or maintained levels on beneficial bacteria such as healthy Lactobacillus species such as L. crispatus, L. jensenii, and L. gasseri and reduction of pathobionts in the microbiome including one or more pathobionts such as those from the Gardnerella genus (e.g., G. vaginalis) or the Prevotella genus (e.g., P. bivia) for example, as measured between 12 and 48 hours following administration.

The vaginal microbiota is often affected by the penile microbiota and correlations between BV and penile microbiota between members of a sexual dyad have been shown in C. Liu, et al., mBio 6 (2015): e00589-15, hereby incorporated by reference in its entirety and particularly in relation to the exchange of BV associated bacteria through intercourse and connections between Nugent score and penile community state types. Penile microbiota or penile flora may refer to the collective microorganisms that normally colonize the penis, foreskin, and distal urethra which are non-pathogenic. The penis includes the penile shaft and distal glans, which includes the glans, glans coronal, meatus urethralis, fossa navicularis, frenulum, coronal sulcus, and foreskin. In various implementations, the penile microbiota, and in particular, in an optimized penile microbiome, comprises bacterial species from the genus Lactobacillus, Streptococcus, Staphylococcus, Corynebacteria, and combinations thereof. In certain embodiments, the penile microbiota comprises Lactobacillus crispatus, Lactobacillus jensenii, Lactobacillus iners, Lactobacillus gasseri, Streptococcus, non-pathogenic Prevotella, Corynebacteria, Staphylococcus, Anaerococcus, Peptoniphilus, Finegoldia, Porphyromonas, Propionibacterium, Delftia, Bifidobacterium, Clostridium, non-pathogenic Pseudomonas, or any combination thereof. In certain embodiments, the penile microbiota of a male subject reflects the vaginal microbiota and/or reproductive tract microbiota of a female subject, wherein the male subject and female subject are members of a sexual dyad. The species found in normal penile microbiota can differ between circumcised and uncircumcised subjects. For example, penile microbiota may also include sperm microbiota such as those disclosed in D Baud, et al., Frontiers in Microbiol 10 (2019): 234, hereby incorporated by reference in its entirety and particularly in relation to beneficial seminal microbiota including Lactobacillus species. In some embodiments, the compositions are able to increase the number of beneficial species in the penile microbiome (and by extension the vaginal microbiome following intercourse) such as Lactobacillus, Streptococcus, Staphylococcus, Corynebacteria, and optimize or balance (e.g., decrease and/or increase) the number of dysbiosis associated anaerobes such as those from the species Prevotella, Finegoldia, Diallister, Snethia, Megasphaeae, Mobiluncus, Mycoplasma, Peptococcus, Peptostreptococcus, Porphyromonas, Slackia, Tannerella, Treponema, Ureaplasma, Veillonella, Actinobacteria, Anaerococcus, Actinomyces, Aggregatibacter, Atopobium, Bacteroides, Bifidobacteriium, Clostridiales, Eggerthella, Eubacterium, Fusobacterium, Garnderella, Leptotrichia, and combinations thereof. In some embodiments, optimization of the microbiome involves decreasing the number of pathogenic communities of bacteria such as those species from the genera Gardnerella, Finegoldia, Dialister, Prevotella, Anaerococcus, Atopobium, Megasphaera, and combinations thereof.

The anogenital region of a subject includes regions of the anus and the genitalia. In certain embodiments, the female anogenital region comprises the cervix, vagina, vulva, clitoris, urethral meatus, urethral meatus, vulval vestibule, perineum, and/or anus. In certain embodiments, the male anogenital region comprises the penis, base of the penis, foreskin, urethral meatus, scrotum, perineum, and anus. The term urogenital region may refer to the region of the distal urinary tract and the genitalia. In certain embodiments, the female urogenital region comprises the cervix, vagina, vulva, clitoris, introitus, urethral meatus, urethral fold, vulval vestibule, and/or perineum. In some subjects, the anogenital and/or urogenital regions of a subject may be indistinct, intersex, or transitioning from male to female or female to male due to iatrogenic (e.g., surgery or hormone therapy) or natural/genetic causes.

Genital tissues are often living cells found in the anogenital and/or urogenital regions. Genital tissues include, but are not limited to epithelial surface cells (e.g., skin), mucosal cells, immune cells, nerve cells, blood cells, connective tissue cells, and neoplastic cells of the vulva, clitoris, vagina, vestibule, vulval vestibule, urethral meatus, penis, foreskin, distal urethra, and scrotum. Since sperm cells exit the penis and are often deposited on genital tissue (e.g., vagina), genital tissues also include semen and sperm cells.

Genital fluids are often secretions from the body that naturally occur in and around genital tissues. Genital fluids include, but are not limited to, cervical and vaginal secretions (together often referred to as cervico-vaginal fluids (CVF)), semen, smegma, seminal fluid, urethral secretions, epithelial and mucosal coatings, menses flow, post-partum lochia, amniotic fluid, and other fluids naturally occurring in and around the vagina, vulva, clitoris, penis, foreskin, and scrotum. Gametes may be found in genital fluids. For example, male gametes may be found in male genital fluids such as semen, smegma, seminal fluid, and/or urethral secretions or fluid.

The reproductive cycle or menstrual cycle is a cycle of hormone changes comprising both the production of an oocyte (ovarian cycle) and preparation of the uterus for pregnancy (uterine cycle). A cycle of the reproductive cycle may include the time of peak fertility in the cycle immediately before and after ovulation as well as the period when conception is not possible due to the lack of a viable egg.

“Effective amount” or “therapeutically effective amount” refers to that amount of a composition of this disclosure which, when administered to a subject, such as a human, is sufficient to affect a desired biological effect or treatment including optimizing of the penile microbiome. In some embodiments, the effective amount may have minimal effect on gametes following application (e.g., with minimal changes in motility, concentration, vitality, morphology of gametes, oxidation-reduction potential, sperm DNA fragmentation, sperm mitochondrial membrane potential, survival, and changes at the sub-cellular levels such as changes to proteins related to specific functions of the gametes). In some embodiments, the therapeutically effective amount alters one or more features of gametes in the genital fluids of the user by less than 20%.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans, etc.). A subject in need thereof is typically a subject for whom it is desirable to treat a disease, disorder, or condition as described herein (and in particular, treatment of a disease, disorder or condition relating to dysbiosis of the urogenital and/or anogenital regions). For example, a subject in need thereof may seek or be in need of treatment, require treatment, be receiving treatment, may be receiving treatment in the future, or a human or animal that is under care by a trained professional for a particular disease, disorder, or condition.

As used herein, the phrase “pharmaceutically acceptable” generally safe for ingestion or contact with biologic tissues at the levels employed. Pharmaceutically acceptable is used interchangeably with physiologically compatible.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecyl sulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Prodrugs are typically compounds that may be converted under physiological conditions or by solvolysis to a biologically active compound of the invention. Prodrug may refer to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the indicated compound, for example, by hydrolysis in blood. The prodrug compound often offers advantages of solubility, tissue compatibility or delayed release in a mammalian organism as described in Bundgard, H., Design of Prodrugs (1985): 7-9, 21-24 (Elsevier, Amsterdam), Higuchi, T., et al., ACS Symposium Series, Vol. 14, and Bioreversible Carriers in Drug Design, Ed. Edward B. Roche, American Pharmaceutical Association and Pergamon Press, 1987, each of which are hereby incorporated by reference in their entirety.

The present disclosure provides compositions (e.g., acidic compositions, topical isotonic compositions, acidic topical isotonic compositions) which may comprise a prebiotic oligosaccharide, a metal co-factor, and bornyl acetate (e.g., the composition comprises an essential oil comprising bornyl acetate). Such compositions have no or minimal irritation to the epithelial tissues of the urogenital and/or anogenital regions; no or minimal disruption of natural mucus secretions and mucins of the urogenital and/or anogenital regions; minimal or no inhibition on the normal, non-pathogenic microbiota of the urogenital and/or anogenital regions; do not promote growth of pathogenic bacteria of the urogenital and/or anogenital regions; limit transfer of pathogenic bacteria from a first member of a dyad to a second member of a dyad; enhance transfer of beneficial microbiota species from a first member of a dyad to a second member of a dyad; or any combination thereof.

Distinct genital microbiome states and metabolic profiles have been identified that correlate with health and conversely with genital disease (e.g., penile or vaginal disease), fertility, poor birthing outcomes, HIV susceptibility, HPV susceptibility, sexually transmitted infections, autoimmune disease, and cancer risk. Systemic risk diseases such as cognitive impairment/dementia may also be associated with the chronic inflammation and dysbiosis of the genital microbiome as shown in R. Muzambi, et al., BMJ Open 9 (2019): e030874, hereby incorporated by reference in its entirety, and particularly in relation to the connection between bacterial infections and dementia. Additionally, diseases such as Type 1 diabetes in offspring have also been associated with the genital microbiome of the mother as disclosed in M. Tejesvi, et al., Sci Rep 9 (2019): 959, hereby incorporated by reference in its entirety and particularly in relation to the connection between the vaginal microbiome and Type 1 diabetes in members of a dyad.

Therefore, it is important to consider the genital microbiome and its implications on human health in the development of genital and sexual health products and therapeutics. In general, the healthy human vaginal microbiome, which is dominated by predominantly Lactobacilli species, maintains and utilizes a carbohydrate-based, antioxidant-rich environment (Chen et al. Nat Commun 8:875, 2017, hereby incorporated by reference in its entirety). These Lactobacilli produce distinct metabolites that stimulate epithelial mucus production, regulate immune cell reactivity, control pathogenic biofilm and biogenic amine formation, and prevent the colonization of the vaginal tract by pathogenic bacteria. The penile microbiome may reflect, and is correlated to, the vaginal microbiome of a female sexual partner. Dysbiosis, which refers to impaired or imbalanced microbiota, can lead to outgrowth of pathogenic, or dysbiotic, microorganisms. Dysbiotic organisms tend to prefer amino acids or proteins as energy substrates rather than carbohydrates, produce an antioxidant-poor environment (which is high in cytokine active metabolites) degradation products such as biogenic amines, and certain anti-oxidant reducing agents. These products often lead to a variety of bothersome symptoms and pathologies from odor to itching, inflammation, biofilm production (which may protect pathogens from the host immune system), mucin secretion degradation, and higher risks of viral infections and cancer. Certain life events cause an individual to be more susceptible to dysbiosis, including: ovulation, menses, birth, puberty, pregnancy, sexual initiation, non-circumcision of the male, genital mutilation of the female, diseases (e.g., diabetes), introduction of new sexual partners, post-partum, involution of the uterus, times of immunosuppression, menopause or andropause, and cancer therapy. The present disclosure is related to compositions and methods which do not elicit or minimize the occurrence of such responses and/or maintain the healthy microbiome of the anogenital and/or urogenital microbiomes.

The pathogenesis of vaginal and penile chronic dysbiosis, rely on the production of bioactive amines. A few species of dysbiotic bacteria (e.g., the bacteria present in a microbiome under dysbiosis) produce the most significant amounts of biogenic amines such as agmatine, putrescine, cadaverine, and tyramine, which increase vaginal pH and make the vaginal microbiome more inviting to other dysbiotic species. These species may also produce bacterial endotoxins such as lipopolysaccharides which have been linked to diseases, disorders, and conditions such as various cancers and dementia. Vaginal dysbiosis is often linked to concurrent male partner penile dysbiosis. This shift in metabolic states from a glycogen/carbohydrate to amino acid-rich environment results in a shift in vaginal pH away from the beneficial acidic state (e.g. pH of from 4 to 5 such as 4.5) to a more alkaline state. Such shift is initiated and perpetuated by specific bacteria including species from the genera: pathogenic Prevotella, Eggerthellia, Gardnerella, Atopobium, Megasphaera, Mobiluncus, Mageeibacillus, Gemella, Veillonella, Snethia, Mycoplasma, and some Clostridium species. It is important to note that some of these bacteria are not most abundant or solely present in a dysbiotic microbiome, confirming that the metabolic activity and the ecological role that a microbe plays in an environment is more important than its abundance. These dysbiotic bacteria produce metabolites that result in increased genital pH, offensive odors, biofilm production that interferes with host immune function, increased epithelial inflammation (which may result in symptoms such as burning, itching, and/or pain), increased mucin degradation (which may result in symptoms such as pain, roughness of the skin, and/or irritation), increased oxidative stress, cytokine and inflammasome production, and decreased growth capacity of healthy microbiome organisms. These conditions leave the individual susceptible to the development of further dysbiosis and disease states, including increased risk of poor reproductive outcomes (e.g., failed fertilization, failed embryo implantation, poor fetal growth, delivery complications), pain during sex, sexually transmitted diseases (such as high risk Human Papilloma virus (HPV) infection and persistence, genital Herpes Simplex Virus (HSV) infection, and Human Immunodeficiency Virus (HIV) infection), auto-immune conditions, cancer, and systemic disease (e.g., cognitive impairment). These conditions also have a social cost of embarrassment and social withdrawal for women with odor and discharge and for men with odor and visual roughness or inflammation of penile skin. These social aspects of genital dysbiosis are rarely discussed and under treated.

The genital microbiome of individuals may also impact sexual partners or offspring. Couples with a female partner having bacterial vaginosis (BV) show high levels of shared pathogenic bacteria with the male partner (over 17 species shared). In contrast, healthy couples also share bacterial species, but have a narrower range (e.g., less diversity) of microorganisms (e.g., commonly 4 strains of bacteria), including Lactobacillus. A menopausal woman may experience chronic bacterial dysbiosis, which favors the growth of pathogenic bacteria. This dysbiosis may be transmitted to the sexual partner of the menopausal women. For male sexual partners, penile microbiota has an important role in prostate health and disease. Penile dysbiosis of the male sexual partner may induce chronic inflammation, which may predispose the male sexual partner to subfertility, prostatitis, chronic pelvic pain syndrome, benign hyperplasia, as well as prostate cancer (Porter et al., Prostate Cancer Prostatic Dis. 21:345, 2018). The penile microbiome also affects systemic hormone levels (Id.). In another example of transmission of dysbiosis, an man with chronic penile dysbiosis can transmit dysbiosis to a female sexual partner, resulting in recurring bacterial vaginosis. Currently, women with bacterial vaginosis may undergo repeated rounds of antimicrobial therapy. However, without concurrent treatment of the male sexual partner, relapse and continued retreatment of the woman have become routine. The compositions of the present disclosure may be applied to the genital region of the male partner affording an optimization of penile microbiome. In some embodiments, application to the genitals of the male may cause the male genital microbiome to shift away from dysbiosis. In some embodiments, application to the genitals of a male in a sexual relationship may cause the genital microbiome of another member of the sexual relationship to shift away from dysbiosis. In some embodiments, another member of the sexual relationship is a female where, for example, the vaginal microbiome shifts away from dysbiosis.

Natural vaginal cleaning and protection of the woman from pathogens encountered during vaginal intercourse depends on several factors including: 1) an acidic environment (e.g., pH from 3.5 to 4.5) that facilitates constant lysis and shedding of the outer layer of vaginal epithelial cells; 2) cervico-vaginal fluids (CVF) forming in the vaginal canal that have a low salt/ion concentration compared to blood and fluids bathing inner tissues, which further facilitates lysis and shedding of the outer most vaginal epithelial cell lining, and 3) the acidic mucin-coated lining of the vaginal canal formed by mucin particles embedded in the vaginal lining, which shed to the external canal. Constant turn-over of the vaginal lining results in out-flow from the vagina, facilitating removal of pathogens before they can establish in the woman's body. However, the normally low pH and low salt environment of the vaginal canal environment can be toxic to sperm. This toxicity is mitigated during ovulation by a shift in the cervical canal derived secretions to a more neutral pH that supports sperm viability and motility (e.g., pH from 6 to 7) and by the deposition of sperm in semen during ejaculation. Semen has a more alkaline pH (e.g., pH of 7 to 8) and a higher salt concentration (3 times that of cervico-vaginal fluids (CVF) and 1.5 times that of blood). The admixing of semen and cervical fluids during vaginal intercourse at ovulation provides a neutral pH and a balanced salt environment in the vaginal vault. This environment aids in protecting sperm for their transit through the female reproductive tract (vagina and cervix). However, vaginal dysbiosis can occur if the acidic, low salt environment of the vaginal canal is not rapidly restored following the fertile phase of the woman's cycle. The compositions of the present disclosure, and particularly compositions with the particular buffers described herein, may be used before, during, or after intercourse by application to either member of the sexual dyad. Such application may facilitate a vaginal environment suitable for appropriate microbiome growth. For example, post-coital composition delivery to the vagina may restore vaginal pH and optimize vaginal health.

The impact of the shared microbiome of a sexual dyad is particularly notable during reproduction. Sexual dyads that are trying to conceive often have disrupted genital microbiota. Reproduction in humans requires an elegant interaction between the woman's host defenses in the vagina and the ejaculate of the man, which provides sperm cell transport and survival, to occur, so that fertilization of the egg follows. Subfertile women have increased levels of vaginal dysbiosis and poor vaginal microbiome resilience. Penile dysbiosis can interfere with sperm and/or semen quality. It is critical that a healthy genital microbiome be restored and maintained in couples with poor fertility. Furthermore, bacterial vaginosis and other forms of dysbiosis can increase risk of sexually transmitted diseases; cancers (e.g., cervical cancer); pelvic inflammatory disease; infertility; miscarriage; ectopic pregnancy; and poor reproductive and birth outcomes. Bacterial vaginosis is also associated with upper reproductive tract dysbiosis, which can affect the Fallopian tubes and uterus. Upper reproductive tract dysbiosis can result in infertility; miscarriage ectopic pregnancy; and pregnancy complications, including premature rupture of membranes, preterm labor, premature birth, low birth weight, chorioamnionitis, and endometritis. Pregnant women with active bacterial vaginosis have a higher risk of Cesarean—section. In some embodiments, the compositions and methods described herein may optimize the microbiome of all members of a sexual relationship with minimal effect of gametes. In some embodiments, the compositions of the present disclosure may be administered to the anogenital and/or urogenital region of a male. In some embodiments, the compositions may be administered to the anogenital and/or urogenital region of a female, and particularly, a pregnant female. Furthermore, application of the compositions of the present disclosure may have minimal (e.g., within 20% of one or more measured parameters such as motility) if not beneficial, effect on gametes (e.g., male gametes, female gametes). In some embodiments, application of the compositions of the present disclosure may have minimal (e.g., within 20% of one or more measured parameters such as motility) if not beneficial, effect on zygotes.

Additionally, urinary incontinence, diabetes, genital lichen sclerosus, interstitial cystitis, and autoimmune conditions can increase genital dysbiosis (Thomson, J. Reprod. Med. 50:513, 2005; Alam et al. Immune Netw 14:7, 2014). Individuals with such diseases can be very sensitive to disease exacerbation via ingredients or chemicals in formulations currently used for routine genital care and intimacy as disclosed in A Chung, et al., World J Urol (2019): 1-5.

Additionally, the microbiome of the fetus and newborn is shaped by the mother-fetus, mother-child dyad. The initial fetal microbiome is developed from the maternal reproductive tract microorganisms. The newborns' microbiome is seeded at birth upon exposure to maternal vaginal, fecal, and skin microbiota. Disrupting the mother-to-newborn transmission of microbiome by C-section delivery may increase the risk of celiac disease, asthma, Type 1 diabetes, and obesity in the offspring (see, Mueller et al., Trends Mol. Med. 21:109, 2015).

Genital dysbiosis is also common in individuals undergoing gender reassignment surgery. Active and ongoing management of newly created genitalia requires daily or as needed cleaning and intervention to assist in healthy genital biome development of these newly created regions (see, Weyers et al., BMC Microbiol. 9:102, 2009).

In some embodiments, subjects in need thereof may include having any one of the indications as described herein. The compositions of the present disclosure may be administered to the subject in need thereof for the treatment or prophylaxis of such conditions often related to dysbiosis. Additionally, the compositions of the present disclosure may be administered to the sexual partner of the subject in need thereof for the treatment or prophylaxis of such conditions often related to dysbiosis.

Overall, these diseases, disorders, and/or conditions are poorly controlled and poorly managed in many populations. Furthermore, the etiology of certain systemic disease conditions such as dementia (e.g., associated with incontinence and diabetes), and cancer (e.g., associated with squamous cell cancer in lichen patients) are connected with dysbiosis of the anogenital and/or urogenital regions. In some embodiments, the methods of the present disclosure may include a diagnosis from a medical professional of urinary incontinence, diabetes, genital lichen sclerosus, interstitial cystitis, dementia, cancer, and/or autoimmune conditions to the subject or a member of the sexual relationship.

Methods of Use

Disruption of the genital microbiota can lead to problems with genital discomfort, odor, burning, dyspareunia, scarring, irritation, inflammation, infection, infertility, inferior reproductive outcomes, and increase risk of autoimmune disease, sexually transmitted infections, cancer, and/or dementia. Disruption of the genital microbiota can be caused by a variety of factors including diseases (e.g., diabetes); lichen sclerosus; autoimmune diseases such as psoriasis; interstitial cystitis; hormonal changes (e.g., pregnancy, puberty, post-partum, menopause, andropause); urinary incontinence; fertility treatments; gender reassignment therapy (e.g., hormone replacement therapy and/or sex reassignment surgery); sexual initiation and introduction of new sexual partners; childbirth; immunosuppression; cancer therapy; use of alkaline soaps and washes; or use of irritating lubricants, douches, creams or medications. Topical compositions of the present disclosure are pH and isotonic specific to the urogenital and/or anogenital regions and/or genital fluids; support, promote or enhance specific genital microbiota; are non-irritating to the urogenital and/or anogenital region; do not disrupt normal anogenital mucin production; and thus, are useful for a variety of applications in the urogenital and/or anogenital region.

Without wishing to be bound by theory, the compositions of the present disclosure improve Lactobacillus dominance in the genital tissues in women of reproductive age and post-menopausal age; and girls prior to puberty. Beneficial genital microbiomes also decrease pathogens, lower vaginal pH, and/or improve Female Sexual Function Index scores with regular use. These compositions may decrease vaginal infection rates and/or urinary tract infections, especially in women in settings prone to exacerbation of these symptoms. Patients in need of these compositions may be women including women in low resource settings such as military deployment, natural disaster setting, or low-income communities, older women with dementia, pre-pubescent girls with lichen sclerosus, women with interstitial cystitis, and women with provoked vestibulodynia.

Another embodiment of the disclosure provides a relatively non-invasive intervention method that may alleviate biologic risk factors by exerting prebiotic and homeostatic properties supporting the dominance of healthy Lactobacillus species, lower pH, and reducing mucosal inflammation and trauma. In a further embodiment, reducing mucosal irritation and inflammation through lubrication with “vaginal barrier-friendly” products that minimize any detrimental effects on the vaginal mucosal barrier, and that restore physiologic pH and vaginal microbiota, the persistence and re-activation of HPV may be minimized.

In one aspect, the present disclosure provides a method of hydrating or moisturizing the urogenital and/or anogenital region of a subject comprising topically administering an effective amount of a topical, isotonic composition of the present disclosure to the urogenital and/or anogenital region of the subject. Administration of the topical, isotonic composition of the disclosure may occur as needed such as one or more times daily, twice a week, three times a week, weekly, biweekly (e.g., every two weeks), monthly, bimonthly (e.g., every two months), etc. The method may be used to hydrate or moisturize the genital tissues, including the perineum, penis, or vulva/vagina. Irritation of the skin of the penis and disruption of healthy mucin secretions that moisten and protect the penis can increase dryness, roughness and inflammation resulting in pain and discomfort. Vaginal intercourse, which introduces the penile surface to a very low pH environment can irritate the skin of the penis. Similarly, irritation of the vagina skin around the vulva can occur following ejaculation of high pH and hypertonic semen into/onto the female genital region, leading to burning and post-coital pain. Continuous washing and cleaning of the urogenital and/or anogenital region, particularly with alkaline soaps or washes, can also irritate and dry out the urogenital and/or anogenital region. By hydrating or moisturizing the urogenital and/or anogenital region and buffering pH the topical, isotonic compositions of the present disclosure may be used to promote, enhance, protect the anogenital epithelium and microbiota. In certain embodiments, topical, isotonic compositions further comprise an additional therapeutic agent, such as a topical pain-relieving agent.

Methods are provided for the prophylaxis of cancer comprising administering the compounds of the present disclosure to the urogenital and/or anogenital region of a subject in need thereof (e.g., a subject having a dysbiotic microbiome). For example, the method may cause prophylaxis of oncogenesis of cervical intraepithelial cells (e.g., in a medium, in a subject in need thereof) comprising administration of a composition of the present disclosure such as a composition comprising a metallic cofactor, a prebiotic oligosaccharide, and borneol or a prodrug thereof (e.g., bornyl acetate). In particular embodiments, the composition is administered to the anogenital and/or urogenital region (e.g., vagina). of the subject in need thereof. Because cervical intraepithelial neoplasia-associated dysbiosis is a modifiable risk cancer for cancer as disclosed in Mitra A, et al, Nat. Commun. 11 (2020): 1999, which is hereby incorporated by reference in its entirety, supporting vaginal homeostasis via administration of the compositions of the present disclosure may enhance pathogen clearance and protect against oncogenesis. Such administration may prevent or reduce vaginal microbiota shifts including dysbiosis which may lead to oncogenesis of these cancerous cells.

Administration of the compositions of the present disclosure may selectively support epithelial colonization by vaginal Lactobacillus species such as L. crispatus, L. jensenii, and L. gasseri. In some embodiments, administration may suppress pathobionts linked to cancers such as cervical cancer. In certain implementations, administration may maintain a constant or nearly constant non-inflammatory mucosal environment in comparison to baseline (no product use).

In another aspect, the present disclosure provides a method of conditioning the skin of the urogenital and/or anogenital region of a subject comprising topically administering an effective amount of a topical, isotonic composition of the present disclosure to the urogenital and/or anogenital region of the subject. Administration of the topical, isotonic composition of the disclosure may occur as needed, daily, twice a week, three times a week, weekly, biweekly (e.g., every two weeks), monthly, bimonthly (e.g., every two months), etc. The decrease in systemic estrogen levels that occurs during menopause and pregnancy can cause dry, thin, friable vulvar skin and vaginal dryness. As men age, senescence of penile epithelial tissues occurs similar to elsewhere in the body. However, high content of elastic fibers and collagen in the penis is required to allow for the repeated and regular expansion and retraction in size of the penis. The topical, isotonic compositions of the present disclosure may be used for genital skin and mucosa conditioning and genital microbiome support, particularly in an aging subject (e.g., 50 years or greater, 60 years or greater, 70 years or greater, 80 years or greater, 90 years or greater, 100 years or greater) or a subject experiencing menopause or andropause. In certain embodiments, the topical, isotonic composition further comprises an additional therapeutic agent, such as a hormone, erectile dysfunction treatment or erectile enhancement drug, premature ejaculation drug, or a combination thereof. Furthermore, diseases such as lichen sclerosus, diabetes and auto-immune diseases can make genital epithelial surfaces highly reactive to inflammation and irritation. The topical, isotonic compositions of the present disclosure may be used for genital skin and mucosa conditioning and genital microbiome support in a subject with systemic or localized epithelial diseases.

In another aspect, the present disclosure provides a method of lubricating the urogenital and/or anogenital region of a subject comprising topically administering an effective amount of a composition of the present disclosure to the urogenital and/or urogenital and/or anogenital region of the subject. Administration of the composition of the disclosure may occur prior to, during, and/or after sexual intercourse or sexual activity, as needed such as one or more times daily, twice a week, three times a week, weekly, biweekly (e.g., every two weeks), monthly, bimonthly (e.g., every two months), etc. In certain embodiments, the urogenital and/or anogenital region is the perineum, vagina, vulva, clitoris, penis, scrotum, or anus. The topical composition may be administered to the urogenital and/or anogenital region of the subject prior to, during, and/or after sexual activity. Sexual activity includes oral sex, penetrative sex (e.g., vaginal intercourse, anal intercourse), non-penetrative sex, genital contact with a body part (e.g., hand, foot), genital contact with an object (e.g., sex toy), masturbation, dry humping (genital rubbing), or any combination thereof. In certain embodiments, the composition is administered to the urogenital and/or anogenital region of the subject for use with a sex toy. In additional embodiments, the composition is administered to a medical device, contraceptive device, or sex toy in alternative or in addition to administration to the urogenital and/or anogenital region. For example, the composition may be administered to the interior or exterior of a condom, to the interior or exterior of a sex toy, to the exterior or interior of a tampon, to the exterior of a menstrual cup, to the exterior of a diaphragm, or to the exterior of a vaginal ultrasound or speculum prior to contact with the subject's urogenital and/or anogenital region.

During vaginal, heterosexual intercourse, the penis is introduced to low pH vaginal secretions. Sexual intercourse frequently leads to dysbiosis and production of odorous amines from anaerobic bacteria. Most commercial lubricants contain irritating and damaging ingredients, such as photo-oxidized oils, silicones, and chemicals, and are hypertonic (e.g., five times the level of physiologic fluids), which can further irritate the urogenital and/or anogenital region when used during sexual activity, such as intercourse or masturbation. The topical, isotonic compositions of the present disclosure may provide urogenital and/or anogenital lubrication and genital microbiome support without irritating the urogenital and/or anogenital region in a subject. In certain embodiments, the composition further comprises an additional therapeutic agent, such as a hormone, a drug for treating vaginal atrophy (e.g., genitourinary syndrome of menopause), a drug for enhancing sexual responsiveness (e.g. orgasmic latency), erectile dysfunction treatment or erectile enhancement drug, a drug for premature ejaculation, an agent that enhances vasodilation, a microbicide to prevent STDs, a chemical contraceptive, or a combination thereof.

In another aspect, the present disclosure provides a method of decreasing irritation or inflammation of the urogenital and/or anogenital region of a subject comprising topically administering an effective amount of a topical, isotonic composition of the present disclosure to the urogenital and/or anogenital region of the subject. Administration of the topical, isotonic composition of the disclosure may occur as needed, daily, twice a week, three times a week, weekly, biweekly (e.g., every two weeks), monthly, bimonthly (e.g., every two months), etc.

In another aspect, the present disclosure provides a method of preventing cervical cancer, wherein the compositions of the present disclosure are administered to a subject in need thereof. Administrations of these compositions may serve as a prebiotic for beneficial genital microbiota growth, including enhanced Lactobacillus dominance, and enhancing high risk HPV clearance. The prophylactic use of compositions of the disclosure may allow for Lactobacillus dominance which may serve to prevent or reduce the occurrence of cervical cancer and STDs, including but not limited to HIV, HSV, HPV, and high-risk HPV. Without being bound by theory, Lactobacillus dominance may prevent or reduce the occurrence of cervical cancer or STDs by stopping pathogen adherence to vaginal cells and by producing chiral-lactic acid that protects the cervix. In certain embodiments, the compositions of the disclosure may be topically administered to the urogenital and/or anogenital region, including the vagina, through an applicator described herein. Administration of the topical, isotonic composition of the disclosure may occur as needed, daily, twice a week, three times a week, weekly, biweekly (e.g., every two weeks), monthly, bimonthly (e.g., every two months), etc. For example, a method of preventing or reducing the occurrence or persistence of cervical cancer or an STD, including but not limited to, HIV, HSV, HPV, or high-risk HPV, in a subject, comprises topically administering an effective amount of a topical, isotonic composition of the disclosure to the urogenital and/or anogenital region of the subject in need thereof. This method may also comprise administration of a point-of-care pH sensor, which may communicate with an application accessible through a computer or a mobile device, thereby creating an integrated delivery and pH tracking system for the treatment or prophylaxis of cervical cancer or STDs. In those methods for the treatment or prophylactic use of the compositions of the disclosure to prevent or reduce the occurrence or persistence of cervical cancer or STDs in a subject in need thereof, the pH sensor may be a vaginal pH sensor for measuring and monitoring the pH in the vagina.

A further embodiment may provide for a method of correcting the pH or microbiota of a subject (e.g., the penile microbiota, the vaginal microbiota) or a sexual partner of the subject comprising topically administering to the subject in need thereof, an effective amount of a topical, isotonic composition described herein, to the urogenital and/or anogenital region of the subject in need thereof. Another embodiment may be directed to a method of optimizing Lactobacillus dominance of the microbiome environment of the urogenital and/or anogenital region (e.g., vagina, penis) of the subject and/or a sexual partner of the subject comprising topically administering an effective amount of a composition described herein, to the urogenital and/or anogenital region of the subject in need thereof. Administration of the topical, isotonic composition of the disclosure may occur as needed, daily, twice a week, three times a week, weekly, biweekly (e.g., every two weeks), monthly, bimonthly (e.g., every two months), etc.

In another aspect, the present disclosure provides a method of decreasing bothersome vaginal symptoms in older women with postmenopausal Genitourinary Syndrome of Menopause, BV, urinary incontinence, pelvic floor prolapse or other forms of pelvic floor disease wherein the compositions of the present disclosure are administered to a subject or sexual partner of a subject in need thereof, including optional concurrent use with a vaginal pessary for prolapse management.

In another aspect, the present disclosure provides a method of cleaning the urogenital and/or anogenital region of a subject comprising topically administering an effective amount of a topical, isotonic composition of the present disclosure to the urogenital and/or anogenital region of the subject. The use of harsh alkaline soaps and washes (pH ˜8-11) to clean the urogenital and/or anogenital region (e.g., penis or vulva/vagina) can disrupt the microbiota, leading to unpleasant genital odor, irritation, and BV. Semen, sweat, and some lubricants also have an alkaline pH. Many commercial diaper wipes have very acidic pH (e.g., about pH 2.8) or alkaline pH (e.g., about pH 8), which can depress or elevate, respectively, the normal skin pH of the urogenital and/or anogenital area following use (Priestly et al., Pediatr. Dermatol. 13:14, 1996). The normal penile skin has a pH of about pH 4.5-pH 6. The normal pH of the vulva/vagina is generally acidic due to the Lactobacilli in the microbiota, although vulvar pH is higher than that of vagina. However, the pH of the female genital region varies depending on the life stage and menstrual cycle stage. During most of the menstrual cycle, the female genital region is often at a pH of about 4.5. However, during ovulation, the cervical secretions becomes more neutral at about pH 6 to about pH 7 (which facilitates rapid sperm transport through the cervix). During pregnancy and after menopause, the pH of these secretions is often at about 5. These states are prone to vaginal dysbiosis. Postmenopausal women have high rates of BV (approaching 50%) and BV rates increase over time after menopause. Exposure to alkaline pH associated with BV or dysbiosis can disrupt the genital microbiota, leading to increased pathogenic bacteria growth and production of offensive biogenic amines, increased vaginal symptoms, sexually transmitted diseases, and cancers. Individuals who have increased sweating in the groin area due to inherent physiology, weight gain or frequent exercise, in combination with an imbalanced penile or vaginal microbiome, can also have increased offensive genital odor that can impact quality of life. Moreover, genital odor complaints and dysbiosis are more significant in uncircumcised men due to the collection of secretions in the preputial fornix. The topical compositions of the present disclosure are non-irritating, and pH and isotonic specific to the urogenital and/or anogenital region of interest.

In another aspect, the present disclosure provides a method of decreasing bothersome penile skin conditions, resulting in redness, roughness, scabbing, scaling, itching, and odor wherein the compositions of the present disclosure are administered to a subject in need thereof.

In another aspect, the present disclosure provides a method of decreasing Type 1 diabetes in offspring by enhanced Lactobacillus dominance in the mother's vagina prior to birth wherein the compositions of the present disclosure are administered to the mother in need thereof or a sexual partner of the mother. The connection between diseases such as Type 1 diabetes in offspring have been associated with the genital microbiome of the mother as disclosed in M. Tejesvi, et al., Sci Rep 9 (2019): 959, hereby incorporated by reference in its entirety and particularly in relation to the connection between the vaginal microbiome and Type 1 diabetes in members of a dyad.

In yet another aspect, the present disclosure provides a method of supporting, enhancing, or promoting the genital microbiota of a subject or a sexual partner of a subject comprising topically administering an effective amount of a composition of the present disclosure to the genital region of the subject. In certain embodiments, the topical, isotonic composition is prebiotic for Lactobacillus species growth. In certain embodiments, the topical, isotonic composition may further comprise at least one probiotic bacterial species (e.g., Lactobacillus species).

In yet another aspect, the present disclosure provides a system and method of hydrating, conditioning, cleansing, or lubricating the urogenital and/or anogenital region of one member or both members of a sexual dyad throughout a reproductive cycle, in order to enhance reproductive outcomes. In certain embodiments, the sexual dyad comprises at least one female. In certain embodiments, the sexual dyad is a heterosexual dyad. The ovarian cycle may include the follicular phase (pre-ovulatory phase), ovulation, and luteal phase (post-ovulatory phase). The uterine cycle includes menses, proliferative phase, and secretory phase. Embodiments of this system provide urogenital and/or anogenital cleansing and lubrication at a pH consistent with the healthy function of the penile, clitoral, vaginal, and/or vulvar ecosystem(s), the urogenital ecosystems, cervical fluids, sperm, and semen. The system may provide urogenital and/or anogenital cleansing and lubrication at a pH and osmolality consistent with the healthy function of cervical fluids, and sperm in the vagina post-ejaculation. Specific embodiments of compositions of the present disclosure are used by one or both members of the sexual dyad during the non-fertile portion of the cycle (e.g., the follicular phase) (Step 1). In certain embodiments, the follicular phase refers to the period of time from day 0 of menstrual cycle up to ovulation. These compositions are used for cleansing, and as a leave-in conditioner to optimize the healthy genital microbiome, and as a coital lubricant applied to the vaginal canal prior to intercourse or sexual activity. The compositions may be buffered, muco-adhesive and have a tonicity of 150 mOsmo/kg and a pH of 4.5. Other embodiments of compositions of the present disclosure are then used during the fertile phase (Step 2). As used herein the fertile window or fertile phase includes the luteinizing hormone surge that occurs 24 to 36 hours before ovulation and ovulation. In certain embodiments, the fertile phase refers to the period of 6 days, 5 days, 4 days, 3 days, 2 days, or 1 day before ovulation through 24 hours or 36 hours after ovulation.

These compositions may be used for cleansing, and as a leave-in conditioner to enhance the healthy genital microbiome, and as a coital lubricant applied to the vaginal canal prior to intercourse or sexual activity. These compositions have a tonicity of 240 mOsmo/kg and a pH of from pH 3.5 to pH 6.8 (e.g., 4-6). In certain embodiments, sperm function is protected at the lower pH of the vagina by compositions with enhanced anti-oxidant activity, and improved cervical mucus quality to support rapid transport of sperm out of the acidic vaginal environment. Protecting post-ejaculatory sperm during transport through a composition which is at a lower pH (e.g. pH from 3.8 to 6.8, pH from 4 to 5) than that of typical cervical mucus (e.g. pH from 6.8 to 7.4), may limit subsequent dysbiosis and BV risk in the mother. Furthermore, the acidic compositions of the present disclosure may have minimal effect (e.g., more than 20% of one or more measured parameters such as sperm motility) on male gametes, female gametes, and/or zygotes. In certain embodiments, from 36 hours following ovulation, the compositions from Step 2 are discontinued (e.g., luteal phase) and compositions from Step 1 are used to facilitate return of the vaginal canal to an acidic, hypotonic environment. In certain embodiments, the period of time outside the fertile window, including before, after, or both before and after the fertile window is referred to as the non-fertile phase or time. In certain embodiments, the compositions of the present disclosure may adjust the postcoital admix of fluids (e.g., CVF, semen) in the vagina to a pH of from 6 to 8 and tonicity of 240 mOsmo/kg. In certain embodiments, the compositions of the present disclosure adjust the postcoital admix of fluids (e.g., CVF, semen) in the vagina to a pH of from 4 to 8 or from 4 to 6 or from 5 to 7.5. These adjustments may adjust the tonicity of the environment to a tonicity of from 240 to 300 mOsmo/kg. Typically, sperm function is preserved during exposure to the composition for the required time for sperm transport through the human vagina (e.g., for from 20 minutes to 40 minutes, for 30 minutes). Such mechanisms are discussed in F. Nakano, Medical Express 2 (2015): ISSN 2318-8111, hereby incorporated by reference in its entirety.

In certain embodiments, the pH of the composition is formulated at a pH to match to the normal, physiological genital fluid pH (e.g., CVF, urethral secretions, semen) or at a pH appropriate for the particular method of use. In certain embodiments, the pH of the topical, isotonic composition ranges from 3 to 8 or from 3.5 to 7.5 or from 3.5 to 6.8. In a particular embodiment, the pH of the topical, isotonic composition is from pH of 3.5-5 or from 4-4.5, or from 5-5.5, or from 5-6.8. Topical, isotonic compositions having a pH ranging from pH 3.5 to pH 6.8, are particularly suited for administration to a subject that is aged from puberty to menopause/andropause, e.g., 18 years of age to 50 years of age, 18 years of age to 55 years of age, 18 years of age to 60 years of age, or a child at least one year old. In certain embodiments, the pH of the composition ranges from pH 5 to pH 8. In some embodiments, compositions having a pH ranging from pH 4 to pH 7 may be particularly suited for administration to an infant aged from 0 to 12 months old, a senior adult of at least 60 years of age, an adult (male or female) of reproductive age (e.g., ranging from 18 years to 50 years) who is actively trying to conceive, or both members of a heterosexual dyad which is actively trying to conceive.

The methods provided in the present disclosure may be used on an animal subject (e.g., mammalian, bovine, canine, feline, equine, porcine, ovine, avian, rodent, lagomorph, caprine, non-human primate), preferably a human subject. In certain embodiments, the subject is a male, a female, an intersex subject, a non-binary gendered subject, or a subject of any other gender designation. In certain embodiments, the subject is an infant, a child, or an adult. In certain embodiments, the subject is an adult male of reproductive age (e.g., ranging 18 years to 50 years) that is trying to conceive or adult female of reproductive age (e.g., ranging from 18 years to 50 years) that is trying to conceive. In certain embodiments, the subject is a female in menopause or male in andropause. In certain embodiments, the subject is an infant (aged from 0 to 12 months old), a child at least 1 year old; an adult ranging from 18 years to 50 years of age, 18 to 55 years of age, or 18 to 60 years of age; or a senior adult of at least 50 years, at least 55 years, at least 60 years, or at least 65 years of age. In certain embodiments, a senior is at least 60 years old. In certain embodiments, a senior is at 70 years old. In certain embodiments, a senior is at least 80 years old.

In any of the embodiments provided in the present disclosure, the composition may be administered to an individual subject as part of a treatment regimen, to members of a non-monadic sexual relationship (e.g., sexual dyad, sexual triad) as a part of a treatment regimen, or to both members of a non-sexual dyad. In certain embodiments, the sexual dyad is a homosexual dyad, a heterosexual dyad, or other sexual orientation dyad. In certain embodiments, a non-sexual dyad is parent-child dyad (e.g., mother-child or father-child), caregiver-child dyad, caregiver-adult patient dyad, or caregiver-senior patient dyad.

In some embodiments, the method of optimizing the beneficial microbiome growth in the genital region of a subject in need thereof may comprise application of a pharmaceutical composition having an acidic pH to the genital region of the subject or to the genital region of a sexual partner of the subject in need thereof. In some embodiments, optimizing the beneficial microbiome growth comprises inhibiting pathogenic bacterial growth. In various implementations, the optimizing the beneficial microbiome growth comprises promoting beneficial bacterial growth. Additionally, application of the pharmaceutical composition may have minimal effect to the gametes (e.g., male gametes, female gametes) of the subject or the sexual partner of the subject and/or has minimal effect on zygote formed during intercourse between the subject and a sexual partner. In various implementations, the application may have minimal effect to the male gametes in any genital fluids secreted from the subject or the sexual partner of the subject. For example, the male gametes may have minimal change (e.g. less than 20%) in their motility and/or concentration and/or vitality and/or morphology and/or oxidation-reduction potential and/or sperm DNA fragmentation and/or sperm mitochondrial membrane potential and/or survival and/or sub-cellular alterations (e.g., as compared to gametes produced without application of the composition). These parameters may be measured by any of the methods described herein, and in particular those described in the Examples.

Compositions

The present disclosure provides genital products that can optimize genital health and modulate the metabolism of the genital microbiome. In some embodiments, the compositions described herein optimize the penile microbiome. In some embodiments, the compositions described herein optimize the vaginal microbiome of a sexual partner of the user applying the compositions described herein. These topical, isotonic compositions of the present disclosure may include components for supporting growth, dominance, resilience, and metabolism of beneficial lactic-acid producing bacteria and components for inhibiting dysbiotic bacterial growth and metabolism. By influencing the metabolism of the genital microbiome, the growth and metabolic activity of beneficial bacterial species (e.g., Lactobacillus, Streptococcus, Staphylococcus, Corynebacteria) can be enhanced, and the growth and metabolism of bacterial species that lead to genital dysbiosis and disease can be inhibited to restore and maintain genital health. By treating male members of dyads, triads, and higher order social groups of significance, the health of all the members of dyads, triads, or larger social groups can also be improved. Furthermore, people in high stress, crowded, or unhygienic situations, such members of the military, incarcerated individuals, people living in dormitories or shelters, people in man-made or natural disasters can also form a dyad or other social grouping with extensive sharing of anogenital microbiota through daily interactions. People living in such settings can benefit from the improved cleaning and personal care offered by the compositions of the present disclosure.

The pharmaceutical compositions of the present disclosure are maintained at an acidic pH. Without wishing to be bound by theory, an acidic pH is able to promote and optimize microbiome health and, in some embodiments, may have minimal or beneficial effect on gametes of members of a sexual relationship. In some embodiments, the composition may be a topical, isotonic compositions comprising a prebiotic oligosaccharide, a metal co-factor, and bornyl acetate (e.g., the composition comprises an essential oil comprising bornyl acetate), at a pH ranging from about 3.5 to about 8. In certain embodiments, the composition may further comprise a pH modifying agent to adjust the final pH of the composition to the target or desired pH. The pH modifying agent may comprise an acidifying agent, an alkalinizing agent, and/or both an acidifying agent and an alkalinizing agent. In certain embodiments, the pH modifying agent is in an amount ranging from 0.01% to 10% (e.g., from 0.01% to 1% or from 0.01% to 0.1% or from 0.1% to 1% or from 1% to 10%) by weight of the composition. Exemplary acidifying agents include, but are not limited to, organic acids such as α-hydroxy acids, citric acid, lactic acid, formic acid, glycolic acid, acetic acid, propionic acid, butyric acid, caproic acid, oxalic acid, maleic acid, benzoic acid, carbonic acid, and the like. Exemplary alkalinizing agents include ammonia, ammonium carbonate, diethanolamine, monoethanolamine, potassium hydroxide, potassium phosphate dibasic, sodium bicarbonate, sodium borate, sodium carbonate, sodium hydroxide, sodium lactate, sodium phosphate dibasic, trolamine, or any combination thereof.

In certain embodiments, the pH of the composition is formulated at a pH to match to a normal, physiological fluid pH (e.g., cervo-vaginal secretions, semen) or the epithelial surface of the genital tissue, or the anogenital epithelium (e.g., mucosa, skin). In certain embodiments, the pH of the composition is less than (e.g., from 3 to) 6.8 or less than 6.5 or less than 6.2 or less than 6.0 or less than 5.7 or less than 5.5, or less than 4.5. In certain embodiments, the pH of the topical isotonic composition is 3.5 to 7.5, 3.5 to 6.8, 3.5 to 6.0, 3.5 to 5.7, 4 to 5, or 4.25 to 4.75. In a particular embodiment, the pH of the topical, isotonic composition is 3.5, 3.8, 4, 4.1, 4.2, 4.25, 4.3, 4.4, 4.5, 4.6, 4.7, 4.75, 4.8, 4.9, 5, 6.5, or 6.8.

In certain embodiments, topical, isotonic compositions of the present disclosure further comprise a buffering agent. The buffering agent may be in an amount ranging from 0.01% to 0.9% by weight of the composition. A buffering agent refers to a compound or a mix of compounds that, when present in a solution, resists changes in the pH of the solution when small quantities of acid or base are added or upon dilution with a solvent or bodily fluid. Buffer capacity is a measure of the resistance to change in the pH of a solution when acids or bases are added to the solution. The total amount of the buffering agent (e.g., conjugate acid-base pair) is selected such that the pH of the composition is maintained at the desired pH or range of pH values. Thus, the greater the amount of the buffering capacity, the more resistant the pH of the composition is to change. In certain embodiments, a buffering agent contains an acidic species to neutralize hydroxide (OH⁻) ions and a basic species to neutralize hydrogen (H⁺) ions. However, the acidic and basic species of the buffering agent should not consume each other through a neutralization reaction.

In certain embodiments, the buffering agent is a simple buffered solution comprising a weak acid and a salt of the weak acid or a weak base and a salt of the weak base. Thus, the buffering agent can include a weak acid-base conjugate pair or weak base-acid conjugate pair. Examples of weak acid/salt of weak acid and weak base/salt of weak base parings include citric acid/sodium citrate, lactic acid/sodium lactate, acetic acid/sodium acetate, monosodium phosphate/disodium phosphate, propionic acid/sodium propionate, butyric acid/sodium butyrate, carbonic acid/sodium bicarbonate, malic acid/sodium malate, ascorbic acid/sodium ascorbate benzoic acid/sodium benzoate, succinic acid/sodium succinate and sodium borate/boric acid. In certain embodiments, the buffering agent comprises unrelated weak acid-base pairs. Examples of such combinations include disodium phosphate/citric acid, disodium phosphate/lactic acid, monosodium phosphate/sodium lactate, monosodium phosphate/sodium citrate, sodium citrate/lactic acid, sodium lactate/citric acid, monopotassium phosphate/citric acid, monopotassium phosphate/lactic acid, monopotassium phosphate/sodium lactate, monopotassium phosphate/sodium citrate, monopotassium citrate/lactic acid, and potassium lactate/citric acid. In addition, for multivalent anions, the calcium salt rather than sodium salt may be used (e.g., calcium citrate). Example buffer can also include gluconolactone/gluconic acid.

In certain embodiments, the buffering agent is selected such that the buffering agent's acid form has a pKa the same as or close to the desired pH of the composition or a pH within the desired range of pH values, functions with a similar buffering capacity to surfaces and fluids that physiologically occur in genital region (e.g., vaginal mucin-acidic barrier, cervico-vaginal secretions, semen, menses-flow, or a combination thereof), or maintains pH to that of the target epithelial surface (Rastogi et al., Contraception. 93:337, 2016). In certain embodiments, a buffering agent comprises a monocarboxylate, a dicarboxylate, a carboxylic acid, or a combination thereof. In some embodiments, a buffering agent may comprise an acetate, borate, citrate, fumarate, lactate, malate, malonate, nitrate, phosphate, propanoate, succinate, tartrate, tromethamine, or any combination thereof. In some embodiments, a buffering agent comprises lactic acid, sodium lactate, sodium phosphate (monobasic, dibasic, or both), potassium phosphate (monobasic, dibasic, or both), sodium citrate, potassium citrate, calcium citrate, acetic acid, sodium acetate, citric acid, disodium citrate, trisodium citrate, boric acid/sodium, succinic acid, sodium succinate, gluconolactone, disodium succinate, tartaric acid, sodium tartrate, sodium ascorbate, ascorbic acid, tromethamine (Tris), or any combination thereof. In certain embodiments, the buffering agent in the compositions may comprise citric acid, a sodium phosphate such as monosodium phosphate and/or disodium phosphate, lactic acid and sodium lactate, gluconolactone, or mono- or disodium phosphate and lactic acid.

In certain embodiments, the compositions of the present disclosure may further comprise a preservative. The preservative may be in an amount of 0.001% to 4% (e.g., 0.001% to 1%) by weight of the composition. Exemplary preservatives include, but are not limited to, caprylyl glycol, cranberry extract, dichlorobenzyl alcohol, gluconolactone, green tea extract, oleuropein, pentylene glycol, phenethyl alcohol, pomegranate extract, potassium benzoate, propanediol, resveratrol, hydantoin, benzoic acid, benzyl alcohol, dehydroacetic acid, ethylhexyl glycerin, Lactobacillus ferment, pentylene glycol, potassium sorbate, sodium benzoate, sodium dehydroacetate, glyceryl caprylate, sodium salicylate, Euxyl® K 903 (Benzoic Acid 11-13%; Dehydroacetic acid 6.5-7.5%; Benzyl alcohol 78-84%, Lincoserve™ BDHA (Dehydroacetic acid 7.5-8.5%; Benzyl alcohol 86-88%, Lincoserve™ WpH-LO (Pentylene Glycol 30-40%; Propanediol 25-35%; Ethylhexyl Glycerin 1-10%; Caprylyl Glycol 10-20%, Linatural™ Ultra-3 (Pentylene Glycol 30-45%; Propanediol 45-55%; Phenethyl Alcohol 5-15%, Linatural™ MBS-1 (Ethylhexyl Glycerin 1-10%; Propanediol 75-85%; Potassium Sorbate 1-10%, Lincoserve™ SSB (water 55-65%; Sodium Benzoate 20-30%; Sodium Salicylate 10-20, or any combination thereof. In various implementations, the preservative may be one or more α-hydroxy acids such as lactic acid, citric acid, glycolic acid, or combinations thereof.

In certain embodiments, the topical, isotonic compositions of the present disclosure are sterile and/or preservative-free (e.g., less than 0.1%, or less than 0.01%, or less than 0.001% by weight of the composition). Although lower pH may work synergistically to improve the efficacy of preservatives (e.g., in terms of minimizing or inhibiting microbial growth), acidic compositions having preservatives may not have a similar effect on gametes (see Examples). Some acidic compositions (e.g., pH 4-5) having preservatives have been shown to have less effect on gametes such as sperm than those at higher pH (e.g., pH 6.5-7.5). In some embodiments, the preservative may be added in an amount to provide anti-microbial efficacy of pathogenic growth including bacterial or fungal growth. In some embodiments, the preservative decreases the growth of pathogenic organisms such as E. coli, P. aeruginosa, S. aureus, A. brasiliensis, C. albicans or combinations thereof in the composition.

Pharmaceutical compositions such as topical, isotonic compositions of the present disclosure may comprise a prebiotic oligosaccharide in an amount ranging from 0.001% to 5% by weight of the composition. Prebiotic oligosaccharide refers to oligosaccharides substrates that induce the growth or activity of beneficial microorganisms of the microbiota, e.g., genital microbiota. In certain embodiments, a prebiotic is non-digestible and resistant to breakdown by stomach acid and enzymes in the human gastrointestinal tract, selectively fermented by genital microbiota (e.g., beneficial genital microbiota), selectively target and stimulate the growth and activity of specific genital microbiota (e.g., healthy and/or beneficial genital microbiota), or any combination thereof.

One or more prebiotics may be added to composition. Suitable prebiotics may include oligosaccharides, particularly galactooligosaccharide (GOS), palatinoseoligosaccharide, soybean oligosaccharide, gentiooligosaccharide, xylooligomers, non-degradable starch, lactosaccharose, lactulose, lactitol, maltitol, polydextrose, or the like. Examples of prebiotic oligosaccharides that may be used in the compositions of the present disclosure include raffinose, lactulose, trehalose, galactooligosaccharide, alpha-glucan oligosaccharide, beta-glucan oligosaccharide, maltose, xylose, fructooligosaccharide, isomaltooligosaccharide, inulin, pectin, or any combination thereof. In certain embodiments, a prebiotic oligosaccharide does not include xylose.

The compositions of the present disclosure may also comprise a metal co-factor. Metal co-factors may be metallic ions, or salts thereof, which often act as a catalyst for an enzyme's activity. Specifically, the metal co-factors may assist enzymes involved in glycosylation of proteoglycans, such as glycosaminoglycans, which are involved in a variety of physiological functions including barrier immune protection, epithelial hydration and providing viscosity to natural bodily fluids, such as cervico-vaginal secretions, penile foreskin secretions, smegma, distal urethral sections, and semen. The metal co-factor may be requisite in some conditions for Lactobacillus growth. Pathogenic bacteria may sequester such metal co-factors, as an act of establishing dominance over beneficial genital microbiota. The metal co-factor may be present in an amount ranging from 0.0001% to 0.1% by weight of the composition. A metal co-factor may comprise zinc, selenium, molybdenum, manganese, cobalt, iron, copper, including salts thereof, or any combination thereof. The metal co-factor may be added to the composition as a salt of the co-factor metallic ion comprising a counterion, for example, the chloride salt. For example, the metal co-factor may comprise Mn²⁺, Mn³⁺, Mn⁴⁺, Mn⁶⁺, Mn⁷⁺, Zn¹⁺, Zn²⁺, Se²⁺, Se⁴⁺, Se⁶⁺, Co²⁺, Co³⁺, Fe²⁺, Fe³⁺, Cu¹⁺, Cu²⁺, or pharmaceutically acceptable salts (e.g. the chloride salt) thereof, or hydrates of any of the foregoing. In some embodiments, the metal co-factor is manganese (II) chloride. It will be understood that some hydrates of these metallic salts may dissociate to form the metal-cofactor in the topical isotonic solutions.

The compositions may include borneol, pharmaceutically acceptable salts, esters, and prodrugs thereof. For example, the compositions may comprise bornyl acetate. Bornyl acetate is a compound commonly found in pine needles, valerian root, fir needles, hemlock, cypress, rosemary, and occasionally juniper berries and spearmint. Bornyl acetate has acetylcholinesterase enzyme inhibitory, anti-inflammatory, and analgesic activity as shown for several indications in described in L Yang, et al., Biomed Pharmacother 103 (2018): 234-239, S. Lu et al. Biomed Res Int 2018 (2018): 3589874, T Zhang et al. Front Pharmacol 8 (2017): 786, and D Szwajgier et al., Curr Alzheimer Res 16 (2019): 963, each hereby incorporated by reference in their entirety. In some embodiments, the pharmaceutical composition comprises bornyl acetate or pharmaceutically acceptable salts or prodrugs thereof. In some embodiments, the pharmaceutical composition comprises borneol or pharmaceutically acceptable salts thereof or prodrugs (e.g., ester prodrugs) of any of the foregoing.

In some embodiments, the compositions of the present disclosure may comprise more than 0.0001% bornyl acetate by weight of the composition (e.g., more than 0.00025% bornyl acetate, from 0.0001% to 1% bornyl acetate, from 0.0001% to 0.5% bornyl acetate, from 0.0001% to 0.3% bornyl acetate, from 0.0001% to 0.2% bornyl acetate, from 0.0001% to 0.1% bornyl acetate, from 0.0001% to 0.001% bornyl acetate, from 0.001% to 0.01% bornyl acetate, from 0.01% to 0.1% bornyl acetate, from 0.1% to 1% bornyl acetate, from 0.01% to 0.3% bornyl acetate, from 0.1% to 0.3% bornyl acetate, from 0.00025% to 0.20% bornyl acetate, 0.0005% to 0.20% bornyl acetate, from 0.0001% to 0.15% bornyl acetate, from 0.00025% to 0.15% bornyl acetate, from 0.001% to 0.06% bornyl acetate) by weight of the composition. In some embodiments, the composition may comprise less than 0.3% (e.g., less than 0.15%, less than 0.015%, from 0.0001% to 0.015%, from 0.00025% to 0.015%) bornyl acetate by weight of the composition. In some embodiments, the compositions of the present disclosure may comprise more than 0.0001% borneol, pharmaceutically acceptable salts thereof, esters (e.g., C₁-C₇ esters), or prodrugs of any of the foregoing (e.g., bornyl acetate) by weight of the composition (e.g., more than 0.00025% borneol, pharmaceutically acceptable salts thereof, or prodrugs of any of the foregoing, from 0.0001% to 1% borneol, pharmaceutically acceptable salts thereof, or prodrugs of any of the foregoing, from 0.0001% to 0.5% borneol, pharmaceutically acceptable salts thereof, or prodrugs of any of the foregoing, from 0.0001% to 0.3% borneol, pharmaceutically acceptable salts thereof, or prodrugs of any of the foregoing, from 0.0001% to 0.2% borneol, pharmaceutically acceptable salts thereof, or prodrugs of any of the foregoing, from 0.0001% to 0.1% borneol, pharmaceutically acceptable salts thereof, or prodrugs of any of the foregoing, from 0.0001% to 0.001% borneol, pharmaceutically acceptable salts thereof, or prodrugs of any of the foregoing, from 0.001% to 0.01% borneol, pharmaceutically acceptable salts thereof, or prodrugs of any of the foregoing, from 0.01% to 0.1% borneol, pharmaceutically acceptable salts thereof, or prodrugs of any of the foregoing, from 0.1% to 1% borneol, pharmaceutically acceptable salts thereof, or prodrugs of any of the foregoing, from 0.01% to 0.3% borneol, pharmaceutically acceptable salts thereof, or prodrugs of any of the foregoing, from 0.1% to 0.3% borneol, pharmaceutically acceptable salts thereof, or prodrugs of any of the foregoing, from 0.00025% to 0.20% borneol, pharmaceutically acceptable salts thereof, or prodrugs of any of the foregoing, 0.0005% to 0.20% borneol, pharmaceutically acceptable salts thereof, or prodrugs of any of the foregoing, from 0.0001% to 0.15% borneol, pharmaceutically acceptable salts thereof, or prodrugs of any of the foregoing, from 0.00025% to 0.15% borneol, pharmaceutically acceptable salts thereof, or prodrugs of any of the foregoing, from 0.001% to 0.06% borneol, pharmaceutically acceptable salts thereof, or prodrugs of any of the foregoing) by weight of the composition. In some embodiments, the composition may comprise less than 0.3% borneol, pharmaceutically acceptable salts thereof, or prodrugs of any of the foregoing (e.g., less than 0.15% borneol, pharmaceutically acceptable salts thereof, or prodrugs of any of the foregoing, less than 0.015% borneol, pharmaceutically acceptable salts thereof, or prodrugs of any of the foregoing, from 0.0001% borneol, pharmaceutically acceptable salts thereof, or prodrugs of any of the foregoing to 0.015% borneol, pharmaceutically acceptable salts thereof, or prodrugs of any of the foregoing, from 0.00025% borneol, pharmaceutically acceptable salts thereof, or prodrugs of any of the foregoing to 0.015% borneol, pharmaceutically acceptable salts thereof, or prodrugs of any of the foregoing), by weight of the composition.

Borneol, bornyl acetate, pharmaceutically acceptable salts thereof, or prodrugs of any of the foregoing typically have at least one chiral center. The compositions may comprise the enantiomerically pure compound, enantiomeric mixtures of an indicated compound, or racemic mixtures of the enantiomer. For example, bornyl acetate may be present as (+)-bornyl acetate, (−)-bornyl acetate, racemic mixtures thereof, or enantiomeric mixtures thereof (e.g., a weight ratio of (+)-bornyl acetate to (−)-bornyl acetate of from 100:1 to 1:100 or 50:1 to 1:50 or 25:1 to 1:25 or 10:1 to 1:10).

In some embodiments, the compositions of the present disclosure may also comprise an essential oil which comprises bornyl acetate, a monoterpene ester, in an amount ranging from 0.005% to 0.5% by weight of the composition. In certain embodiments, the essential oil comprising bornyl acetate may be an essential oil selected from Juniperus communis, Juniperus occidentalis, Juniperus scopulorum, Abies sibirica, Abies alba, Abies balsamea, Abies fraseri, Abies grandis, Abies spectabilis, Abies koreana, Abies procera, Abies nordmanniana, Abies magnifica, Abies pinsapo, Abies lasiocarpa, Abies concolor, Pseudotsuga menziesii, Ambrosia trifida, Pinus mugo, Romanian solidago, Ribes nigrum, Laurus nobilis, Rosmarinus officinalis, or any combination thereof. In some embodiments, the essential oil comprising bornyl acetate comprises bornyl acetate in an amount of at least 5% by weight of the essential oil. In some embodiments, the essential oil comprising bornyl acetate comprises bornyl acetate in an amount ranging from 5% to 30% or from 10% to 30% by weight of the essential oil. Accordingly, in some embodiments, the topical, isotonic compositions may comprise more than 0.00025% bornyl acetate (e.g., from 0.00025% to 0.15% bornyl acetate, 0.0005% to 0.15% bornyl acetate) by weight of the composition. In some embodiments, the topical isotonic compositions may comprise from 0.0001% bornyl acetate to 0.3% bornyl acetate by weight of the composition. In certain implementations, the topical isotonic compositions may comprise an essential oil comprising bornyl acetate such that from 0.0001% bornyl acetate to 0.3% bornyl acetate (derived from the essential oil) by weight of the composition.

In certain embodiments, compositions of the present disclosure further comprise a bisabolene. The bisabolene may be in an amount ranging from 0.0001% to 0.1%. Bisabolene has anti-inflammatory, wound healing, skin strengthening, anti-tumor and/or analgesic activity. In certain embodiments, bisabolene is obtained from Commiphora guidotti, Pallines spinosa, Platanus chiapensis, Platanus gentryi, Platanus kerrii, Platanus mexicana, Platanus oaxacana, Platanus occidentalis, Platanus orientalis, Platanus racemosa, Platanus rzedowskii, Platanus wrightii, Platanus acerifolia, or any combination thereof.

The compositions of the present disclosure may further comprise a flavonoid. The flavonoid may be in an amount ranging from 0.001% to 0.1%. Flavonoids possess a wide range of biological and pharmaceutical activities, including antioxidant and anti-inflammatory activities. In certain embodiments, the flavonoid comprises catechin, epicatechin, rutin, luteolin, apigenin, kaempherol, myricetin, quercetin, quercitrin, naringin, naringenin, hesperetin, hesperidin, taxifolin, genistin, genistein, daidzein, cyanidin, apigenidin, tangeritin, fisetin, galangin, isorhamnetin, pachypodol, rhamnazin, pyranoflavonol, fluranoflavonol, eriodictyol, homoeriodictyol, taxifolin, gallocatechin, catechin 3-gallate, gallocatechin 3-gallate, epigallocatechin, epicatechin 3-gallate, epigallocatechin 3-gallate, theaflavin, thearubigin, proanthocyanidin, dephinidin, malvidin, pelargonidin, peonidin, petunidin, isoflavone, glycitein, isoflavane, isoflavandiol, isoflavene, coumestan, pterocarpan, myricitrin, phloridzin, or any combination thereof. In certain embodiments, the flavonoid comprises a citrus essential oil (e.g., Citrus reticulata) as disclosed in Y Yang, et al., J Food Sc 12 (217): 2840-2846, hereby incorporated by reference in its entirety, wherein monoterpene hydrocarbons comprise at least 75% or at least 85% of the total composition of the essential oil. Monoterpene concentration may be measured using the method as described in Njoroge et al. (Journal of Essential Oil Research 18:659, 2006) hereby incorporated by reference in its entirety.

In certain embodiments, compositions of the present disclosure may further comprise a sesquiterpene alcohol and/or monocyclic sesquiterpene. The sesquiterpenes may be in an amount ranging from 0.001% to 0.1% by weight of the composition. Sesquiterpenes possess a wide range of biological and pharmaceutical activities, including increasing skin hydration, antimicrobial activity, and antifungal activity. In certain embodiments, the sesquiterpenes may include nerolidol, an essential oil from Citrus aurantum var sp, bisabolol, patchoulol, alpha-santalol, zingiberene, or combinations thereof. In some embodiments, the sesquiterpene alcohol or monocyclic comprises nerolidol, an essential oil from Citrus aurantum (e.g., Citrus aurantium var. amata) and/or Citrus bigaradia, bisabolol, patchoulol, alpha-santalol, zingiberene, or combinations thereof. In certain implementations the sesquiterpene alcohol and/or monocyclic sesquiterpene is neroli oil.

The composition may further comprise a biofilm inhibiting agent. The biofilm inhibiting agent may be in an amount ranging from 0.001% to 0.16%. In certain embodiments, the biofilm inhibiting agent comprises Lamiaceae essential oil, Garcinia extract, Eurycoma longifolia extract, or any combination thereof. Examples of Lamiaceae essential oil include essential oil from Mentha spicata, Mentha pulegium, Mentha piperita, Mentha aquatica, Mentha arvensis, Mentha asiatica, Mentha australis, Mentha canadensis, Mentha cervina, Mentha citrata, Mentha crispata, Mentha dahurica, Mentha diemenica, Mentha laxiflora, Mentha, longifolia, Mentha requienii, Mentha sachalinensis, Mentha satureioides, Mentha suavenolens, Mentha vagans, Melissa officinalis, Monarda fistulosa, or any combination thereof. Garcinia plants are sources of antioxidant xanthones, which possess anti-microbial and anti-inflammatory properties. Examples of xanthones include y-mangostin, garcinone-D, gartanin, and smeathxanthone. Examples of Garcinia extract include extract from Garcinia mangostana, Garcinia travancorica, Garcinia cambogia, Garcinia kola, Garcinia zeylanica, Garcinia xanthochymus, or any combination thereof.

In certain implementations, compositions of the present disclosure further comprise a cell membrane active phytosterol. The phytosterol may be in an amount ranging from 0.001% to 0.1%. Phytosterols may inhibit the growth of pathogenic bacteria and have the potential to inhibit the activity of pore-forming toxins such as vaginolysin, leukotoxin, and other cholesterol dependent cytotoxins found in pathogenic strains of bacteria such as some Staphylococcus, Clostridium, and Gardnerella spp. In certain embodiments, the phytosterols comprise apigenin, β-sitosterol, campesterol, brassicasterol, stigmasterol, sitosterol, or any combination thereof. Examples of phytosterol sources include ginseng (Panax quinquefolium) seed extract, carrot, yam or coriander extract, ginger root extract, Mirabilis Jalap, or any combination thereof.

In certain embodiments, compositions of the present disclosure further comprise a prebiotic spice extract. The prebiotic spice extract may be in an amount ranging from 0.001% to 0.02% by weight of the composition. Exemplary prebiotic spice extracts include extract from ginger, black pepper, cayenne pepper, cinnamon, oregano, rosemary, turmeric, or any combination thereof.

The compositions of the present disclosure may also comprise a viscosity-increasing agent. Viscosity is a property of liquids that is closely related to the resistance to flow. It may be defined by Couette flow, which is the laminar flow of a viscous fluid in the space between two parallel plates, one of which is moving relative to the other. The flow is driven by virtue of viscous drag force acting on the fluid and the applied pressure gradient parallel to the plates.

The compositions may comprise one or more rheology agents. In some embodiments, the composition may comprise one or more non-cellulose based rheology agents. In some embodiments, the topical, isotonic composition comprises a rheology agent selected from poloxamers, polyacrylics (e.g. polyacrylic acids), polybutene, polycarbophil, polyvinyl alcohol, polyvinylpyrrolidone polymers, polyoxazoline polymers, and combinations thereof.

In some embodiments, the compositions of the present disclosure further comprise one or more humectants such as glycerin, hexylene glycol, arabinogalactan, caprylyl glycol, or combinations thereof. In certain embodiments, the compositions may comprise a humectant that is an extract of a plant from the genus Monotropa (e.g., Monotropa hypopitys) such as those described in JP App No 2009191075 A to Yamada, hereby incorporated by reference in its entirety.

In some embodiments, the compositions of the present disclosure further comprise one or more anti-inflammatory agents and/or one or more soothing agents. Specific anti-inflammatory and/or soothing agents include ICAM inhibitors (e.g., CD11a, ezrin (EZR), CD18, glycyrrhetinic acid, pyrrolidinedithiocarbamate), NFκB inhibitors (e.g., (heterocyclic thiazole, lipoic acid, efalizumab, 4-[(4- Methylphenyl)thio]thieno[2,3-c]pyridine-2-carboxamide, silibinin, stilbenes, (+)-epigalloylcatechin-gallate [(+)-EGCG]), cytokine inhibitors TSLP inhibitors, IL-25 inhibitors, IL-33 inhibitors, IL-1 inhibitors, TNF inhibitors (e.g., TNF-α inhibitors, TNF-β inhibitors), quercetin and isoforms thereof (e.g., isoquercetin, etc.), non-steroidal anti-inflammatory drugs (e.g., aspirin), and extracts from plants of the genus Vigna (e.g., Vigna caracalla), extracts of plants from the genus Rhododendron (e.g., Rhododendron aceae), extracts of plants from the subfamily Monotropaceae such as Allotropa virgate as disclosed in JP 2009191075 to T Yamada, hereby incorporated by reference in its entirety, and combinations thereof. In various embodiments, the anti-inflammatory agent and/or soothing may be present in an amount of from 0.0001 to 10% (e.g. from 0.001 to 5%) by weight of the composition

The extracts may be prepared by enzymatic extraction, solvent extraction, steam distillation, or any other method known in the art. In some embodiments, at least one of the topical compositions of the invention comprises an extract, obtained by steam distillation, of any of the forgoing plants and biological materials (each one being considered a distinct embodiment). In some embodiments, at least one of the topical compositions of the invention comprises an extract, obtained by extraction with water (e.g., basic, neutral, or acid), of any of the forgoing plants and biological materials (each one being considered a distinct embodiment). The water of extraction may further include a co-solvent miscible with water, including lower alcohols (e.g., C₁₋₆), such as methanol, ethanol, isopropanol, propanol, butanol (typically, ethanol). In some embodiments, at least one of the topical compositions of the invention comprises an extract, obtained by extraction with a solvent system comprising from 5-95% (v/v) or 10-90% (v/v) or 20-80% (v/v) or 40-60% (v/v) water (e.g., basic, neutral, or acid) and 5-95% (v/v) or 10-90% (v/v) or 20-80% (v/v) or 40-60% (v/v) ethanol, of any of the forgoing plants and biological materials (each one being considered a distinct embodiment). In some embodiments, at least one of the topical compositions of the invention comprises an extract, obtained by extraction with an organic solvent (e.g., non-polar, polar aprotic, or polar protic), of any of the forgoing plants and biological materials (each one being considered a distinct embodiment). Suitable solvents include hexane and other C₁₋₁₂ or C₅₋₈ hydrocarbons, lower alcohols, C₂₋₁₆ ethers (e.g., diethyl ether), C₃₋₁₂ esters (e.g., ethyl acetate), C₂₋₁₂ (e.g., acetone, butanone), carbon dioxide (liquid or supercritical) The biological extracts may be dried under vacuum or atmospheric pressure to remove water and solvents of extraction. The biological extracts may be dried by lyophilization. The biological extracts may be passed over activated carbon or charcoal and/or passed through filters and/or microfilters to remove bacteria and other biological materials.

In certain embodiments, compositions of the instant disclosure are formulated to have viscosity best suited for the target tissue (e.g., anogenital region) and to mimic the properties of normal genital fluids. For example, compositions formulated as gels applied to mucous membranes may be designed to have viscosity values consistent with or similar to normal mucus, and exhibiting non-Newtonian, shear-thinning (pseudoplastic) flow properties. Standardized methodology for quantitative comparisons of over-the-counter vaginal products' base features such as, stickiness, ropiness, peaking, rubberiness, thickness, smoothness, and slipperiness, are known in the art (Mahan et al., Contraception, 84:184, 2011). In some embodiments, compositions formulated as gels applied to mucous membranes may strengthen mucus quality and/or mucin coverage of the body surface. In certain implementations, the compositions may stimulate the production or proffer an acidic barrier in the urogenital and/or anogenital region with improved muco-adhesion, which may increase the bioavailability of one or more active components of the composition and result in a beneficial impact on the genital microbiome as disclosed in N Peppas, et al., J Biomater Sci Polym Ed. 20 (2209): 1-20, hereby incorporated by reference in its entirety and particularly in relation to muco-adhesive carrier development. In certain embodiments, the compositions of the present disclosure may include a specific buffer which allows for increased buffering capacity in the vagina at a pH range of 5-7.

For compositions applied to skin (such as the penis, vulva, perineum) or inside the vagina, a viscosity-increasing agent can be added in an amount that allows the composition to spread easily to form a thin layer when minimal physical pressure is applied, and to have adequate viscosity and shear-thinning properties so that the composition does not “run” off or out of the genital tissue upon topical application. Mucoadhesive formulations that are retained at the genital surface (e.g., vulvar, vaginal, penile, foreskin surface) for prolonged biological activity are known in the art (reviewed by Khutoryanskiy, Macromol. Biosci. 11:748, 2011; Brooks, Front. Chem. 3:65, 2015). Muco-adhesive formulas must have polymer compositions that actively admix and interact with physiologic mucin and mucus of secretions. Some common gelling agents do not intertwine with natural mucins and are therefore not muco-adhesive and are rapidly lost from the epithelium. In certain embodiments, compositions of the instant disclosure are formulated to have viscosity best suited for the target tissue (e.g., penile region) and to mimic the properties of normal genital fluids. Standardized methodology for quantitative comparisons of over-the-counter products may be based features such as, stickiness, ropiness, peaking, rubberiness, thickness, smoothness, and slipperiness, are known in the art.

Compositions of the present disclosure may comprise a viscosity-increasing agent in an amount ranging from, for example, 0.05% to 10% by weight of the composition. In certain embodiments, the viscosity enhancing agent comprises a tensioactive cellulose or gum. Tensioactive celluloses and gums can also act to emulsify and pull particles and essential oils into solution. In certain embodiments, additional surfactants, which may have a harsh effect on cells, are not needed or included in the compositions of the present disclosure. For example, the composition may be substantially free of surfactants such as having less than 1% or less than 0.1% or less than 0.01% surfactants by weight of the composition. In certain embodiments, the viscosity-increasing agent comprises guar gum, methylcellulose, ethylcellulose, ethyl methyl cellulose, ethyl hydroxyethyl cellulose, carboxymethylcellulose, hydroxypropylcellulose, hydroxyethyl methyl cellulose, hydroxypropylmethylcellulose (hypromellose), hydroxyethylcellulose, cetyl hydroxyethycellulose, hydroxypropyl guar gum glycosaminoglycans (e.g., hyaluronic acid), pullulan, nonionic triblock copolymers such as poloxamers, gelatins, alginates, carrageenan, and agar, or any combination thereof. In some embodiments, the compositions may comprise a viscosity-increasing agent comprising glycosaminoglycans (e.g., hyaluronic acid), polyacrylic acids, nonionic triblock copolymers such as poloxamers, gelatins, carrageenan, agar, and combinations thereof.

Compositions having a pH of 3.5 to 6, may be particularly suited for administration to a subject that is aged from adolescence to menopause/andropause, e.g., 18 years of age to 50 years of age, or a child at least one year old. In certain embodiments, an isotonic, topical composition for administration to a subject of more than 12 years of age (e.g., from 12 years of age to 50 years of age, more than 50 years of age, more than 60 years of age, more than 70 years of age, more than 80 years age, more than 90 years of age, more than 100 years of age) has a pH in the urogenital and/or anogenital region of from 3.8 to 4.8. In certain embodiments, an isotonic, topical composition for administration to a child at least one year old has a pH in the anogenital and/or urogenital region of 5. Topical, isotonic compositions having a pH of 4.8 to 7 are particularly suited for administration to an infant aged less than 12 months old, to a subject of 18 years of age to 50 years of age who is trying to conceive a child, or a senior of at least 60 years of age. In certain embodiments, a topical, isotonic composition for administration to an infant has a pH in the anogenital and/or urogenital region of 6.5. In certain embodiments, a topical, isotonic composition for administration to a subject of 18 years of age to 50 years of age who is trying to conceive a child has a pH in the anogenital and/or urogenital region of from 6.5 to 7.

Topical compositions of the present disclosure may be isotonic to the target genital fluids or tissues that they will contact. Tonicity is a measure of the effective osmotic pressure gradient (as defined by the water potential of two solutions) of two solutions separated by a semipermeable membrane. Tonicity is commonly used when describing the response of cells immersed in an external solution. In other words, tonicity is the relative concentration of solutions that determine the direction and extent of diffusion from a fluid across cell membranes in tissue. Blood normally has an osmotic pressure that corresponds to that of a 0.9% solution of sodium chloride. A composition (e.g., solution or gel) is considered isotonic when its tonicity matches that of the physiologic fluids it will contact. A composition is isotonic with a body fluid when the magnitude of the salts (ions) are equal between the composition and the physiologic fluid. Tonicity equilibrium is reached in physiologic fluids by water moving across cell membranes, but the salts and ions staying in their fluid of origin. A solution is isotonic with a living cell if there is no net gain or loss of water by the cell, or other changes in the cell ultrastructure, when it is in contact with said solution, even though individual water molecules may move freely across the cell membranes.

Hypertonic solutions cause a net movement of water out of the cells (as the water moves to create equilibrium with the high salt levels outside of the cell). This dehydration of the cell is concentration dependent and leads to osmotic stress which can increase reactive oxygen species, cause cytoskeletal rearrangement, and damage DNA and mitochondrial function within minutes of exposure. Most current genital products are hypertonic, resulting in epithelial cell and sperm death on contact. Hypotonic solutions cause a net flow of water into the cell and cause cell bursting and death. Some deviations of salt levels in physiologic fluids from the level found in blood and tissues may serve a purpose. For example, the lower osmolality of cervico-vaginal fluids that facilitates vaginal epithelial cell lysis and death as a part of normal vaginal function. In another example, the higher osmolality of semen can protect sperm cells from the lower osmolality of cervico-vaginal secretions following ejaculation in the vagina and admixing of fluids during vaginal intercourse.

Related to tonicity is osmosis, which is the movement of solvent across a semipermeable membrane from an area of higher solute concentration to an area of lower solute concentration to produce equilibrium. Osmotic pressure of a solution is the pressure that must be applied to stop the flow of solvent across a semipermeable membrane. In certain embodiments, the compositions of the present disclosure further comprise an osmolality adjusting agent to adjust the tonicity of the compositions. Exemplary osmolality adjusting agents include electrolytes, mono- or disaccharides, inorganic salts (e.g., sodium chloride, calcium chloride, potassium chloride, sodium sulfate, magnesium chloride), or a combination thereof. In some embodiments, an osmolality adjuster is glucose, sucrose, sodium chloride, potassium chloride, calcium chloride, sodium sulfate, magnesium chloride, dextrose, mannitol, or any combination thereof.

In certain embodiments, the osmolality range of the compositions disclosed herein ranges from 120 mOsm/kg to 450 mOsm/kg or from 240 mOsm/kg to 450 mOsm/kg. In certain embodiments, the osmolality of the compositions of the present disclosure is 120 mOsm/kg, 125 mOsm/kg, 130 mOsm/kg, 135 mOsm/kg, 140 mOsm/kg, 145 mOsm/kg, 150 mOsm/kg, 155 mOsm/kg, 160 mOsm/kg, 165 mOsm/kg, 170 mOsm/kg, 175 mOsm/kg, 180 mOsm/kg, 185 mOsm/kg, 190 mOsm/kg, 195 mOsm/kg, 200 mOsm/kg, 205 mOsm/kg, 210 mOsm/kg, 215 mOsm/kg, 220 mOsm/kg, 225 mOsm/kg, 230 mOsm/kg, 235 mOsm/kg, 240 mOsm/kg, 245 mOsm/kg, 250 mOsm/kg, 255 mOsm/kg, 260 mOsm/kg, 265 mOsm/kg, 270 mOsm/kg, 280 mOsm/kg, 285 mOsm/kg, 290 mOsm/kg, 295 mOsm/kg, 300 mOsm/kg, 305 mOsm/kg, 310 mOsm/kg, 315 mOsm/kg, 320 mOsm/kg, 325 mOsm/kg, 330 mOsm/kg, 335 mOsm/kg, 340 mOsm/kg, 345 mOsm/kg, 350 mOsm/kg, 355 mOsm/kg, 360 mOsm/kg, 365 mOsm/kg, 370 mOsm/kg, 375 mOsm/kg, 380 mOsm/kg, 385 mOsm/kg, 390 mOsm/kg, 395 mOsm/kg, 400 mOsm/kg, or 450 mOsm/kg.

In certain embodiments, the topical, isotonic composition is matched for tonicity (e.g., salt/ion levels) to the normal, physiological genital fluid pH (e.g., CVF, urethral secretions, semen, smegma) of the subject; genital tissue of the subject (e.g., vaginal mucosa, genital skin); or at an appropriate tonicity for the particular method of use (e.g., for use during fertile window in a subject or sexual dyad that is trying to conceive). In certain embodiments, the tonicity ranges from 125 mOsm/kg to 240 mOsmo/kg. Such embodiments match the hypotonic CVF which supports lysis of vaginal epithelial cells and vaginal “self-cleaning.” This cell lysis releases glycogen, which healthy genital microbiota utilize for growth and development. Such embodiments are ideal for delivery inside the vaginal canal. In other embodiments the tonicity ranges from 240 mOsm/kg to 280 mOsm/kg. Such embodiments match the tonicity of genital tissues and are ideal for contact with skin genital tissue surfaces. In a particular embodiment, the tonicity ranges from 280 mOsmo/kg to 450 mOsmo/kg to match the tonicity of semen as deposited in the vagina. In certain embodiments, tonicity may be expressed as mOsm/kg or mOsm/L. In certain embodiments, the osmolality and osmolarity values are substantially interchangeable.

In certain embodiments, the topical, isotonic composition further comprises a solvent (e.g., aqueous solvent, water) in an amount greater than 88% (e.g., ranging from 88% to 98%). In further embodiments, the solvent comprises water.

In certain embodiments, the topical, isotonic composition further comprises an additional therapeutic agent. The additional therapeutic agent may improve cell or tissue function or treat an underlying disease or disorder. In certain embodiments, the therapeutic agent is an antibiotic, anti-fungal agent, anti-viral agent, or any combination thereof. Exemplary anti-fungal agents include butoconazole nitrate, clotrimazole, miconazole nitrate, terconazole, tioconazole, econazole nitrate, efinaconazole, ketoconazole, luliconazole, naftifine hydrochloride, oxiconazole nitrate, sertaconazole nitrate, sulconazole nitrate, tavaborole, terbinafine, acyclovir, tenovir, zidovudine, stavudine, metronidazole, or a combination thereof. In some embodiments, the additional therapeutic agent is a vaccine (e.g., multivalent vaccine) to provide immunity against a viral or bacterial disease. Suitable vaccines include uropathogenic Escheri coli bacteria as disclosed in W Hopkins, et al., J Urol., 177 (2007): 1349-1353, hereby incorporated by reference in its entirety, and particularly in relation to the vaccine suppositories used in the study. In some embodiments, the vaccine may include a cholera vaccine such as those as disclosed in P Kozlowski, et al., Infection and Immunity 65 (1997): 1387-1394, which is hereby incorporated by reference in its entirety and particularly in relation to cholera vaccines, and vaccines containing killed Vibrio cholerae cells. In some embodiments, the vaccine may include a SARS-CoV-2 or COVID-19 vaccine. In some embodiments, the additional therapeutic agent may treat or prevent atrophic vaginitis. Suitable agents for the treatment or prophylaxis of atrophic vaginitis include hyaluronic acid, estrogens including estradiol-17β, conjugated estrogens, estradiol hemihydrate, dehydroepiandrosterone, estradiol acetate, selective estrogen receptor modulators, including bazedoxifene, cyclofenil, lasofoxifene, ormeloxifene, ospemifene, raloxifene, toremifene, and combinations thereof.

In certain embodiments, the additional therapeutic agent is a topical pain-relieving agent. Exemplary topical pain-relieving agents include lidocaine, benzocaine, novocaine, diphenhydramine, and pramoxine.

Other examples of therapeutic agents include hormones (e.g., estrogen, estradiol, estriol, estropipate, testosterone, progesterone, DHEA, testosterone, or a combination thereof), contraceptive agents (e.g., impairs sperm function, thickens cervical mucus, or both), agents that enhance vasodilation (e.g., In some embodiments, an agent that enhances vasodilation is L-arginine, niacin, nicotinamide, alprostadil, a phosphodiesterase inhibitor), erectile dysfunction treatment or erectile enhancement drugs (e.g., alprostadil, glyceryl trinitrate, sildenafil, tadalafil, vardenafil, avanafil, lodenafil, mirodenafil, udenafil, zaprinast, Icariin, benzamidenafil, dasantafil), and premature ejaculation drugs (e.g., selective serotonin reuptake inhibitors including sluoxetine, paroxetine, sertraline; tricyclic antidepressants including clomipramine).

Yet another example of a therapeutic agent is a skin conditioner or emollient.

In certain embodiments, the topical, isotonic compositions of the present disclosure further comprise at least one genital probiotic bacterial species or strain (e.g., belonging to the genus Lactobacillus). In certain embodiments, the probiotic bacterial species or strain is one having the ability to colonize the human vagina or penis. The adhesion of lactobacilli to the uroepithelium varies among species and strains, as shown by in vitro studies (Reid et al., J. Urol. 138:330, 1987), and may be mediated by glycoprotein and carbohydrate adhesins binding to glycolipid receptors (Boris et al., Infection and Immunity 66:1985, 1998). In some embodiments, a genital probiotic species is a species that is part of the genital microbiota (e.g., vagina or penis). In a specific embodiment, a genital probiotic species is selected from Lactobacillus acidophilus, Lactobacillus jensenii, Lactobacillus gasseri, Lactobacillus iners, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobaccillus reuteri, and Lactobacillus rhamnos, Streptococcus, non-pathogenic Prevotella species, Bacillus, or any combination thereof.

The topical, isotonic compositions of the present disclosure may further comprise additional pharmaceutical excipients. Pharmaceutically acceptable excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Compositions of the present disclosure can be formulated as a liquid, semi-solid, soap, gel, jelly, film, foam, cream, douche, ointment, lotion, spray, aerosol, suspension, emulsion, or paste. In certain embodiments, the compositions are formulated in a single-use or unit-dose format. Particularly in single-use compositions, the compositions may be free or substantially free (e.g., less than 1% w/w or less than 0.1% w/w) of preservatives.

In certain embodiments, topical, isotonic compositions of the present disclosure are integrated into a tampon, vaginal ring, cervical cup, diaphragm, condom, wipe, blanket, undergarment, or diaper. In certain embodiments, the topic, isotonic compositions are administered using a syringe, a roller ball, foam dispenser, spray bottle, aerosol dispenser, or pump dispenser.

In certain embodiments, compositions of the present disclosure are integrated into a microbiota sample collection and recovery system, such as a sterile swab or cyto-brush. In certain embodiments compositions of the present disclosure are used to facilitate the transplantation, storage, and cultivation of desirable gastrointestinal, vaginal, genital, reproductive tract, urinary tract, and/or anogenital microbiota from healthy donors for use in the treatment of microbial dysbiosis in affected recipients. For example, a vaginal rinse, using the compositions of the present disclosure may be collected from the vagina of healthy user and transferred to a subject with a dysbiotic microbiome of the anogenital and/or urogenital region, to promote beneficial microbiome transplantation. Such procedures are described in A Lev-Sagie, et al., Nature Medicine 25 (2019): 1500-1504, hereby incorporated by reference in its entirety. In another example, using the compositions of the present disclosure, a wipe may be used by a parent to collect beneficial genital microbiome species to transfer these via topical application to an infant born by Cesarean section.

In certain embodiments, compositions of the present disclosure may be used to add and facilitate the implantation process associated with tampons, reusable silicon devices such as menstrual cups for the control of natural menstrual flow in women or lochia post-partum and/or pelvic prolapse devices. Kits are also provided comprising a device for insertion into the vagina for control of natural menstrual flow such as a tampon and/or reusable silicon device, wherein the device is packaged in a composition of the present disclosure. For example, the compositions may be formulated with a pH-balanced gel for use in a tampon lubricant. These gels may contain a lactic acid buffer designed to maintain vaginal pH between 3.8 and 4.2 for 5 to 10 hours or 6 to 9 hours or 8 hours.

In certain embodiments, the composition is tailored to the individual recipient and donor profile to maximize the efficacy of the microbiota transplantation for each individual (e.g., the composition may be formulated for the production of specific bacteria in a user's microbiome). In certain embodiments, the composition would sustain beneficial microorganisms such as Lactobacillus acidophilus, Lactobacillus jensenii, Lactobacillus gasseri, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobaccillus reuteri, Lactobacillus rhamnosus, and combinations thereof. In addition, compositions may also sustain other bacterial species of the Leptotrichia, Leuconostoc, Pediococcus, Akkermansia, Streptococcus, Faecalibacterium and Weissella genera during the time interval between collection and implantation. In certain embodiments, compositions of the present disclosure may inhibit and/or minimize the growth of organisms that are or can become pathogenic including the species Lactobacillus iners, or species from the genus Prevotella, Eggerthellia, Gardnerella, Atopobium, Megasphaera, Mobiluncus, Mageeibacillus, Gemella, Veillonella Sneathia Clostridium, and combinations thereof. The growth inhibition and/or minimization may occur in microbial transplantation sample storage media.

The compositions may be formulated with thermoresponsive polymers which undergo a phase change exhibiting a sol-gel transition in response to body temperature, pH, and specific ions present in physiological environments. Such thermoresponsive polymers may prolong the residence time of the composition in the urogenital and/or anogenital region (e.g., vagina). Exemplary thermoresponsive polymers include poloxamers, styrene-butadiene block copolymer, polymethylacrylate, polybutylmethacrylate, plasticized polyvinylchloride, plasticized nylon, plasticized polyethyleneterephthalate, polyethylene, polyacrylonitrile, polytrifluoro chloroethylene, poly-4,4′-isopropylenediphenylene carbonate, polyethylenevinyl esters, polyvinylchloride-diethyl fumarate, and combinations thereof.

In various implementations, the compositions further comprise one or more sustained release polymers. Suitable sustained release polymers include sodium alginate (e.g., with barium chloride), barium chloride, poly-1-lysine, polyvinylamine, protamine sulfate, and combinations thereof.

Vaginal rings or pessaries are devices inserted in the vagina to achieve controlled release of the active composition. Medicated vaginal rings may be fabricated from Silastic® 382 medical grade elastomer. The most commonly used polymers for vaginal ring are ploy (dimethylsiloxane) or silicon devices, or ethylene vinyl acetate. Additionally, biodegradable polymers, such as polycaprolactone have been used to prepare these devices. These are generally polymeric rings in which the drug (e.g., borneol, pharmaceutically acceptable salts thereof, or prodrugs of any of the foregoing such as bornyl acetate, prebiotic oligosaccharide, metal co-factor) and/or composition is homogeneously dispersed. In order to achieve constant release, two types of system are typically developed for vaginal rings: sandwich and reservoir type. In the sandwich type, a narrow layer of the active components is placed between non-medicated central core and non-medicated outer band. In reservoir type, central core having the active components is encapsulated with drug-free polymer layer (e.g., hypromellose).

The compositions and/or active ingredients (e.g., borneol, pharmaceutically acceptable salts thereof, or prodrugs of any of the foregoing such as bornyl acetate, prebiotic oligosaccharide, metal co-factor) may also be formulated in vaginal suppositories, ovules, or pessaries. In these embodiments, the suppository, ovule, or pessary may be formulated with mixture of synthetic triglycerides (e.g. Witepsol H-15), hypromellose, polyethylene glycol polymers (e.g. PEG 1000, 4000), Sorbitan monostearate (Span 60), PEG-35 Castor Oil, PEG 400, PEG 3350, cocoa butter, polyethylenimine, mixtures of mono/di and triglycerides (e.g. Suppocire, Ovucire), Agar, Propylene glycol, Hypromellose, gelatin, glycerin, cocoa butter, bees wax, Polyoxyl 40 stearate, Polyvinyl alcohol, hydroxyethyl methacrylate, polyacrylic acid, polyoxyethylene, and combinations thereof.

In some embodiments, the composition may be formulated as an emulsion. The emulsion may be, for example, a water-in-oil, oil-in-water, silicone-in-water, water-in-silicone, polyol-in-oil, oil-in-polyol, glycerin-in-oil, oil-in-glycerin, silicone-in-glycerin, glycerin-in-silicone, silicone-in-polyol, or polyol-in-silicone emulsion. The topical isotonic formulations of the present disclosure may be used as the aqueous phase (e.g., the internal phase, the discontinuous phase) of an emulsion. In an emulsion, will be understood that the weight percentage of components used herein (e.g., borneol, pharmaceutically acceptable salts thereof, or prodrugs of any of the foregoing such as bornyl acetate, prebiotic oligosaccharide, metal co-factor) refers to the weight percentage of the composition (and not the weight percentage of the phase itself). The nonaqueous phase may comprise saturated (lauric, myristic and capric acid) and unsaturated fatty acids (oleic acid, linoleic acid and linolenic acid), surfactants, and co-surfactants. Exemplary components in emulsions include sorbitan oleate (Span80), Sorbitane trioleate (Span85), polyethylene glycol sorbitan monolaurate (Tween80), Soybean oil, squalene, lecithin, oleic acid, medium chain triglyceride, Polyoxyl 40 Hydrogenated Castor Oil, Polyoxyl 35 castor oil, Glycerol, Propylene glycol, and combinations thereof.

In certain implementations, the emulsion is a Pickering emulsion. Typically, Pickering emulsions are emulsions stabilized by solid particles including nanocellulose, graphene oxide, carbon nanotube, carbon lamp black, laponite, montmorillonite, silica nanoparticles, calcium carbonate, titanium dioxide, magnetic particles, polymer particles, and combinations thereof.

In some embodiments, the compositions or active ingredients (e.g., borneol, pharmaceutically acceptable salts thereof, or prodrugs of any of the foregoing such as bornyl acetate, prebiotic oligosaccharide, metal co-factor) are formulated as a tablet. For example, the composition may be in tablet form such as chitosan and/or sodium alginate based bio-adhesive tablets. In certain embodiments, the table further comprises a mucoadhesive. The compositions or active ingredients (e.g., borneol, pharmaceutically acceptable salts thereof, or prodrugs of any of the foregoing such as bornyl acetate, prebiotic oligosaccharide, metal co-factor) may be formulated as vaginal bioadhesive tablets. Vaginal bioadhesive tablets may comprise hydroxypropyl cellulose, polyacrylic acid, Carbopol-934, and combinations thereof.

In some embodiments, the compositions or active ingredients (e.g., borneol, pharmaceutically acceptable salts thereof, or prodrugs of any of the foregoing such as bornyl acetate, prebiotic oligosaccharide, metal co-factor) are formulated as liposomes (e.g., vaginal liposomes). Vaginal liposomes include lecithin-based liposomes which may incorporate bio-adhesive carbopol hydrogels. In some embodiments, the composition comprises a thermo-sensitive gel of poloxamers 407 and 188 and are in the form of active ingredients (e.g., borneol, pharmaceutically acceptable salts thereof, or prodrugs of any of the foregoing such as bornyl acetate, prebiotic oligosaccharide, metal co-factor) loaded cationic liposomes.

The compositions (e.g., gels), applicators, and/or kits of the present disclosure may be subjected to ozone sterilization. Wherein embodiments of the present disclosure may be exposed to ozone gas due to its oxidative potential. The possibility to alter different process parameters (e.g., time of exposure, gas concentration, humidity) allows the sterilization protocol to be adapted to different types of material.

The compositions (e.g., gels), applicators, and/or kits of the present disclosure may be subjected to one or more sterilization techniques such as steam heat sterilization, filtration sterilization, gamma irradiation sterilization, e-beam sterilization, or combinations thereof. These sterilization techniques may exclude, kill, or reduce the number of microorganisms present in the final composition. Various permutations of these sterilization techniques (e.g., time of exposure, pore size, radiation dose, temperature) may allow the sterilization protocol to be adapted to different materials in the compositions, devices, systems, applicators, kits, and sensors described herein.

Exemplary formulations according to the present disclosure are provided in Tables 1-31 and throughout the examples. In some embodiments, the compositions may comprise at least the pH adjusting agent, a buffer, bornyl acetate, a metallic cofactor and a prebiotic oligosaccharide. The remaining components may be optionally present in the amounts indicated.

TABLE 1

| Ingredient Name | % by wt. |
|---|---|
| Water | q.s. |
| pH adjusting agent (e.g., NaOH, lactic acid) | Adjusted to acidic pH |
| | (e.g., pH from 3.8 to |
| | 6.8, from 4 to 4.5, |
| | from 3.8 to 6, pH |
| | from 4 to 5, pH from |
| | 5 to 6) |
| Preservative (e.g., sodium benzoate, sodium | Less than 0.1 |
| dehydroacetate) | |
| Buffering agent (e.g., monosodium phosphate, | 0.001-10 |
| disodium phosphate, gluconolactone, combinations | |
| thereof) | |
| Tonicity agent (e.g., sodium chloride) | 0.001-10 |
| Prebiotic oligosaccharide | 0.01-1 |
| Essential oil comprising bornyl acetate (e.g., Abies | 0.01-10 |
| sibirica (Siberian fir)) | |
| Rheology modifier (e.g., hypromellose, | 0.05-20 |
| hydroxypropyl guar gum, combinations thereof) | |
| Humectant (e.g., arabinogalactan) | Less than 0.1 |
| Biofilm inhibiting agent (e.g., Mentha spicata | 0.01-0.5 |
| extract) | |
| Flavonoid (e.g., Citrus reticulata) | 0.001-0.1 |
| Sesquiterpene alcohol | 0.001-0.1 |
| Metallic co-factor (e.g., Manganese chloride) | 0.001-1 |

TABLE 2
Formula A

| Ingredient Name | % by wt. |
|---|---|
| Purified water, USP | q.s. |
| Sodium hydroxide | To pH 4.5 |
| Monosodium phosphate | 0.483 |
| Sodium chloride | 0.100 |
| Lactulose | 0.050 |
| Abies sibirica (Siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate). For example, in this embodiment the composition may comprise 0.003 % (w/w) MnCl (MW = 125.9 g/mol) as added from MnCl₂•4H₂O (MW = 197.9 g/mol). Therefore, 0.00472 g hydrate were added per 100 g of formulation.

TABLE 3
Formula B

| Ingredient Name | % by wt. |
|---|---|
| Purified water, USP | q.s. |
| Sodium hydroxide | To pH 6.8 |
| Monosodium phosphate | 0.483 |
| Sodium chloride | 0.100 |
| Lactulose | 0.050 |
| Abies sibirica (Siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 4
Formula C

| Ingredient Name | % by wt. |
|---|---|
| Purified water, USP | q.s. |
| Sodium hydroxide | To pH 4.5 |
| Disodium phosphate | 0.531 |
| Lactic acid | 0.500 |
| Sodium chloride | 0.0500 |
| Lactulose | 0.0500 |
| Abies sibirica (Siberian fir) | 0.0200 |
| Hypromellose | 0.6000 |
| Gluconolactone | 0.5600 |
| Hydroxypropyl guar gum | 0.4000 |
| Arabinogalactan | 0.2000 |
| Mentha spicata | 0.0100 |
| Citrus paradisi | 0.0100 |
| Sodium benzoate | 0.1900 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride

TABLE 5
Formula D

| Ingredient Name | % by wt. |
|---|---|
| Purified water, USP | q.s. |
| Sodium hydroxide | To pH 6.8 |
| Disodium phosphate | 0.531 |
| Lactic acid | 0.500 |
| Sodium chloride | 0.0500 |
| Lactulose | 0.0500 |
| Abies sibirica (Siberian fir) | 0.0200 |
| Hypromellose | 0.6000 |
| Gluconolactone | 0.5600 |
| Hydroxypropyl guar gum | 0.4000 |
| Arabinogalactan | 0.2000 |
| Mentha spicata | 0.0100 |
| Citrus paradisi | 0.0100 |
| Sodium benzoate | 0.1900 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride

TABLE 6
Formula E

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Sodium benzoate | 0.125 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 7
Formula F

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Lactobacillus Ferment | 1.750 |
| Sodium dehydroacetate | 0.056 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 8
Formula G

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Lactobacillus Ferment | 2.000 |
| Sodium dehydroacetate | 0.075 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride

TABLE 9
Formula H

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Abies sibrica | 0.020 |
| Hypromellose | 0.600 |
| Gluconolactone | 0.500 |
| Hydroxypropyl guar gum | 0.400 |
| Arabinogalactan | 0.050 |
| Mentha spicata | 0.010 |
| Neroli oil | 0.010 |
| Manganese chloride* | 0.005 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 10
Lubricant, pH 4.5, osmolality 150 mOsm/kg

| Ingredient Name | % by wt. |
|---|---|
| Purified water, USP | 96.1422 |
| Disodium phosphate | 0.39825 |
| Lactic Acid | 0.1425 |
| Lactulose | 0.05 |
| Abies sibirica (Siberian fir) | 0.02 |
| hydroxyethyl cellulose | 0.8 |
| Oleuropein | 0.02 |
| gluconolactone | 1.00 |
| hydroxypropyl guar gum | 0.4 |
| Arabinogalactan | 1.00 |
| Mentha spicata | 0.0075 |
| Citrus reticulata | 0.0075 |
| Juniperus communis | 0.0075 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate). For example, in this embodiment the composition may comprise 0.0046% (w/w) MnCl₂ (MW = 125.9 g/mol) as added from MnCl₂•4H₂O (MW = 197.9 g/mol). Therefore, 0.00723 g hydrate were added per 100 g of formulation.

TABLE 11
Foaming Gel, pH 4.5, osmolality 120 mOsm/kg

| Ingredient Name | % by wt. |
|---|---|
| Deionized Water, USP | 93.840 |
| Manganese chloride* | 0.008 |
| Lactulose | 0.05 |
| Abies sibirica (Siberian fir) | 0.02 |
| Cetyl Hydroxyethylcellulose | 0.5 |
| Oleuropein | 0.02 |
| sodium cocoyl glutamate | 2.00 |
| Sodium Lauroamphoacetate | 2.00 |
| Arabinogalactan | 1.00 |
| disodium phosphate | 0.39825 |
| Lactic Acid | 0.1425 |
| Mentha spicata | 0.008 |
| Citrus reticulata | 0.005 |
| Juniperus communis | 0.008 |

*The indicated weight percentage of metallic co-factor (i.e., magnesium chloride) may be achieved by adding the appropriate amount of a hydrate of the metallic salt (e.g., magnesium chloride (II) tetrahydrate).
†The indicated weight percent of Cocamidopropyl betaine is the weight percent from a 36% solutions of cocamidopropyl betaine may be added to each solution.

TABLE 12
Wipe Formula, pH 6.5 (infant 0-12 mo) or pH 5
(child > l yr), 180 mOsm/kg

| Ingredient Name | % by wt. |
|---|---|
| Deionized Water, USP | 94.861 |
| Lactulose | 0.100 |
| Abies sibirica (Siberian fir) | 0.020 |
| Cetyl Hydroxyethylcellulose | 0.500 |
| Oleuropein | 0.020 |
| Cocamidopropyl Betainet | 2.100 |
| Arabinogalactan | 0.900 |
| Citric Acid | 0.098 |
| disodium phosphate | 0.386 |
| Citrus reticulata | 0.010 |
| Manganese chloride* | 0.0046 |
| sodium benzoate | 0.3000 |
| gluconolactone | 0.7000 |

*The indicated weight percentage of metallic co-factor (i.e., magnesium chloride) may be achieved by adding the appropriate amount of a hydrate of the metallic salt (e.g., magnesium chloride (II) tetrahydrate).
†The indicated weight percent of Cocamidopropyl betaine is the weight percent from a 36% solution of cocamidopropyl betaine may be added to each solution.

TABLE 13
Lubricant, pH 6.8, osmolality 340 mOsm/kg

| Ingredient Name | % by wt. |
|---|---|
| Purified Water, USP | 95.67 |
| disodium phosphate | 0.70 |
| Lactic Acid | 0.18 |
| Lactulose | 0.03 |
| sodium chloride | 0.15 |
| Raffinose | 0.02 |
| Abies alba | 0.01 |
| hydroxyethyl cellulose | 0.80 |
| Oleuropein | 0.02 |
| Hydantoin | 1.00 |
| hydroxypropyl guar gum | 0.40 |
| Arabinogalactan | 1.00 |
| Monarda Fistulosa | 0.01 |
| Citrus reticulata | 0.01 |
| Rosmarinus officinalis | 0.00 |
| Manganese chloride* | 0.01 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 14
Douche formulation

| Ingredient Name | % by wt. |
|---|---|
| Deionized Water, USP | 98.019 |
| Monosodium Phosphate, anhydrous | 0.483 |
| Sodium Chloride | 0.100 |
| Lactulose | 0.050 |
| Abies sibirica (Siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lactic Acid 10% | Titrate to pH |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 15
Douche formulation, pH 4.5

| Ingredient Name | % by wt. |
|---|---|
| Deionized Water, USP | 99.061 |
| Monosodium Phosphate, anhydrous | 0.241 |
| Sodium Chloride | 0.200 |
| Lactulose | 0.050 |
| Abies sibirica (Siberian fir) | 0.020 |
| hypromellose | 0.400 |
| Mentha spicata (spearmint) | 0.015 |
| Neroli Oil | 0.010 |
| Manganese chloride* | 0.003 |
| Lactic acid 10% | Titrate To pH |
| | 4.5 |

*The indicated weight percentage of metallic co-factor (i.e., magnesium chloride) may be achieved by adding the appropriate amount of a hydrate of the metallic salt (e.g., magnesium chloride

TABLE 16
Foaming Gel formulation, pH 4.6

| Ingredient Name | % by wt. |
|---|---|
| Deionized Water, USP | 97.3375 |
| Sodium chloride | 0.1000 |
| Lactulose | 0.0500 |
| Rosemary essential oil | 0.0500 |
| Abies sibirica (Siberian fir) | 0.0100 |
| Hypromellose | 0.3000 |
| Green tea polyphenols | 0.0200 |
| Arabinogalactan | 0.1000 |
| Mentha spicata (spearmint) | 0.0200 |
| Neroli Oil | 0.0100 |
| Manganese chloride* | 0.0025 |
| Sodium Cocyl Isethionate | 1.0000 |
| Cocamidopropyl Betainef | 1.0000 |

*The indicated weight percentage of metallic co-factor (i.e., magnesium chloride) may be achieved by adding the appropriate amount of a hydrate of the metallic salt (e.g., magnesium chloride (II) tetrahydrate).
†The indicated weight percent of Cocamidopropyl betaine is the weight percent from a 36% solution of cocamidopropyl betaine may be added to each solution.

TABLE 17
Lubricant pH 4.5

| Ingredient Name | % by wt. |
|---|---|
| Purified Water, USP | 95.997 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lactobacillus Ferment | 2.000 |
| Sodium dehydroacetate | 0.075 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 18
Lubricant pH 4.5

| Ingredient Name | % by wt. |
|---|---|
| Purified Water, USP | 95.072 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lactobacillus Ferment | 3.000 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 19
Lubricant pH 4.0

| Ingredient Name | % by wt. |
|---|---|
| Purified Water, USP | 97.922 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Sodium dehydroacetate | 0.150 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 20
Lubricant pH 4.0

| Ingredient Name | % by wt. |
|---|---|
| Purified Water, USP | 97.772 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Glyceryl caprylate | 0.300 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 21
Lubricant pH 4.5

| Ingredient Name | % by wt. |
|---|---|
| Purified Water, USP | 97.672 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Euxyl K 903 | 0.400 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 22
Lubricant pH 4.5

| Ingredient Name | % by wt. |
|---|---|
| Purified Water, USP | 97.872 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve BDHA | 0.200 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 23
Lubricant pH 4.5

| Ingredient Name | % by wt |
|---|---|
| Purified Water, USP | 97.072 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve WpH-LO | 1.000 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 24
Lubricant pH 4.0

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Linatural-Ultra 3 | 0.500 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 25
Lubricant pH 4.5

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Linatural MBS-1 | 0.500 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 26
Lubricant pH 4.5

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve SSB | 0.500 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 27
Lubricant pH 4.5

| Ingredient Name | % by wt. |
|---|---|
| Purified Water, USP | 97.947 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Sodium Benzoate | 0.125 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 28
Lubricant/Tampon Insertion Gel (TIG) Formulation

| | No | |
|---|---|---|
| | Preservative | |
| Ingredient Name | % By Wt. | Preservative |
| Purified Water, Usp | 97.465 | 97.315 |
| Monosodium Phosphate | 0.240 | 0.240 |
| Sodium Chloride | 0.450 | 0.450 |
| Lactulose | 0.050 | 0.050 |
| Abies Sibirica | 0.020 | 0.020 |
| Hypromellose | 1.200 | 1.200 |
| Gluconolactone | 0.500 | 0.500 |
| Arabinogalactan | 0.050 | 0.050 |
| Mentha Spicata | 0.010 | 0.010 |
| Citrus Aurantium Var. Amara | 0.010 | 0.010 |
| Manganese Chloride* | 0.005 | 0.005 |
| Sodium Dehydroacetate | | 0.1500 |
| Sodium Hydroxide | Titrate To | Titrate To |
| | pH 4.0 | pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 29
Cleanser 1

| Ingredient Name | % by Wt. |
|---|---|
| Deionized Water | 88.8125 |
| Sodium Chloride | 0.6000 |
| Lactulose | 0.0500 |
| Rosmarinus Officinalis | 0.0250 |
| Abies Sibirica | 0.0100 |
| Poloxamer 188 | 10.0000 |
| Arabinogalactan | 0.1000 |
| Gluconolactone | 0.2500 |
| Mentha Spicata | 0.0200 |
| Citrus Aurantium Var. Amara | 0.0100 |
| Manganese Chloride* | 0.0025 |
| Sodium Dehydroacetate | 0.12 |
| Lactic Acid | Titrate To pH 5.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 30
Cleanser 2

| Ingredient Name | % by Wt. |
|---|---|
| Deionized Water | 95.6325 |
| Sodium Chloride | 0.7500 |
| Raffinose | 0.0500 |
| Rosmarinus officinalis | 0.0250 |
| Abies sibirica | 0.0100 |
| Phenethyl Alcohol | 0.1500 |
| Propanediol | 0.7500 |
| Pentylene Glycol | 0.6000 |
| Arabinogalactan | 1.0000 |
| Mentha Spicata | 0.0200 |
| Citrus Aurantium Var. Amara | 0.0100 |
| Manganese Chloride* | 0.0025 |
| Sodium Cocoyl Glutamate (37.5% Active) | 0.50 |
| Sodium Lauroamphoacetate (30%) | 0.50 |
| Lactic Acid | Titrate To pH 5.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 31
Formulation 061819C, pH 6.8

| Ingredient Name | % by wt. |
|---|---|
| Purified Water, USP | 96.8684 |
| Hypromellose | 0.6 |
| Disodium phosphate, anhydrous | 0.531 |
| Gluconolactone | 0.5 |
| Lactic acid | 0.5 |
| Hydroxypropyl guar gum | 0.4 |
| Sodium hydroxide | 0.206 |
| Arabinogalactan | 0.2 |
| Sodium chloride | 0.1 |
| Lactulose | 0.05 |
| Abies sibirica | 0.02 |
| Citrus paradisi | 0.01 |
| Mentha spicata (spearmint) | 0.01 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate).

It will be understood that components may have multiple purposes as those described herein. For example, citric acid may be considered a pH modifying agent and a buffering agent. Many of the components described in these tables are optional.

In Vitro and In Vivo Activity

(1) Evaluating Genital Microbiota

To assess whether topical, isotonic compositions of the present disclosure are harmful to the genital microbiota, testing of normal genital microbiota may be performed using methods known in the art.

The effect of any composition disclosed herein on genital microbiota (e.g., Lactobacillus species) may be determined by measuring minimal inhibitory concentration, the lowest concentration which prevents visible growth of a microorganism after overnight culture, or minimal microbicidal concentration, the lowest concentration required to reduce the viability of a culture by, for example, more than 99% or more than 99.9% or more than 99.99%.

In some embodiments, the effect of any composition disclosed herein on genital microbiota (e.g., Lactobacillus species) may be determined by DNA extraction and 16S rRNA gene sequencing and operational taxonomic units (OTUs) assignment and community states (CST) of vaginal microbiome can be defined using Jensen-Shannon divergence and Ward linkage hierarchical clustering following administration, as disclosed in X Hong, et al., PeerJ 7 (2019): e8131, which is hereby incorporated by reference in its entirety.

The following signs and symptoms are all associated with genital microbiome function and may be used to assay effectiveness of the products. Vaginal infection is highly correlated with Lactobacillus dominance (or loss thereof) of the genital microbiome. Infection may be determined by examining a wet mount smear in potassium hydroxide for detection of candidiasis and in saline for detection of motile trichomonas and clue cells. Bacterial vaginosis (BV) is associated with loss of Lactobacillus dominance as well. Women with BV have dysbiosis of the genital microbiome. BV diagnosis may be assessed according to Amsel clinical criteria or Nugent testing or H₂O₂ and leukocyte esterase levels. Typically, a healthy vaginal microbiome will have lower levels of BV or other infections. BV diagnostic tests include: Nugent score, Amsel criteria, clue cell presence, whiff test and newer diagnostic tests (Aptima Bacterial Vaginosis and Aptima Candida/Trichomonas Vaginitis Assays). The ‘Amsel's criteria’ requires that three of the following four criteria be met for BV: first, a vaginal pH of greater than pH 4.5; second, the presence of clue cells in the vaginal fluid; third, a milky, homogeneous vaginal discharge; and finally, the release of an amine (fishy) odor after addition of 10% potassium hydroxide to the vaginal fluid. Suitable protocols for laboratory diagnosis of BV include those disclosed in D. Money. Can J Infect Dis Med Microbiol 16 (2005): 77-79, hereby incorporated by reference in its entirety, and specifically in relation to BV diagnosis protocols.

Vaginal secretion measurements after use of the product may also be measured using a colorimetric or other assay for increased d-lactic acid and/or amylase as indicators of healthy microbiome as disclosed in J Leizer, et al., Reprod Sci. 25 (2018):854-860, or D Nasioudis et al., Reprod. Sci 22 (2015): 1393-1398.

Lack of vaginal Lactobacillus dominance has also been linked to high risk HPV infection and persistence, genital herpes, and HIV infections. Measuring STD levels in patients may help determine efficacy of the gel. Several reports have shown that anti-HIV levels in CVF are higher in women with healthy genital microbiomes as illustrated in M Torcia, Int J Mol Sci 20 (2019): E266, S Placios et al., Minerva Ginecol 70 (2018): 138-143, and R Hemalatha et al., Indian J Med Res 138 (2013): 354-359, each of which is hereby incorporated by reference in its entirety.

pH of the body including vaginal pH or penile pH may be measured by litmus paper, vaginal glove, electrode, or nanosensor as disclosed in U.S. Pat. No. 10,436,745, hereby incorporated by reference in its entirety and particularly in relation to pH nanosensors. These measurements may be used to assess bothersome vaginal symptoms and infection. For example, elevated vaginal pH is relatively sensitive for detecting BV and dysbiosis in women.

Vaginal bothersome symptom manifestation may be increased in women without Lactobacillus dominance. Vaginal symptom level and sexual function can be tracked following product use using published tools as disclosed in B Ettinger, et al., Menopause 5 (2008): 885-889, hereby incorporated by reference in its entirety. Furthermore, vaginal metabolomics can be used to determine genital microbiome health, as disclosed in T. Nelson, Front Physiol 6 (2015): 253, hereby incorporated by reference in its entirety.

Biogenic amines, amino acids, and metabolites are also biomarkers of BV and dysbiosis, as they facilitate the outgrowth of BV-associated taxa by (i) amino-acid decarboxylation that consumes intracellular hydrogen ions and change bacterial acid resistance; (ii) limiting the growth and resistance of host immunology to urogenital pathogens; and iii) being correlated with numerous host disease states, including STDs, cancer and dementias. To measure, samples may be eluted from swabs in sterile molecular water and subjected to both liquid and gas chromatography mass spectrometry.

The presence of pathogenic bacteria can also be assessed by measuring the concentration of endotoxins, lipopolysaccharides (LPS), and quantity of pathogenic bacteria in washes obtained from subjects following the use of compositions of the present disclosure (see., e.g., Aroutcheva et al., Anaerobe 14:256, 2008). High LPS concentrations create a toxic vaginal environment causing epithelial and gamete (e.g., sperm) damage. Even very low levels of LPS (e.g., 0.1 μ/mg) rapidly impact sperm function (see, Li et al., Tohoku Journal of Experimental Medicine 238:105, 2016, incorporated herein by reference in its entirety). Since adenosine triphosphate (ATP) is present in all microorganisms, such as bacteria, measuring the presence of ATP indirectly indicates the presence of bacteria. Many ATP detection methods utilize bioluminescence to determine the presence of ATP by first exposing a sample to an ATP-releasing agent (e.g., lysis buffer) and an ATP-activated light-producing substrate and enzyme (e.g., luciferin and luciferase). The amount of ATP may be quantified by measuring the light produced by the enzymatic reaction which is in relative light units (RLU). The light may be detected at an appropriate wavelength depending on the specific ATP assay (e.g., 525 nm to 640 nm). A variety of luciferase assays and their luminogenic reagents and conditions are known in the art, and may be readily used or adapted for use herein. See, e.g. Eed, H R et al., “Bioluminescence-Sensing Assay for Microbial Growth Recognition,” Journal of Sensors, vol. 2016, Article ID 1492467, 5 pages, 2016. https://doi.org/10.1155/2016/1492467; Mempin, et al. Release of extracellular ATP by bacteria during growth. BMC Microbiol 13, 301 (2013) doi:10.1186/1471-2180-13-301Fan F, Wood K V, “Bioluminescent assays for high-throughput screening”. Assay Drug Dev Technol. 5(1): 127-36, 2007; Meisenheimer, et al., “Luminogenic enzyme substrates: The basis for a new paradigm in assay design,” Promega Notes 100:22-26, 2008 at http://www.promega.com/pnotes/100/16620_22/16620_22. pdf.

Other methods that may be used to assess changes in genital microbiota following exposure to a topical, isotonic composition of this disclosure include performing 16S rRNA gene sequencing, shotgun metagenomic gene sequencing, microbial/host metabolomic profiling, and 3^{rd} generation sequencing utilizing nanopore DNA sequencing (see, e.g., Romero et al., Microbiome 2:4, 2014; Macklaim et al., Microb. Ecol. Health Dis. 26:27799, 2015), species specific quantitative PCR (Zozaya-Hinchliffe et al., J. Clin. Microbiol. 48:1812, 2010, each of which is incorporated herein by reference in its entirety), and phylogenetic microarrays (Paliy and Agans, FEMS Microbiol. Ecol. 79:2, 2012; Chen et al., Nat Commun. 8:875, 2017, each of which is incorporated herein by reference in its entirety) using bacterial samples obtained from subjects (e.g., washes, swabs).

In certain embodiments, the compositions of the present disclosure do not reduce normal genital microbiota population (e.g., L. crispatus, L. gassseri, L. jensenii, L. acidophilus, or any combination thereof) by more than 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% when exposed to a concentration or amount of the composition that is to be used in vivo on a subject.

In certain embodiments, the compositions of the present invention do not interfere with acid-producing bacterial growth and functional medium acidification of fluids or solutions of the genital microbiome (e.g., Lactobacillus spp) (see, Boskey et al., Infect Immun. 67: 5170, 1999, incorporated herein by reference in its entirety). For example, the composition may not interfere during in vivo application to a subject as described herein.

In certain embodiments, the compositions of the present disclosure do not promote the growth of pathogenic bacteria of the anogenital region by more than 1%, 2%, 3%, 4%, 5%, 6%, 7%, 8%, 9%, 10%, 11%, 12%, 13%, 14%, 15%, 16%, 17%, 18%, 19%, 20%, 25%, 30%, or 35%. In certain embodiments, the pathogenic bacteria are selected from pathogenic strains of Prevotella, Eggerthellia, Gardnerella, Atopobium, Megasphaera, Mageeibacillus, Mobiluncus, Bacteroides, Peptostreptococcus, Fusobacterium, Veillonella, Porphyromonas and Eubacterium.

(2) Evaluating Genital Irritation, Inflammation, and Cell Death

Irritation, inflammation, or cell death of genital tissues, such as the vaginal mucosa or penile foreskin cells, can be assessed in vitro or in vivo using human or animal vaginal-ectocervical, urethral, or foreskin tissue explants; vaginal, cervical or vulvar cell monolayers; penile epithelium or urethral epithelium; skin cell monolayers; slug mucosal irritation assays; or other equivalent methods. In order to predict the safety of the composition of the disclosure, instead of using animal testing (e.g., rabbit vaginal irritation (RVI) test, guinea pig maximization test (GPMT), acute systematic toxicity tests using rabbits or mice), in vitro methods based on human reconstructed tissue models may be performed. In one embodiment, a non-animal vaginal irritation method may be used to evaluate irritation of the isotonic composition disclosed herein. Briefly, a non-clinical assessment model (NAM), such as commercially available human reconstructed tissue models (e.g., EpiVaginal™ (MatTek Corporation; Ashland, Mass.); human vaginal epithelium (HVE) (SkinEthic (Lyon, France)) may be used for testing. See, e.g., Costin, G-E., et al. “Qualification of a non-animal vaginal irritation method admitted as nonclinical assessment model (NAM) in the Incubator Phase of the United States Food and Drug Administration (US FDA) Medical Devices Development Tool (MDDT).” Toxicology in Vitro 62 (2020): 104680, which is incorporated herein by reference in its entirety and particularly with respect to the NAMs.

For example, the slug mucosal irritation assay (SMI) is a sensitive system to detect even mild mucosal irritation potency (Adriaens et al., Sex. Transm. Dis. 35:512, 2008, incorporated herein by reference in its entirety). The SMI assay uses slugs (Arlon lusitanicus) as the test organism. The body wall of slugs consists of a mucosal surface comprising mucus secreting cells covering a sub epithelial connective tissue. Slugs that are placed on an irritant substance will actively produce mucus as a protective mechanism from noxious agents. Additionally, tissue damage of the slug's surface results in the release of proteins and enzymes. The protein concentration in the collected samples is determined with a protein quantitation kit. A composition of the present disclosure is considered non-irritating if it does not cause an increased production of mucus, or an increased release of proteins and enzymes as compared to a negative control.

Human, organotypic vaginal-ectocervical tissue models produced from normal human-derived vaginal-ectocervical epithelial cells may also be used to assess irritation of topically applied products, as can monolayers of cervical or vaginal epithelium (Ayehunie et al., Toxicology 279:130, 2011; Ayehunie et al., 2007, Toxicology 279:130, 2007; Trifonova et al., Antimicrob. Agents Chemother. 50:4005, 2006; Fichorova et al., mBio 2:e00168, 2011, each of which is incorporated herein by reference in its entirety). Release of markers of cell damage (e.g., increase in CD4, IL-1β, CXCL8, CCL2, CCL21, EMP1; decrease in BPI) and production of inflammatory mediators, such as IL-1, IL-8, TLR4, may be used as markers of irritation and pro-inflammation (see, also, Fichorova et al., Toxicol. Appl. Pharmacol. 285:198, 2015; Doncel et al., J. Acquir. Immune Defic. Syndr. 37(Suppl. 3):S174, 2004; Fichorova et al., Biol. Reprod. 71:761, 2004; Moench et al., BMC Infect. Dis. 10:331, 2010, each of which is incorporated herein by reference in its entirety). Biomarkers of epithelial integrity and immune function have been validated in multiple clinical studies of vaginal product safety (Mauck et al., AIDS Res. Hum. Retroviruses 29:1475, 2013; Fichorova et al., mBio, 6: e00221, 2015; Fichorova et al., Cytokine 55:134, 2011; Mauck et al., J. Acquir. Immune Defic. Syndr. 49:243, 2008; Morrison et al., J. Acquir. Immune Defic. Syndr. 66:109, 2014; Schwartz et al., Contraception 74:133, 2006; Keller et al., J. Antimicr. Chemother. 51:1099, 2003, each of which is incorporated herein by reference in its entirety). A composition of the present disclosure may be considered non-irritating and non-inflammatory if it does not cause more than a 25% release of markers of cell damage or expression of pro-inflammatory mediators above that caused by a negative control (e.g., synthetic TLR2/6 ligand). In some embodiments, the compositions of the present disclosure may be considered non-irritating and non-inflammatory to genital skin and/or mucosa if application of the composition to the specific region does not increase inflammasome and/or cytokine production (e.g., by more than 1%, more than 5%, more than 10%, more than 15%, more than 20%, more than 25%) as compared to control. For example, a control reference level may be the level of the indicated biomarker expressed as an average of the level of the biomarker from samples taken from a control population of healthy subjects. In some embodiments, a control reference level may be the level of the indicated biomarker expressed as an average of the level of the biomarker measured from a subject given a control formulation. Suitable samples or references for determining reference levels include healthy cells. In some embodiments, the reference to determine the reference level of an indicated biomarker may be a derived from the subject, a healthy subject, or a population of subjects.

Healthy mucin and mucus, and mucin-regulating enzyme production (glycosidases), from the vaginal epithelial cells can be determined following exposure of genital tissue or fluid to compositions used for cleaning and lubricating the anogenital region. Current urogenital and/or anogenital products can damage the natural mucus protection barrier of the surface of genital skin or mucosa of the vagina, penis and urethra, through altering mucin production and enhancing mucin-degradation. In particular, mucin degradation can occur following exposure to certain pathogenic bacteria or to certain ingredients (e.g. carbomer and oils) commonly found in products used in the genital region. Mucin quality from the vagina can be determined following exposure to a composition of the present disclosure by testing CVF samples collected using a SoftCup or similar menstrual cup device covering the base of the cervix and performing ELISA assay (enzyme linked immunosorbent assay) to measure mucins and ELLA assay (enzyme linked lectin assay) to measure carbohydrate structures as described in Moncla et al. (PLOS One 11:e0158687, 2016, incorporated herein by reference in its entirety). Glycosidase assays can be performed to measure enzyme specific activity as described in Moncla, incorporated herein by reference in its entirety). A composition of the present disclosure is considered non-harmful to genital mucin and mucus if it does not inhibit mucus viscosity or production (e.g., by more than 1%, 5%, 10%, 15%, 20%, 25%, 30%, or 35%), or increase production of glycosidases (e.g., by more than 1%, 5%, 10%, 15%, 20%, 25%, 30%, or 35%) (see, e.g., Moncla). Compositions of the present disclosure may also be tested for their effects in vaginal infection susceptibility models, such as a mouse genital herpes transmission model (see, e.g., Moench et al., BMC Infect. Dis. 10:331, 2010). Increased susceptibility to infection transmission may be caused by damage to vaginal epithelial cells.

Effects of topical compositions on tissue viability using tissue models (e.g. human explants or cell monolayers) may also be assessed using the MTT colorimetric assay technique. The MTT assay is a colorimetric assay for assessing cell metabolic activity. NAD(P)H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to its insoluble formazan, which has a purple color. The MTT assay may be used to measure a composition's cytotoxicity or effect on cell viability (see, e.g., Ayehunie et al., 2011).

In addition, oxidative stress and antioxidant potential of the tissues can be determined by common methods, such as a TBARS assay to evaluate the impact of various embodiments on tissue health. Because reactive oxygen species (ROS) have extremely short half-lives, they are difficult to measure directly. Instead, several products of the damage produced by oxidative stress, such as thiobarbituric acid reactive substances (TBARS), can be measured. TBARS are formed as a byproduct of lipid peroxidation (i.e., as degradation products of fat), which can be detected by the TBARS assay using thiobarbituric acid as a reagent.

The in vivo rabbit vaginal irritation (RVI) model may also be used to assess the irritation and inflammatory characteristics of a formulation (see, e.g., Eckstein et al., J. Reprod. Fertil. 20:85, 1969). This model is based on macroscopic observations of erythema, edema and ulceration and histopathologic analysis of the tissues collected after exposure of the animals to the test materials. A non-irritating and safe composition of this disclosure would show no negative macroscopic or histopathologic effects as compared to a control vehicle. An expanded RVI model having a quantitative nuclease protection assay (qNPA) to quantify mRNA levels of 25 genes representing leukocyte differentiation markers, toll-like receptors (TLR), cytokines, chemokines, epithelial repair, microbicidal and vascular markers has also been described (see, e.g., Fichorova et al., Toxicol. Appl. Pharmacol. 285:198, 2015).

Sensitization tests may be used evaluate the potential of a composition of the present disclosure to cause a sensitizing effect or allergenic reaction in a subject over an extended period of exposure. Exemplary tests include Guinea pig tests, such as the Magnusson-Kligman guinea pig maximization test (J. Invest. Dermatol. 52:268, 1969), the occluded patch test of Buehler (Arch. Dermatol. 91:171, 1965), and the open epicutaneous test (see, e.g., Kero et al., Contact Dermatitis 6:341, 1980). A murine local lymph node assay (LLNA) is another method for the identification of skin sensitizing chemicals. In contrast to guinea pig tests, this assay relies on measurement of events induced during the induction phase of skin sensitization, specifically lymphocyte proliferation in the draining lymph nodes which is a hallmark of a skin sensitization response, rather than the elicitation phase (see, e.g., Kimber et al., Contact Dermatitis 21:215, 1989; Basketter et al., Food Chem. Toxicol. 34: 985, 1996). The human repeat-insult patch test (HRIPT) may be performed as a confirmatory test in the safety evaluation of skin sensitizers. Sensitization is a process by which humans develop increased allergic responses to a substance over time through repeated exposure to that substance. It is different from irritation because it involves an immune response. Skin sensitization reactions are usually characterized by erythema coupled with one or more of various dermal sequelae, such as edema, papules, vesicles, bullae, and/or pruritus (see, e.g., McNamee et al., Regul. Toxicol. Pharmacol. 52:24, 2008).

In yet another example, in vivo colposcopy exams of women following use of compositions of the present disclosure can identify signs of inflammation or irritation. User surveys can also be used for scoring of symptoms of the same (see, e.g., Van Damme et al., Lancet 360:971, 2002; Bunge et al., J. Acquir. Immune Defic. Syndr. 60:337, 2012).

Irritating topical products may trigger the release of pro-inflammatory cytokines (e.g., TLR, IL-1, IL-6, IL-8, TNF-α, IFN-γ, IL-17) and inflammasomes (e.g., NLRP3 and NLRC4). Cytokines and inflammasomes can be measured using an enzyme-linked immunosorbent assay (ELISA), quantitative PCR, or other molecular assay. A product is considered non-inflammatory if it does not cause increased expression of pro-inflammatory cytokines or inflammasomes (Rabeony et al., Eur. J. Immunol. 45:2847, 2015).

In certain embodiments, the compositions of the present disclosure do not induce irritation or inflammation potential in the anogenital region subject greater than 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% as compared to an untreated control subject, preferably as measured using the slug mucosal irritation test of Adriaens et al. (2008).

(3) Evaluating Effect on Gametes

In certain embodiments, the compositions of the present disclosure do not impact sperm viability or function. In certain embodiments, compositions of the present disclosure are designed to mimic the vaginal environment during a woman's fertile window around ovulation and not negatively impact sperm viability or function. In some embodiments, the compositions of the present disclosure are designed to mimic the vaginal environment during a woman's non-fertile period and not impact sperm viability or function. Assays or models for assessing sperm survival and function, include for example, sperm motility assays (e.g., subjective or computer assisted), sperm viability studies, in vitro fertilization and embryo development animal models, membrane integrity of sperm, survival time in culture, cervical mucus penetration, lipid peroxidation, capacitation, zona recognition, acrosome reaction and sperm-oocyte fusion, and sperm chromatin testing (reviewed in, e.g., Vasan, Indian J. Urol. 27:41, 2011; Oehninger et al., Fertil. Steril. 102:1528, 2014; Mortimer et al., Hum. Reprod. Update 19 (Suppl 1):i1-i45; 2013, each of which is incorporated by reference in its entirety). Additional testing can include post-coital testing to evaluate sperm presence in the cervical canal, and even pregnancy outcomes in an animal model or among women in a clinical trial.

Sperm motility is one function that may be used to assess sperm function and thus fertilization potential. Motility of sperm is expressed as the total percent of motile sperm, the total percent of progressively motile sperm (swimming forward), or the speed of sperm that are progressively motile. These measurements may be made by a variety of assays. For example, either a subjective visual determination is made using a phase contrast microscope when the sperm are placed in a hemocytometer or on a microscope slide, or a computer assisted semen analyzer is used. Under phase contrast microscopy, motile and total sperm counts are made, and speed is assessed as fast, medium or slow. A more specific measurement of sperm motility is motility grade, where the motility of sperm is divided into four different grades (Cooper et al., Human Reprod. Update 16:231, 2010, incorporated by reference in its entirety). Grade A refers to sperm with progressive motility that are the strongest and swim fastest in a straight line. Grade B refers to sperm with non-linear motility; that move forward but tend to travel in a more curved or crooked motion. Grade C sperm have non-progressive motility in that they do not move forward despite tail movement. Grade D sperm are immotile. Using a computer assisted semen analyzer (such as IVOS Hamilton Thorne, Beverly, Mass.), the motility characteristics of individual sperm cells in a sample are objectively determined (Hum. Reprod. 13:142, 1998). Briefly, a sperm sample is placed onto a slide or chamber designed for the analyzer. The analyzer tracks individual sperm cells and determines motility and velocity of the sperm. Data may be expressed as percent of total motility, and measurements are obtained for path velocity and track speed as well. It is known that the velocity of sperm is often impacted by the viscosity of a medium and separate from the toxicology of that medium as described in J Elgeti, et al., Biophys J 99 (2010): 1018-1026, hereby incorporated by reference in its entirety.

The Human sperm survival assay is typically used in human in vitro fertilization (IVF) programs as described in C De Jonge et al., J Androl 24 (2003): 16-18, and A Hossain, et al., Adv Urol 2010 (2010): 136898, each of which is hereby incorporated by reference in its entirety. It has also been proposed as a sensitive cytotoxic assay for any topical products, as sperm show damage before other cell monolayer types. The test requires sperm suspension culture with 10% product for 24 hours and evaluation of the percent motile, progressive motility and/or total motility of sperm at the end of culture in climate-controlled settings. The test can also be replicated with bull sperm for controlling for individual sperm sample effect. In certain embodiments, the compositions of the present disclosure are considered not toxic to sperm if application to a subject does not cause a decrease in sperm survival greater than 0.5%, greater than 1%, greater than 2%, greater than 3%, greater than 4%, greater than 5%, greater than 6%, greater than 7%, greater than 8%, greater than 9%, greater than 10%, greater than 11%, greater than 12%, greater than 13%, greater than 14%, greater than 15%, or greater than 20% as compared to control.

Sperm viability can be measured using several different methods. By way of example, two of these methods are staining with membrane exclusion stains and measurement of ATP levels. Briefly, a sample of sperm is incubated with a viable dye, such as Hoechst 33258 or eosin-nigrosin stain. Cells are placed in a hemocytometer and examined microscopically. Dead sperm with disrupted membranes stain with these dyes. The number of cells that are unstained is divided by the total number of cells counted to give the percent live cells. ATP levels in a sperm sample are measured by lysing the sperm and incubating the lysate with luciferase, an enzyme obtained from fireflies, which fluoresces in the presence of ATP. The fluorescence is measured in a luminometer. The amount of fluorescence in the sample is compared to the amount of fluorescence in a standard curve allowing a determination of the number of live sperm present in the sample.

Membrane integrity of sperm may be assayed by a hypo-osmotic swell test which measures the ability of sperm to pump water or salts if exposed to non-isotonic environments. Briefly, in the hypo-osmotic swell test, sperm are suspended in a solution of 75 mM fructose and 25 mM sodium citrate, which is a hypo-osmotic (150 mOsm) solution. Sperm with intact, healthy membranes pump salt out of the cell causing the membranes to shrink as the cell grows smaller. The sperm tail curls inside this tighter membrane. Thus, sperm with curled tail are counted as live, healthy sperm with normal membranes. When compared to the total number of sperm present, a percent of functional sperm may be established.

The degree of membrane integrity may be determined by lipid peroxidation (LPO) measurements, which assess sperm membrane damage generated by free radicals released during handling. Lipid membrane peroxidation is assayed by incubating sperm with ferrous sulfate and ascorbic acid for one hour in a 37° C. water bath. Proteins are precipitated with ice-cold trichloroacetic acid. The supernatant is collected by centrifugation and reacted by boiling with thiobarbituric acid and NaOH. The resultant malondialdehyde (MDA) formation is quantified by measuring absorbance at 534 nm as compared to an MDA standard (Bell et al., J. Andrology 14:472, 1993, incorporated herein by reference in its entirety). LPO is expressed as nM MDA/10⁸ sperm. A composition of the present disclosure has a stabilizing effect on sperm if exposure results in decreased LPO production.

The stability of chromatin DNA is assayed using the sperm chromatin structure assay (SCSA). Sperm cells are very sensitive to oxidative stress resulting in sperm chromatin (DNA) damage (Whittington et al., Int. J. Andrology 22:236, 1999; Pasqualotto et al., Fertility and Sterility 73:459, 2000; Kodama et al., Fertility and Sterility 68:519, 1997, each of which is incorporated herein by reference in its entirety). This damage can be profound in sperm cells because they contain little to no mechanisms to repair DNA damage after it occurs. The sperm chromatin assay is based on the metachromatic staining of single and double stranded DNA by acridine orange stain (Evenson et al., Human Reprod. 14:1039, 1999; Evenson et al., J. Andrology 23:25, 2002, each of which is incorporated by reference in its entirety). Excitation with an argon laser causes acridine orange intercalated in double-stranded DNA to emit a green fluorescence, whereas red fluorescence is emitted by single strand DNA. The extent of DNA denaturation in a sample is expressed as a and calculated by the formula α=red/(red+green). The endpoint measurement is DNA Fragmentation Index (DFI). A DFI of <15% DFI indicates excellent to good sperm DNA integrity. Fresh sperm samples are incubated for a period of time in the presence of a test composition, flash frozen, and subsequently assayed for DNA breakage (see, e.g., Evenson et al., J. Androl. 23:25, 2002, incorporated herein by reference in its entirety). Other DNA assays for the stability of chromatin DNA include the terminal deoxynucleotidyl transferase-mediated fluorescein-dUTP nick end labelling (TUNEL) test; COMET assay and Sperm Chromatin Dispersion as disclosed in D Evenson, Anim Reprod Sci 169 (2016): 56-75, hereby incorporated by reference in its entirety.

In vitro fertilization rates are determined by measuring the percent fertilization of oocytes in vitro in an animal model such as bovine or murine model. For example, maturing bovine oocytes are cultured in vitro in M199 medium plus 7.5% fetal calf serum and 50 μg/ml luteinizing hormone for 22 hours. Following culture for 4 hours, the sperm are chemically capacitated by adding 10 IU of heparin and incubated with bovine oocytes for 24 hours. At the end of the incubation, oocytes are stained with an aceto-orcein stain or equivalent to determine the percent oocytes fertilized. Alternatively, fertilized oocytes may be left in culture for 2 days, during which division occurs and the number of cleaving embryos (i.e., 2 or more cells) is counted.

Survival time in culture of sperm (time to loss of motility) is another convenient method of establishing sperm function. Briefly, an aliquot of sperm is placed in culture medium, such as Tyrode's medium, pH 7.4 and incubated at 37° C., 5% CO₂, in a humidified atmosphere. At timed intervals, for example every 2 hours, the percentage of motile sperm in the culture is determined by visual analysis using an inverted microscope or with a computer assisted sperm analyzer. As an endpoint, a sperm sample is considered no longer viable when less than 5% of the cells have progressive motility.

Another parameter of sperm function is the ability to of sperm to swim up into a column of cervical mucus or substitute (reviewed in Ola et al., Hum. Reprod. 18:1037-1046, 2003, incorporated by reference in its entirety). This cervical mucus penetration test can be done either in vitro or in vivo. Sperm are placed at one end of the track and the distance that sperm have penetrated into the mucus after a given time period is determined. Cervical mucus penetration studies offer valuable biocompatibility data for devices that are used for reproductive purposes. The bovine cervical mucus penetration study is an excellent in vitro assay to evaluate sperm penetration into cervical mucus. Bergman et al. (Fertility and Sterility 36:363-367, 1981, incorporated herein by reference in its entirety) found excellent correlation between sperm penetration into frozen bovine cervical mucus and fresh human cervical mucus (r=0.98) due to the similarity of human and bovine cervical mucus rheological and biophysical make-up (Bergman et al., Fertil. Steril. 36:363-367, 1981; Keel et al., Arch. Androl. 44:109-115, 2000, each of which is incorporated herein by reference in its entirety). These assays may be used to evaluate cytotoxicity of aspects of the present disclosure, such as applicators, compositions, and kits, by incubating the embodiment (e.g. applicator) in a container with a mixture of the composition (e.g. 10% composition by volume of the solution), cervical mucus and a sperm solution, in order to determine toxicity of leached products from the device on sperm penetration into cervical mucus. Toxicity of sperm penetration can also be measured by placing the compositions of the present disclosure in an applicator (e.g., an applicator as described herein), and incubating the composition with the applicator for a set time (e.g. more than 10 min, more than 20 min, more than 25 min, 30 min), where after the incubated composition is mixed with cervical mucus and sperm to form a solution. This solution may be used to evaluate subsequent sperm penetration into mucus, to determine effects of sperm exposed to the composition and/or identify the presence of any leached chemicals from the applicator into the composition. In certain embodiments, the compositions of the present disclosure are not toxic to sperm if there is not a decrease in sperm penetration of greater than 0.5%, of greater than 1%, of greater than 2%, of greater than 3%, of greater than 4%, of greater than 5%, of greater than 6%, of greater than 7%, of greater than 8%, of greater than 9%, of greater than 10%, of greater than 11%, of greater than 12%, of greater than 13%, of greater than 14%, of greater than 15% of control.

Other assays for sperm function include global proteomic or metabolomic assays, or assays providing metatranscriptomic (mRNA), or glycomic (sugars in situ) analysis of sperm (e.g., 24 hr following ejaculation). These assays may evaluate sub-cellular changes for proteins related to hyperactivation, capacitation, acrosome reaction and zona-pellucida binding (fertilization process proteins). Such assays may be conducted using liquid chromatography/mass spectroscopy (LC-MS-MS) or alternatively NMR and data matched to establish proteomics online databases.

In other aspects, microbiome (e.g., penile, genital) sequencing from a subject with V4 16s rRNA gene copies or whole genome sequencing may be performed. Microbial DNA may be extracted from swabs taken of the microbiome region to be measured using standard extraction protocols and sequenced on high through put genomic sequencing platforms such as nanopore sequencing, or next generation sequencing platforms (such as Illumina Hi seq). Using online bioinformatics pipelines standard to the industry, raw reads from the sequencing data may be curated and filtered for quality control. Finalized sequenced data can be compared to annotated reference databases in order to determine the composition of a sample's microbiome, and biological observation matrices may be employed, for example, for visualization and statistical analyses. Furthermore, correlation analysis of microbiota in the penile region or semen may be used to determine if a sample needs optimization such as being associated with known pathogenic bacteria. The status of a sample can be compared to other sperm function tests to determine if there are any pathogenic bacteria, and in particular, pathogenic bacteria that may be causing sperm infertility issues.

Alternatively, sperm penetration of mucus may be measured in vivo in women. At various times post-coitus, a sample of cervical mucus is removed and examined microscopically for the number of sperm present in the sample. In the post-coital test, improved sperm function is established if more sperm with faster velocity are seen in the mucus sample after exposure to a composition of the present disclosure versus a sample of mucus from the patient after exposure to a control lubricant.

Other assays of sperm function potential include the sperm penetration and hemizona assays. In the sperm penetration assay, the ability of sperm to penetrate into an oocyte is measured. Briefly, commercially available zona free hamster oocytes are used (EmbryoTech Laboratories, Haverhill, Mass.). Hamster oocytes are suitable in this assay for sperm of any species. Capacitated sperm, such as those cultured with bovine serum albumin for 18 hours, are incubated for 3 hours with the hamster oocytes. Following incubation, oocytes are stained with acetolacmoid or equivalent stain and the number of sperm penetrating each oocyte is counted microscopically. A hemizona assay measures the ability of sperm to undergo capacitation and bind to an oocyte. Briefly, in this assay, live normal sperm are incubated in media with bovine serum albumin, which triggers capacitation. Sperm are then incubated with dead oocytes which are surrounded by the zona pellucida, an acellular coating of oocytes. Capacitated sperm bind to the zona and the number of sperm binding is counted microscopically.

In certain embodiments, a composition of the present disclosure may be considered non-toxic to sperm if following exposure to a 10% solution of the composition, sperm retain at least 80%, at least 85%, at least 90%, or at least 95% motility as compared to sperm exposed to a control medium.

In certain embodiments, the topical, isotonic composition of the present disclosure: (i) is non-irritating to the urogenital and/or anogenital mucosa or skin; (ii) does not promote growth of pathogenic bacteria of the urogenital and/or anogenital region; (iii) does not reduce the healthy microbiota of the genital region more than 25%; (iv) does not disrupt or reduce mucin production by the urogenital and/or anogenital mucosa or skin; (v) does not cause more than a 20% increase in inflammation of the urogenital and/or anogenital mucosa and/or the skin genital tissue; (vi) does not disrupt genital fluid function; (vii) is non-toxic to sperm; (viv) has a prebiotic effect on Lactobacillus species growth found in the genital tissues; (viv) decreases vaginal, bacterial, fungal, or viral infection rates by 5% or more; or any combination thereof.

As discussed in HEIM Janneke et al., (2019) doi: 10.1111/1471-0528.15870, hereby incorporated by reference in its entirety, vaginal probiotics often involve the direct addition of Lactobacillus strains to the urogenital and/or anogenital region, but have significant drawbacks including regulatory barriers. The present disclosure is partially premised on compositions which are primarily focused on creating the optimal microbiotic environment for healthy growth (e.g. Lactobacillus growth) in the anogenital and/or urogenital microbiomes. In some embodiments, the compositions may be used for the treatment or prophylaxis of a disease, disorder, or condition associated with dysbiosis of the urogenital and/or anogenital region in a subject in need thereof. The method for the treatment of the treatment or prophylaxis of a disease, disorder, or condition associated with dysbiosis of the urogenital and/or anogenital region in a subject in need thereof may comprise administration of a composition of the present disclosure (e.g., a topical isotonic composition comprising bornyl acetate, a prebiotic oligosaccharide, and a metal co-factor) to the urogenital and/or anogenital region of the subject. In some embodiments, the urogenital and/or anogenital region is the vagina. In some embodiments, the composition is administered topically to the vagina. The method may further comprise measuring the pH of the vagina (e.g., with a pH nanosensor or other method) prior to application, and administering a composition to affect the pH of the microbiome environment in order to physiologically optimize Lactobacillus dominance. In various embodiments, the disease, disorder, or condition associated with dysbiosis of the urogenital and/or anogenital region may be selected from diabetes, lichen sclerosus, urinary incontinence, provoked vestibulodynia, vulvodynia, genital syndrome of menopause, interstitial cystitis, autoimmune genital disease, dyspareunia, or infertility. In several embodiments, the compositions may decrease vaginal infection rates and/or urinary tract infections. In various embodiments, the disease, disorder or condition associated with dysbiosis of the urogenital and/or anogenital region may be selected from sexually transmitted diseases (e.g., HPV, HSV, HIV), cervical cancer, pelvic floor disorder, genitourinary syndrome of menopause, provoked vestibulodynia, vulvodynia, interstitial cystitis, autoimmune genital disease, dyspareunia, BV or infertility. In several embodiments, the compositions may decrease vaginal infection rates and/or urinary tract infections. In several embodiments, the compositions decrease persistence of high-risk HPV. Similarly, the anal microbiome has certain tolerances wherein the compositions can be used to promote healthy growth therein. Measurements of the surface pH and human rectal mucosa has been measured to have a pH of from 6.26 to 6.98, as shown in N McNeil, Gut 28 (1987): 707-713, hereby incorporated by reference in its entirety. The rectum has been shown to have a more alkaline pH of 7.9 as shown in W Bitterman, et al., Gastroenterology 53 (1967): 288-290, hereby incorporated by reference in its entirety. In some embodiments, compositions for the treatment of the anal microbiome (e.g., lubricants) may have a pH of from pH 5.5 to pH 8 (e.g., from pH 5.5 to pH 7, from pH 5.8 and pH 6.2).

In yet another aspect, the present disclosure provides a method for collecting the genital microbiome from a donor dyad member comprising topically administering an effective amount of a topical, isotonic composition of the present disclosure to the urogenital and/or anogenital region of the donor dyad member and collecting the topical, isotonic composition from the urogenital and/or anogenital region of the subject. In certain embodiments, the topical, isotonic composition is collected in a receptacle. In certain embodiments, the topical, isotonic composition is administered via a wipe, adhesive roller, a blanket, an undergarment, diaper, film, or aerosol. In some embodiments, the composition is integrated into a tampon, vaginal ring, cervical cup, diaphragm, or condom, wherein the composition will be released upon insertion into the urogenital and/or anogenital region.

In certain embodiments, the collected topical, isotonic composition is assayed for the presence of pathogenic microorganisms; cultured to identify and propagate beneficial microorganisms (e.g., Lactobacilli); or both. In certain embodiments, the beneficial microorganisms are isolated and added as a probiotic or by vaginal flora transfer to a separate, topical, isotonic composition of the present disclosure for administration to a recipient dyad member or other unrelated individual. In certain embodiments, the donor dyad member and recipient dyad member are members of a sexual dyad, e.g., a heterosexual dyad, homosexual dyad, or other sexual orientation dyad.

The individual components of the compositions described herein (e.g., prebiotic oligosaccharides, metal co-factors, bornyl acetate, essential oils comprising bornyl acetate) may be used for application of a subject in need thereof. In some embodiments, these individual components (e.g., prebiotic oligosaccharides, metal co-factors, bornyl acetate, essential oils comprising bornyl acetate) may be used for the preparation of a medicament (e.g., topical compositions, isotonic compositions, topical isotonic compositions) for the treatment of a subject in need thereof. For example, the individual components or the medicament may be administered to the subject in order to hydrate the urogenital and/or anogenital region of the subject and/or lubricate the anogenital region of the subject and/or clean the urogenital and/or anogenital region of the subject and/or decrease irritation or inflammation of the urogenital and/or anogenital region of the subject and/or enhance the genital microbiota of the subject. In certain embodiments, the individual components and compositions described herein may be used for the treatment or the prophylaxis of the dysbiosis of a subject in need thereof

Applicators

The present disclosure also includes applicators which may be used for administration to the urogenital and/or anogenital region of a subject (e.g., the vagina, the penis). The applicator may comprise a storage portion having an internal reservoir capable of storing one or more doses of the compositions of the present disclosure. In some embodiments, the internal reservoir may have a volume of from 1 mL to 60 mL or 1 mL to 5 mL or 5 mL to 60 mL or 10 mL to 50 mL or 15 mL to 30 mL or 20 mL to 25 mL. The internal reservoir may be in fluid communication with an application portion configured to release an amount of the composition. For example, a user may apply to a force to the storage portion causing the composition to be expelled therethrough.

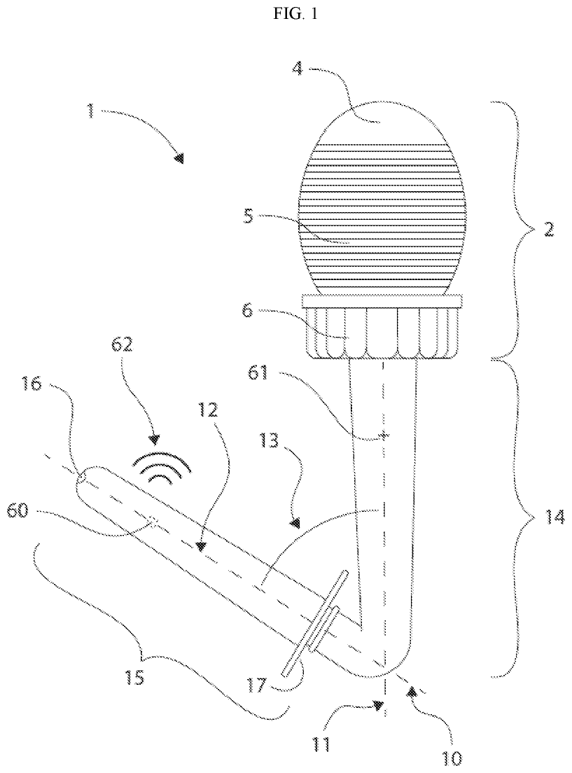

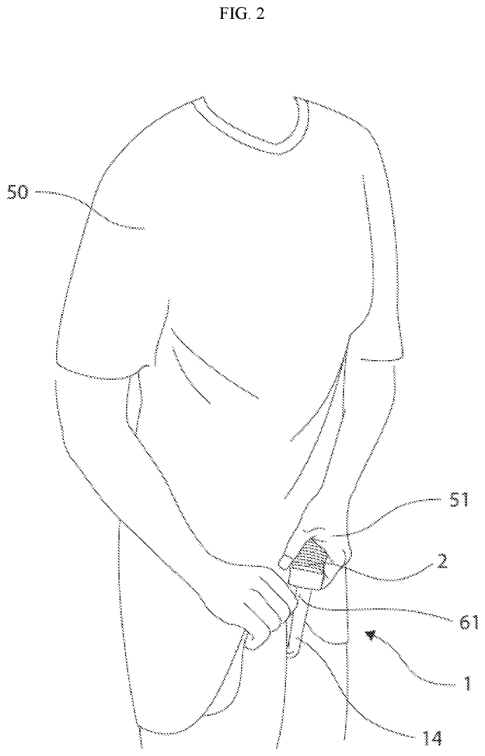

The present disclosure also includes applicators which may be used for administration to the urogenital and/or anogenital region of a subject (e.g., the vagina). As shown in FIG. 1, applicator 1 comprises storage portion 2 and delivery portions 14 and 15. Storage portion 2 comprises an internal reservoir configured to house a composition (e.g., a composition as disclosed herein for application to the urogenital and/or anogenital region). Storage portion 2 comprises bulb 4 with gripping elements 5 and connector 6 which connects storage portion 2 to delivery portion 14. The connector portion may be configured to connect to delivery portion 14 through one or more removable connectors such as threading on both elements, snap connections, and the like. When connected, the internal reservoir is in fluid communication with an internal flow element in delivery portions 14 and 15. Force applied to bulb 4 from a user causes composition within the internal reservoir to flow through delivery portions 14 and 15 and out exit orifice 16. Delivery portion 14 has a central axis 11 and delivery portion 15 has a central axis 10. These two axes are angled with respect to one another at an angle 13 of, for example, less than about 90° or less than about 60° or less than about 45° or less than 30° or less than about 20°. In some embodiments, the angle between axis 10 and axis 11 is movable such that it may be set by a user. Delivery element 15 is dimensioned for insertion into the urogenital and/or anogenital region of a user. For example, delivery element 15 may be substantially cylindrical about its major axis 10 and comprises a rounded distal end at exit orifice 16. In some embodiments, delivery element 15 comprises a stopper 17 which indicates to a user the maximum depth of insertion for the device. In the embodiment depicted, stopper 17 comprises two annular rings surrounding around delivery element 15 to prevent further insertion of the applicator. In some embodiments, the length of delivery element (i.e., the length along axis 10) between exit orifice 16 and stopper 17 is less than about 10 cm or less than about 9 cm or less than about 8 cm or less than about 7 cm or less than about 6 cm or less than about 5 cm. In some embodiments, the maximum circumference of delivery element 15 is less than about 8 cm or about 7 cm or about 6 cm or less than about 5 cm or less than about 4 cm or less than about 3 cm. FIG. 2 illustrates the use of applicator 1. A user 50 holds storage portion 2 in their hands 51. Due to the multiple axes of in the delivery portions, delivery portion 15 may be easily inserted for application of the composition to the user, while delivery portion 14 is not. As can be seen, application of composition stored within storage portion 15 to the urogenital and/or anogenital region may occur with one handed insertion into the urogenital and/or anogenital region without having to remove clothes or spread patient legs. The internal reservoir may hold an amount of composition for a single use or have multiple uses. In some embodiments, the internal reservoir may have a volume of from about 5 mL to about 60 mL or about 10 mL to about 50 mL or about 15 mL to about 30 mL or about 20 mL to about 25 mL.

The applicator may further comprise a sensor capable of measuring one or more characteristics of the surrounding environment (e.g., the penis, the vagina) including the pH. For example, the applicator may comprise a litmus or nitrazine dye which, following insertion, is capable if visually displaying pH information the environment of the urogenital and/or anogenital regions. In some embodiments, the applicator may comprise a nanosensor such as that disclosed in U.S. Pat. No. 10,436,745, hereby incorporated by reference in its entirety and particularly in relation to pH nanosensors. In some embodiments, the nanosensor is capable of measuring the pH of the surrounding environment (e.g., the anogenital and/or urogenital region). Following measurement, the nanosensor may be capable of communicating the pH measurement (e.g., with Bluetooth, radio frequency identification, USB such as USB-A, USB-B, USB-C, micro USB, lightning cable) with an external device such as a laptop, tablet, computer, server, and/or smart phone. The nanosensor may transmit the value of the measured pH or information relating to the pH. For example, the sensor may transmit a binary signal and/or a ternary signal depending on user settings. The external device may be configured to interpret, display, and track such measurements.

Another embodiment may provide an applicator comprising a nanosensor, such as a solid-state sensor based on oxidized single-walled carbon nanotubes (ox-SWNTs) functionalized with a conductive polymer (e.g., poly(l-aminoanthracene) (PAA)). The nanosensor may, in some embodiments, have a Nernstian response over a wide pH range (e.g., pH 2-pH 12) and retain sensitivity over 120 days. Another embodiment may provide for an applicator comprising a nanosensor attached to a passively-powered radio-frequency identification (RFID) tag, which may transmit information (e.g., pH data) to a mobile or portable device (e.g., tablet, smart phone) accessible through a software application or to a computer having the software application. This battery-less, reference electrode-free, wirelessly transmitting sensor platform may be used for biomedical applications, including but not limited to, intravaginal pH measurement or penile pH measurement, such as when attached to an applicator or probe. In some embodiments, the sensor platform includes a system for communicating with an external device such a blue-tooth communication system, a physical connection to an external device such as USB or lightning cable. In some embodiments, power is delivered to the sensing medium through the physical connection to the external device.

In some embodiments, the applicator may have a display 61 operable connected to the nanosensor. The display 61 may be configured to illustrating a binary signal such as a “+” symbol indicating that the pH is above a value such as 5.5. In some embodiments, the display 61 be configured with a ternary signal set up indicating whether the pH is below a range (e.g., below 4) within a range (e.g., from 4-5) or above a range (e.g., above 5). In some embodiments, the display 61 may show the pH measured from the nanosensor. In various embodiments, the sensor may show a binary display symbols (e.g., a “+” for a pH measurement below a certain level such as pH 4.8 and a “−” for a pH measurement above and/or equal to that level such as pH 4.8). In some embodiments, the display 61 may be configured to display ternary display symbols (e.g., a “0” if the measured pH is at a pH or within a certain pH range, a “−” if the pH is below that certain pH or pH range, and a “+” if the measured pH is above that certain pH or pH ranges). In some embodiments, the display 61 may be configured to display ternary symbols wherein the certain pH range may span from pH 4.25 to pH 4.75.

In other embodiments, the applicator described herein may have a nanosensor configured to communicate with an application, where the application may track and monitor vaginal pH using, for example, a carbon-fiber nanosensor input and may report one or more metabolomic characteristics of the biological sample such as pH, the presence of symptoms and/or severity of the disease or condition in connection with microbiome ecosystem, or combinations thereof. The application may allow for vaginal pH monitoring by storing pH measurement history and showing pH measurement changes locally on a selected digital device (e.g., a computer, laptop, tablet, mobile device, smart phone, server), or making such information available for sharing or downloading, or a combination thereof.

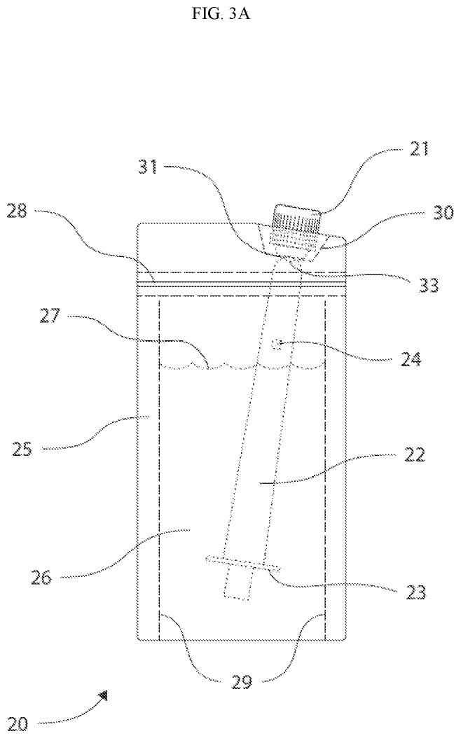

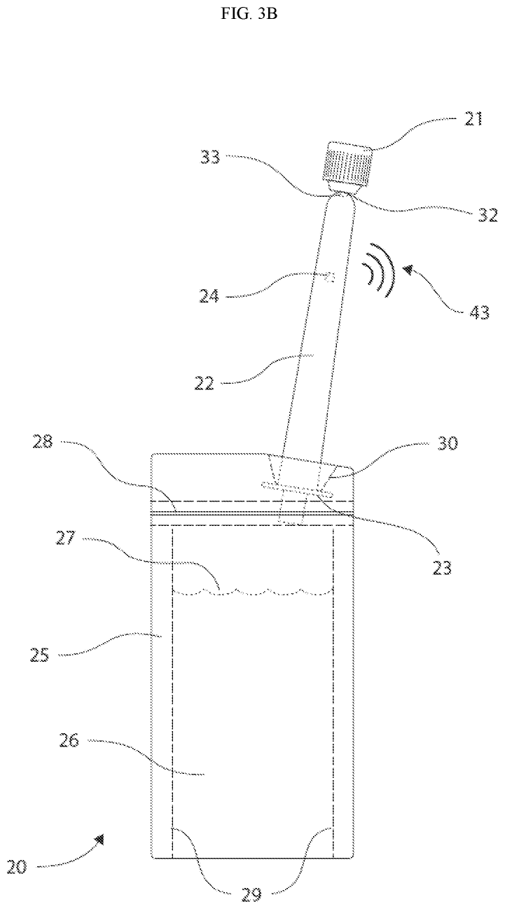

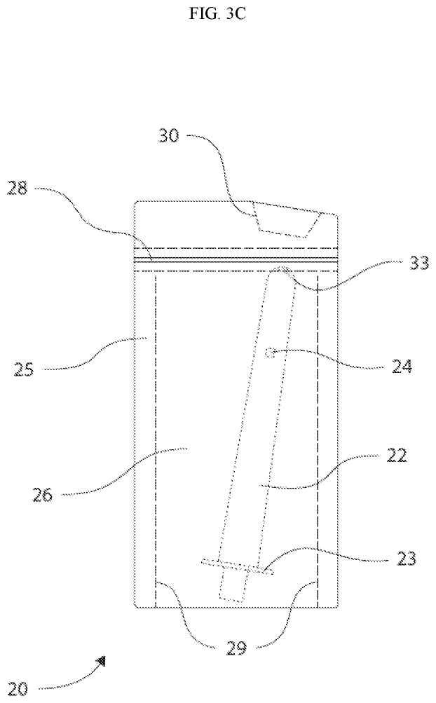

Another applicator is illustrated in FIGS. 3A-C. Applicator 20 is illustrated in storage (FIG. 3A), nearly application (FIG. 3B), and disposal (FIG. 3C) configurations. Applicator 20 comprises a container 25 such as a bag or pouch comprising an internal reservoir 26 as defined by seams 29. The pouch is removably sealed with closure 28. The applicator 20 may comprise a composition 27 for application such as, for example, a composition as disclosed herein. Applicator portion 22 (also referred to herein as a delivery element) comprises a stopper 23, and an internal flow chamber extending from a distal end of applicator portion 22 to exit orifice 33. Applicator portion 22 may comprise a nanosensor 24. In some embodiments, the applicator portion may comprise a measurement device capable of transmitting pH information of an environment (e.g., litmus paper). In the storage configuration (FIG. 3A), applicator portion 22 is contained within the internal reservoir. A twistable cap is connected to a cut out 30 on container 25 allowing for sealing of the applicator during storage at interface 31. In the embodiment depicted, the cap 21 is disposed proximal to exit orifice 33 of applicator portion 22. The applicator portion may be forcibly removed from the container 25 by, for example, supply a force to cap 21 in a direction away from internal reservoir 26. Such force may break the interface 31 between cap 21 and cutout 30 allowing the applicator to be removed from internal reservoir 26. In some embodiments, the length applicator portion 22 (i.e., the length along the major axis) between exit orifice 33 and stopper 23 is less than about 10 cm or less than about 9 cm or less than about 8 cm or less than about 7 cm or less than about 6 cm or less than about 5 cm. In some embodiments, the maximum circumference of applicator portion 22 is less than about 8 cm or about 7 cm or about 6 cm or less than about 5 cm or less than about 4 cm or less than about 3 cm. The internal reservoir may hold an amount of composition for a single use or have multiple uses. In some embodiments, the internal reservoir may have a volume of from about 5 mL to about 60 mL or about 10 mL to about 50 mL or about 15 mL to about 30 mL or about 20 mL to about 25 mL.

As can be seen in FIG. 3B, the applicator may be pulled out of the container where the stopper 23 and cutout 30 are adapted to prevents complete removal of applicator portion 22 and keeping exit orifice 32 in fluid communication with internal reservoir 26. FIG. 3B illustrates applicator 20 with applicator portion 22 removed from internal reservoir 26. Once in this configuration, applicator 20 may be placed in the application configuration by removal of cap 21, for example, by twisting the cap to expose exit orifice 32. In the application configuration, composition 27 may be applied, for example, to the anogenital and/or urogenital region following appropriate insertion. In some embodiments, composition 27 may be expelled through exit orifice 32 by applying a force to container 29 (e.g., by squeezing container 29). As can be seen, nanosensor 24 is capable of communicating 43 with an external device. Following application, applicator 20 may be converted into storage mode by placing applicator portion 22 back into internal reservoir 26 and fully sealing closure 28. In some embodiments, nanosensor 24 is capable of communicating with an external device in storage and/or disposal configurations. In some embodiments, nanosensor 24 may communicate with a display located on application portion 22.

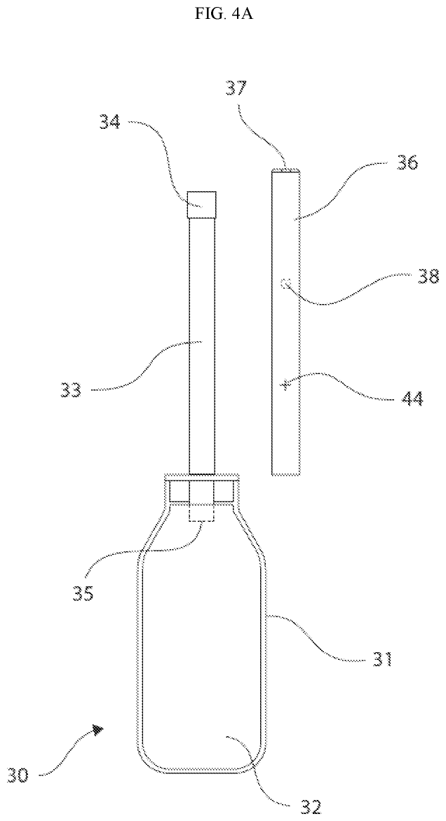

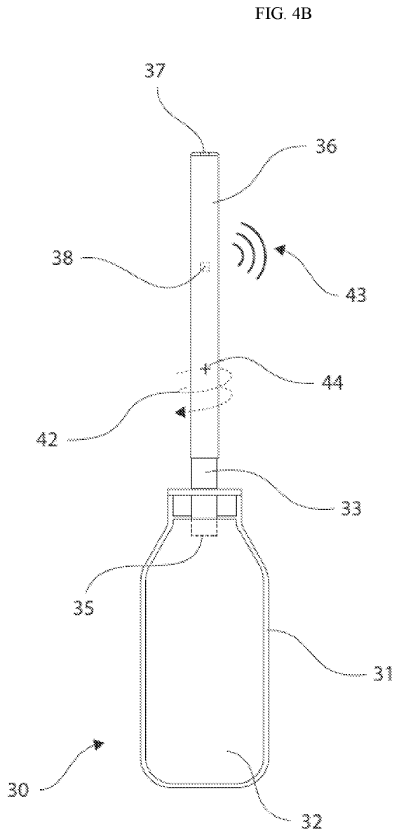

Referring now to FIGS. 4A and B, applicator 30 comprises a container 31 comprising an internal reservoir 32 capable of holding a composition. In some embodiments, the composition may be a composition of the present disclosure. Container 31 comprises a support 33 with distal end 34 which may be configured to be placed within internal reservoir 32 at support distal end 35. Within support 33 is a flow chamber in fluid communication with internal reservoir 32 running from distal end 35 to distal end 37. The applicator portion 36 (also referred to herein as a delivery element) with exit orifice 37 which may comprise sensor 38 (e.g., nanosensor 38) is dimensionally configured to be placed and removably attached over support 33 on container 31 as shown in FIG. 4B. Applicator portion 36 may be placed and secured on support 30 rotation 42 of applicator portion 36 with respect to container 31. Such securing of applicator portion 36 may occur via complimentary threading between support 30 and applicator portion 36. The sensor 38 may be configured to transmit pH information 43 to an external device and/or send pH information to a display 44. In some embodiments, the length of applicator portion 36 (i.e., the length along the major axis) is less than about 10 cm or less than about 9 cm or less than about 8 cm or less than about 7 cm or less than about 6 cm or less than about 5 cm. In some embodiments, the maximum circumference of applicator portion 36 is less than about 8 cm or about 7 cm or about 6 cm or less than about 5 cm or less than about 4 cm or less than about 3 cm. The internal reservoir may hold an amount of composition for a single use or have multiple uses. In some embodiments, the internal reservoir may have a volume of from about 5 mL to about 60 mL or about 10 mL to about 50 mL or about 15 mL to about 30 mL or about 20 mL to about 25 mL.

In some embodiments, the compositions of the present disclosure may be delivered using vaginal or topical films wherein the composition is capable of diffusing from the film into its surrounding environment. Suitable film formers include chitosan, hydroxypropyl methylcellulose and blends of these polymers (e.g., with 40% PEG 400 as plasticizer), a polymeric matrix/chitosan with carrageenan (κ-, λ, and ι-), pectin and gellan gum, hydroxyl propylcellulose and sodium alginate as polymers and propylene glycol and polyethylene glycol-400 as plasticizers, polyvinyl alcohol, poloxamer 407 and 188, hypromellose, sodium carboxymethylcellulose, hydroxylpropylmethylcellulose, hydroxyethylcellulose and polyvinyl pyrrolidone K-90, hydroxypropyl methylcellulose and Eudragit polymers (e.g., Eudragit RL100) and propylene glycol as plasticizer, hydroxypropyl methylcellulose, polyvinyl alcohol, polyethylene oxide, glycerol, poly(2-oxazoline)/polyoxazoline polymers and combinations thereof.

The applicators (or any portion thereof such as the container and/or the applicator element) may be formed from those materials known in the art. In some embodiments, portions of the applicator or the entire applicator may be made low waste packaging materials such as biodegradable plastics. Suitable biodegradable plastics may be bio-based plastics such as polyhydroxyalkanoates (PHAs), polylactic acid (PLA), starch blends, cellulose-based plastics, lignin-based polymer composites, and combinations thereof. The biodegradable plastics may also be petroleum based such as polyglycolic acid (PGA), polybutylene succinate (PBS), polycaprolactone (PCL), poly(vinyl alcohol) (PVA, PVOH), polybutylene adipate terephthalate (PBAT), and combinations thereof. In some embodiments, portions of the applicator (e.g., the applicator element) or the entire applicator may be composed of paper and/or cardboard. In some embodiments, the paper and/or cardboard applicators or portions thereof, may be burned or disposed of after vaginal mucosal contact, thereby decreasing risky medical waste.

The applicator may be composed of paper or cardboard. Each composition may be contained in an enclosure (e.g., slim bag, vial, or pouch), which may, in some embodiments, be a small, flexible bag configured to connect and fill an applicator, such as but not limited to a paper and/or cardboard applicator. The enclosure for the composition or gel described herein, may be composed of ethylene-vinyl acetate (EVA), polyethylene, low density polyethylene (LDPE), high density polyethylene (HDPE), or any biodegradable or recyclable material, or combinations thereof. Another embodiment provides for an enclosure that contains a composition having an acidic pH (e.g., pH 3.5-pH 5, pH 3.8-5). Yet another embodiment provides for an enclosure containing a composition having a neutral or alkaline pH or a pH from 6.5 to 7.4. The applicator may have a paper and/or cardboard barrel and plunger, and a mechanical stop and/or a fill line which assists in the prevention of overfilling the applicator in an amount greater than the intended dose when a user or other person who may fill the applicator with the desired composition. The fill line to which the composition should be added to the applicator without exceeding the fill line, may indicate the amount of composition for a single use, multiple use, e.g., 4.0-gram amount, combinations thereof, or any amount recommended for administration. However, different amounts or volumes may similarly be indicated by the fill line depending on the composition (e.g., acidic pH, neutral or alkaline pH), dosage, usage, etc. The composition enclosure, in some embodiments, may be a pharmaceutical grade pouch which allows preservative-free dosing, with high biocompatibility and low-leeching of resins. In one embodiment, the compositions of the disclosure may be stored in the enclosure under stressed (accelerated) conditions for three months and yet maintain the integrity of the compositions stored therein.

The applicators or methods of the present disclosure may include a system for the measurement of one or more parameters of the metabolomic profile of a biological sample (e.g., derived from saliva, blood, urine, feces, genital fluids, tears, nasal swabs, sweat, psoriasis lesions). These systems may comprise:
a substrate;
a sensor medium immobilized on said substrate comprising a microfluidic chip and/or a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile of the biological sample.

Typically, these systems operate using electric field as field effect transistors. Accordingly, the carbon nanostructure may exhibit a doping level or electronic shift upon interaction with a biological sample absorbed thereon. Such shifts may manifest in various changes to the electronic interaction of the carbon structure resulting in measurable electronic differences. For example, the electrical properties of the sensor may change in transconductance, threshold voltage shift, relative change in conductance at a specified voltage (e.g., from −1V to 1V), change in overall conductance when normalized to the threshold voltage, and the relative change in the minimum conductance, or combinations thereof as compared to the sensor medium not having the biological sample deposited thereon or in in situ contact. In some embodiments, deposition of the biological sample occurs through in situ direct or indirect contact with the sensing medium. Furthermore, these various changes in the electronic nature of the sensor medium may be used correlated (e.g., through linear discriminate analysis of the electronic changes to determine which variables are correlated with the metabolomic profile of the biological sample (and in particular, biological samples correlated with distinct bodily microbiomes such as cervical fluids)).

The plurality of carbon nanostructure may comprise one or more carbon nanotubes (e.g., single walled carbon nanotube) and/or graphene sheets (e.g., holed graphene). In some embodiments, the graphene comprises zigzag edge states. In some embodiments, the carbon nanostructure comprises armchair edge states. In some embodiments, the carbon nanostructure is oxidized.

Further tuning of the electronic properties may be achieved through deposition of various materials onto the plurality of carbon nanostructures. For example, the plurality of carbon nanostructures may have one or more conductive materials deposited thereon. In some embodiments, the conductive materials may be selected from one or more conductive polymers (e.g., polyaminoanthracene, polyaniline, polypyrrole) or one or more conductive metal (e.g., silver, gold, palladium, platinum) or combinations thereof. In some embodiments, the conductive material may comprise at least two conductive metals in the form of nanoparticles deposited on the plurality of carbon nanostructures. In certain implementations, the conductive material may comprise two or more forms of metal nanoparticles; wherein the forms of metal nanoparticles differ by surface functionalization. Such surface functionalization may comprise self-assembled monolayers of compounds having different head groups. The head groups may be independently selected from alkyl (e.g., C₁-C₃ alkyl such as methyl), —OH, —COOH, —SH, and —NRR; wherein R is independently selected at each occurrence from hydrogen and alkyl. In certain implementations, one form of metal nanoparticle comprises a self-assembled monolayer of dodecanthiol. In some embodiments, the form of metal nanoparticle comprises 11-mercaptounedecanoic acid.

As discussed above, different permutations and functionalization of the carbon nanomaterial may have effect on the electroscopic properties of the system. These can be measured and correlated with biological samples. For example, the measured electrical properties comprise change in transconductance, threshold voltage shift, relative change in conductance at a specified voltage (e.g., from −1V to 1V), change in overall conductance when normalized to the threshold voltage, and the relative change in the minimum conductance, or combinations thereof as compared to the sensor medium not having the biological sample deposited thereon. In some embodiments, the correlation between the one or more and the metabolomic profile has been determined by linear discriminant analysis (LDA).

These sensor mediums may also be configured to measure the pH of the biological sample based on the electrical properties of the sensor medium having the biological substance deposited thereon or in contact in situ.

The systems may involve the transmission of data to an external device. For example, in some embodiments, the correlation system comprises the transmission of the electrical property to an external device (e.g., laptop, tablet, computer, server, smart phone). In various implementations, the correlation system may comprise the transmission of data to an external device (e.g., laptop, tablet, computer, server, smart phone) configured to correlate the transmitted data into the one or more parameters of the metabolomic profile.

In certain implementations, the one or more parameters of the metabolomic profile is correlated with the number of one or more species of pathogenic bacteria present in the biological sample, the number of one or more species of beneficial bacteria present in the biological sample, similarity to a metabolomic profile of a biological sample from a healthy individual (e.g., eubiotic individuals, individuals without hrHPV), similarity to a metabolomic profile of a biological sample from an individual suffering a particular disease, disorder, or condition (e.g., dysbiosis, urethritis, bacterial vaginosis, hrHPV), or combinations thereof. The metabolomic profile may comprise, or relates to one or more biogenic amines such as cadaverine, putrescine, spermine, spermidine, agmatine, tyrosine, putrescince, tyramine, cadaverine, agmatine, N-acetylputrescine, carnitine, deoxycarnitine, pipecolic acid, pipecolate, lactate, tyrosine, sphingosine, adenine, guanine, xanthine, uric acid, caffeine, glutamate, phenylalanine, glutathione, glycylproline, or combinations thereof. In certain implementations, the metabolomic profile may comprise, or relate to, one or more amino acids and molecules similar thereto such as arginine, betaine, choline, lysine, methionine, ornithine, and S-adenosyl methionine and/or short chain fatty acids such as formate, acetate, propionate, butyrate, isobutyrate, valerate, and isovalerate and/or vitamins or vitamin-like compounds such as vitamin B2, vitamin B12, vitamin C, vitamin A, vitamin D, vitamin K, vitamin B3, and vitamin B5 and/or antioxidants such as glutathione. The sensor array may measure metabolomic profiles of those compounds which are produced chemically and/or microbiologically such as Vitamins A and C, those compounds which are produced in one or more microbial enzymatic step such as vitamins B3, B5, D, and K, compounds produced by bacterial fermentation (e.g., neurotransmitter compounds) such as γ-aminobutyric acid (GABA), 5-HT (serotonin), acetylcholine, dopamine and histamine. Several of these components such as Vitamins D, K, B3, and B5 For example, the sensors may identify differences the concentration of any one biogenic amines, amino acids and molecules similar thereto, short chain fatty acids, antioxidants, vitamin-like compounds, compounds produced chemically in the microbiome or microbiologically, compounds produced in one more microbial enzymatic steps, compounds produced by bacterial fermentation, or combinations thereof over time, or in comparison to a baseline or control. In some embodiments, the sensor may identify the concentration or changes concentration (e.g., with respect to a reference, a sample, a control, a baseline) of one or more of cadaverine, putrescine, spermine, spermidine, agmatine, tyrosine, putrescince, tyramine, cadaverine, agmatine, N-acetylputrescine, carnitine, deoxycarnitine, pipecolic acid, pipecolate, lactate, tyrosine, sphingosine, adenine, guanine, xanthine, uric acid, caffeine, glutamate, phenylalanine, glutathione, glycylproline, arginine, betaine, choline, lysine, methionine, ornithine, and S-adenosyl methionine, formate, acetate, propionate, butyrate, isobutyrate, valerate, and isovalerate, vitamin B2, vitamin B12, vitamin C, vitamin A, vitamin D, vitamin K, vitamin B3, and vitamin B5, or combinations thereof. In some embodiments, whether the microbiome needs further optimization may be determined based on sensing of specific metabolites associated with disease, disorder, or condition, such as the biomarkers of bacterial vaginosis described, for example, in T Nelson, frontiers in Physiology 6 (2015): Article 253, which is hereby incorporated by reference in its entirety and particularly in relation to the biomarkers and concentrations thereof such as those identified in FIGS. 1, 3, 5, 6, and Table 4. In some embodiments, the metalobolomic profile may be assessed In some embodiments, any metabolite such as those described herein may be measured in the form produced in situ, or as conjugate acids thereof, conjugate bases thereof, oxidized forms thereof, or reduced forms thereof.

In some embodiments, the measurement of the metabolomic profile of the biological sample may occur with one or more microfluidics devices as the sensing medium or in concert with the nanocarbon FET based devices described herein. The microfluidic sensing portion may comprise a microfluidic chip comprising: at least one channel, and a detector configured to detect signals of metabolites within the channel. These microfluidics devices may use colorimetric or electrostatic based measurement, such as those described in A Koh, et al., Sci Transl Med 8 (2016): 366dra165 which is hereby incorporated by reference in its entirety and particularly in relation to microfluidics designs and protocols. The microfluidic sensors may identify metabolites present in the biological sample such as biogenic amines, amino acids and molecules similar thereto, short chain fatty acids, vitamin-like compounds, compounds produced chemically in the microbiome or microbiologically, compounds produced in one more microbial enzymatic steps, compounds produced by bacterial fermentation, or combinations thereof over time, or in comparison to a baseline or control. In some embodiments, the microfluidics device may measure the concentration of metallic ions such as metal co-factor that may comprise zinc, selenium, manganese, molybdenum, cobalt, iron, copper, including salts thereof, or any combination thereof. Microfluidics devices may be based on chemiluminescent reactions with, for example, luminol based reactants, such as those described in C Provin, et al., IEEE Jour of Oceanic Engineering 38 (2013): 178-185, which is hereby incorporated by reference in its entirety and particularly in relation to chemiluminescent microfluidic sensing.

Devices for the measurement of one or more parameters of the metabolomic profile of a biological sample (e.g., derived from saliva, blood, urine, feces, genital fluids, tears, nasal swabs, sweat, psoriasis lesions) are also provided. These devices may comprise:
a) a handle portion dimensioned to be held in a user's hand comprising a power source (e.g., one or more batteries such as a lithium ion battery, one or more solar cells, USB connection, RFID); and
b) a sensor portion comprising one or more sensors; wherein the sensors comprise:
   a substrate;
   a sensor medium immobilized on said substrate comprising a microfluidics based sensor and/or a plurality of carbon nanostructures; wherein the plurality of carbon nanostructures has one or more conductive materials deposited thereon;
   at least two conductive terminals in electrical connection with the sensor medium and spaced from each other;
      wherein said sensor portion is removably attached to the handle portion; and when the sensor portion is attached to the handle portion, the one or more systems are in electrical communication with the power source; and
      said device comprises at least one measurement system to measure one or more electrical properties of the sensor medium when the sensor medium comprises the biological sample deposited thereon or in in situ contact therewith. In some embodiments, the power source is an electrical conduit between another device which provides the power required for measurement such as USB connections or RFID. In some embodiments, the device may be configured to transmit the electrical property to an external device (e.g., laptop, tablet, computer, server, smart phone). In various implementations, the device may further comprise a correlation system calibrated to correlate the measured electrical property with the one or more parameters of the metabolomic profile of the biological sample. In some embodiments, the device may comprise one or more of the systems described herein. For example, the device may comprise from 1-1000 (e.g., 1-500, 3-500, 3-100) sets of substrate, sensor medium, and electrical contacts each independently selected in terms of surface functionalization, carbon nanostructure type, substrate material, contact with electrodes and the like.

Kit

Yet other embodiments of the disclosure may provide for a kit or applicator dispensing carton described here. These kits or cartons may comprise any of the product applicators and the compositions described herein. The kit may be a self-contained carton providing product dispensing and waste containment capabilities. Advantages of the kit may include a design that accommodates a bulk supply (i.e., multi-unit supply) of applicators; discrete product dispensing and use for the user; low-waste production; containment and limited hazardous waste in community trash; and a decreased shipping volume.

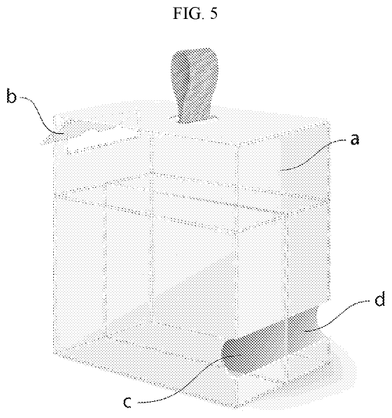

FIG. 5 shows an exemplary kit comprising: an upper waste receptacle (a); and lower portion comprising separate dispensing compartments (c) and (d), where the lower portion may be separated by a horizontal self from the upper waste receptacle. The waste receptacle (a) may comprise a barrier-lined flip top, where one side of the waste receptacle portion may have a punch open flap (b) through which applicators and composition containers or enclosures may be disposed of after use. In some embodiments, the waste receptacle (a) may be used to hold or safely contain waste products, including but not limited to the applicator, its components, and the gel or composition units. The waste receptacle may include an inner lining (e.g., a trash bag). Another embodiment may provide for a kit comprising a waste receptacle (a) that is sufficiently reinforced to allow for waste burning within the waste receptacle. This would be particularly useful for burning contaminated applicators disposed of in the upper waste receptacle portion. Another embodiment provides for kits composed of paper and/or cardboard, or some other material that is burnable for easy waste disposal, which would similarly be useful for disposing contaminated waste items collectively, such as by burning or incinerating. For example, after use of the supplied applicators, gel enclosures, and the like, the entire kit comprising these used items may be incinerated without any risk to the disposer of contracting any disease from the used items. In another embodiment, the entire kit may be burned and/or incinerated. The kit of the disclosure may comprise dispensing compartments (c) and (d) separated by a vertical wall, where the function of compartments (c) and (d) may be used interchangeably. One embodiment provides compartment (c) as a vaginal applicator dispensing compartment, and compartment (d) may be a gel or composition dispensing compartment comprising gel or composition units for use with the applicators in compartment (c). In an alternative embodiment, compartment (c) as a gel or composition dispensing compartment, and compartment (d) may be a vaginal applicator dispensing compartment. Yet another embodiment may provide for a kit having a lower portion comprising one compartment that holds and dispenses a pre-filled applicator containing a composition having an acidic pH (e.g., about pH 4-about pH 5) and another compartment that holds and dispenses a pre-filled applicator containing a composition having a neutral or alkaline pH (e.g., about pH 6.5- about pH 7.4). The kit may contain applicators, and associated components of the applicator and/or kit, that are composed of paper and/or cardboard, or another degradable material, allowing them to be easily and safely disposed of by burning or incinerating, thereby reducing waste, particularly biohazardous waste. Disposable and burnable products may be particularly useful in cases where the user has a sexually transmitted disease (STD) including but not limited to, HIV, HSV, HPV, hrHPV, and the like.

The gel or composition units or containers of the kit described here may be configured for use in filling the applicators with the gel or composition, e.g., designed to connect to the applicator. The units may be composed of a material that is disposable such that the unit or container may fit through the punch open flap (b) of the upper waste receptacle portion of the kit. For example, the unit may be an enclosure for the gel or composition that is easily compressed or compacted after use and disposed of through the open flap (b) for self-contained waste storage.

In some embodiments, the kit may contain or be configured to hold a supply of applicators for treatment of one month or greater (e.g., 2, 3, 4, 5, 6, 7, 8, 9, 10, 11, 12). The applicators may be initially empty for filling by the user, or pre-filled, with compositions of the disclosure. Other embodiments may provide for a kit comprising or configured to hold 50% or greater (e.g., 60%, 70%, 90%) applicators; or 50% or fewer (e.g., 40%, 25%, 10%) applicators, where the applicators contain compositions having an acidic pH (e.g., pH 4- pH 5). Yet another embodiment may be directed to a kit comprising or configured to hold 50% or greater (e.g., 60%, 70%, 90%) applicators; or 50% or fewer (e.g., 40%, 25%, 10%) applicators, where the applicators contain compositions having a neutral or alkaline pH (e.g., pH 6.5-pH 7.4).

Other embodiments may provide a kit containing 2 or more (e.g., 4, 6, 8, 10, 12, 14, 16, 18, 20, 22, 24, 26, 28, 30, 36, 40, 48, 50, 54, 56, 60, 68, 72, 78) applicators; 372 or fewer (e.g., 144, 140, 138, 132, 130, 126, 120, 114, 110, 108, 102, 100, 96, 90, 84, 78, 72, 66, 60, 54, 48, 42, 36, 30, 24, 18, 12, 6 or fewer) applicators; applicators in a range of 2-372 (e.g., 4-364, 6-288, 12-138, 18-132, 24-126, 30-120, 42-114, 48-108, 54-102, 60-96, 66-90, 72-84); or applicators in a sufficient number for a supply of one month or more (e.g., 2, 3, 4, 5, 6, 7, 8, 9, 10, 11, 12 months), where a one month supply may contain 24 or more (e.g., 25, 26, 27, 28, 29, 30, 31) applicators. The kit may provide for a waste containment receptacle and an easy and discrete applicator dispenser(s) with or without a gel or composition unit or enclosure dispenser. Other embodiments may be directed to a kit or carton that provides all product needs for the composition and its use, without external inputs (e.g., no water, batteries, or the like would be necessary).

The kit described here may also contain instructions for use and/or a dosing schedule. The dosing schedule may be included as a sticker on the external carton surface. In other embodiments, the kit may be of any sized dimensions sufficient to hold the desired number of applicators or composition units, and a waste receptacle for holding the used and disposed of applicators or composition units. The carton may be sufficiently large to hold a 1 month to 12 months' supply of the applicators and composition(s), for example a three months' supply. With respect to the dimensions of the kit, a kit designed to contain a close-packing ensemble of components is desirable. For example, where the front of the kit contains the openings of dispensers (c) and (d) and the back of the kit comprises the punch open flap (b) to the waste receptacle portion (a) as can be seen in FIG. 5, the kit dimensions may be, for example, about 3 to about 3.5 inches in width, about 6 inches in depth (from front to back), and about 6.5 inches in height. When shipping kits to populations or communities in need of the compositions described here, including those for treatment of STDs (e.g., HIV, HSV, HPV, hrHPV) and cervical cancer, a small kit dimension is desirable to reduce the costs associated with shipping. The packing volume of a kit for a 12 months' supply may be, for example, about 0.27 ft³, which is about 3-fold less than that of another commercially available carton (e.g., RepHresh: about 0.82 ft³) or about 1.3-fold less than that of a medium sized box (e.g., USPS: about 0.34 ft³). The kit may also contain a handle on the top external surface of the upper waste receptacle portion, where the handle may be composed of a paper, cardboard, plastic, reinforced plastic, fabric, reinforced fabric material, or any material similar to the aforementioned, or combinations thereof, which may support the weight of a kit containing the applicators, composition units, and waste receptacle with or without waste produced therefrom. For packing efficiency, the handle may be retractable or foldable to allow for multiple kits to be stacked one on top of another, thereby allowing for efficient use for storing or shipping. In some embodiments, the carton is compactable to minimize shipping volume and space and is readily collapsible after use, for self-contained disposal in the original carton.

### EXAMPLES

### Example 1: Foaming Wash & Genital Prebiotic Conditioner

Baseline pH levels in 6 circumcised white males ranging from 22-60 yrs of age were identified.

Left antecubital fossa samples were chosen as a control with no subjects having showered in the previous 10 hours or more prior to sample acquisition. From these samples, a pH (average±SD) of 4.52±0.52 was found for these subjects.

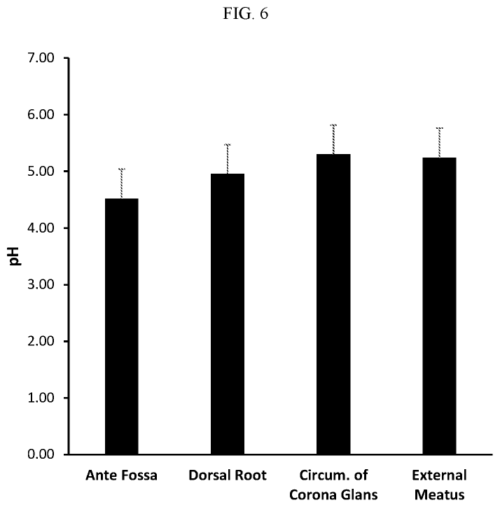

Penile pH levels at several positions were measured. Average pH values were 4.96 +/−0.52 (at the dorsal root); 5.30+/−0.52 (around the circumference of the corona of the glans penis); and 5.24+/−0.52 (over the external meatus). In 5 of the 6 men, penile pH was higher at the meatus than at the dorsal root. Penile pH levels, showed a gradient (P<0.05) towards a higher pH moving distally forward on the penis, with the glans and coronal sulcus and external meatus being a higher pH than the distal root of the penis (and the forearm control). The average pH values and the standard deviation at each measured position are shown in FIG. 6.

The impact of prebiotic genital care systems on pH at these locations was assessed using a penile foaming wash followed by a leave-on conditioning lubricant, applied to the penis (n=4). Leading commercial products, containing ingredients known to harm health microbiome species, were also measured. Specifically Nivea Men DEEP Active Clean (listed ingredients: Water, Sodium Laureth Sulfate, Cocamidopropyl Betaine, Acrylates Copolymer, PEG-7 Glyceryl Cocoate, Fragrance, Charcoal Powder, PEG-200 Hydrogenated Glyceryl Palmate, PEG-40 Hydrogenated Castor Oil, PEG-3 Distearate, Trisodium EDTA, Sodium Hydroxide, Phenoxyethanol, Methylparaben, Ethylparaben) followed by Astroglide lubricant (listed ingredients: Purified Water, Glycerin, Propylene Glycol, Polyquaternium 15, Methylparaben, Propylparaben) were compared to formulations of the disclosure. In these experiments, the components of the penile foaming wash and leave-on conditioning lubricant measured are shown in Table 32.

TABLE 32

| | Wash - | Conditioner - |
|---|---|---|
| | Formula A1 | Formula B1 |
| Ingredient | (wt %) | (wt %) |
| Purified Water, USP | | 98.972 |
| Deionized Water | 94.847 | |
| Sodium chloride | 0.100 | 0.300 |
| Lactulose | 0.050 | 0.050 |
| (prebiotic oligosaccharide) | | |
| Rosmarinus officinalis | 0.050 | |
| essential oil (essential oil | | |
| comprising bornyl acetate) | | |
| Abies sibirica essential oil | 0.020 | |
| (essential oil comprising | | |
| bornyl acetate) | | |
| Hypromellose | 0.300 | 0.400 |
| Sodium benzoate | 0.250 | |
| Gluconolactone | 0.750 | |
| Arabinogalactan | 0.100 | |
| Mentha spicata essential oil | 0.020 | 0.015 |
| (Biofilm inhibiting agent) | | |
| Citrus aurantium var. | 0.010 | 0.010 |
| amara essential oil | | |
| (flavonoid) | | |
| Manganese chloride | 0.0025 | 0.0025 |
| (Metal co-factor) | | |
| Sodium Cocyl Isethionate | 1.000 | |
| Coco Betaine (36% active) | 1.000 | |
| Citric Acid (buffering agent, | QS to pH 5.0 | |
| pH adjusting agent) | | |
| Bornyl acetate (not added | | 0.010 |
| from essential oils) | | |
| Monosodium phosphate, | | 0.240 |
| anhydrous (buffering agent) | | |
| Sodium hydroxide | | QS to pH 4.8 |
| (pH adjusting agent) | | |

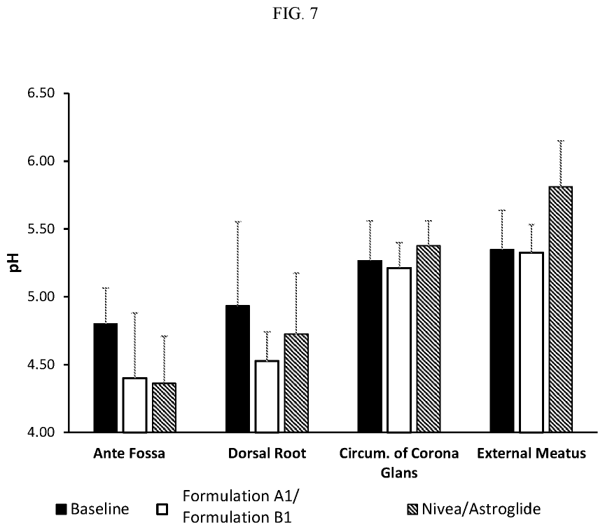

The impact on pH of Formula A foaming wash and Formula B as a leave on conditioner to the penis, were evaluated. Penile pH was measured at baseline, and after three days of treatment with each product. The results are shown in FIG. 7. Significant impacts of the treatment type on pH values were observed (p<0.05) for all measurements. The Nivea/Astroglide treatment over 3 days significantly raised penile pH levels (Mean+/−SD) as compared to the pH levels observed at baseline and after use of the prebiotic formulations Al/B1 at the external meatus. The commercial products also strongly raised penile pH (approaching pH 6; p<0.01) over that found at the unwashed antecubital fossa control.

Anerobic bacterial infections of the penis, especially from distal sites, have been associated with: vaginal infections in partners; and greater risk of STDs and HIV in men. Elevated penile pH levels may be associated with a reduction in healthy microbial species of the human genital organs. Application of products that elevate skin pH tends to destabilize the normal protective glycocalyx of skin. Other studies have shown an association between higher skin pH and disease. As can be seen, the compositions of the present disclosure have an effect of minimizing penile pH change following their application.

### Example 2: Formulations

Several wash and conditioning formulations have been prepared with the components as shown in Tables 33 (wash) and 34 (conditioner) and 35.

TABLE 33

| | Form. a1 | Form. a2 | Form. a3 |
|---|---|---|---|
| Ingredient Name | (% by wt.) | (% by wt.) | (% by wt.) |
| Deionized Water, USP | 95.645 | 91.227 | 94.108 |
| Sodium chloride | 0.1000 | 0.200 | 0.100 |
| Lactulose | 0.0500 | 0.100 | 0.750 |
| (prebiotic oligosaccharide) | | | |
| Picea mariana essential oil | 0.0200 | | |
| Hydroxyethyl cellulose | 0.4000 | | |
| Sodium dehydroacetate | 0.1500 | | 0.150 |
| Arabinogalactan | 0.1000 | 0.100 | 00 |
| Mentha spicata essential oil | 0.0200 | 0.020 | 0.020 |
| (biofilm inhibiting agent) | | | |
| Citrus paradisi essential oil | 0.0100 | | |
| (flavonoid) | | | |
| Manganese chloride | 0.005 | 0.0025 | 0.0015 |
| (metal co-factor)* | | | |
| Sodium Cocyl Isethionate | 1.500 | 1.250 | 1.500 |
| Cocamidopropyl Betaine^{†} | 2.000 | 6.300 | 1.000 |
| Citric Acid (buffering agent, | QS to | | QS to |
| pH modifying agent) | pH 5.0 | | pH 5.0 |
| Rosmarinus officinalis essential | | 0.050 | 0.050 |
| oil (essential oil comprising | | | |
| bornyl acetate) | | | |
| Cetyl hydroxyethylcellulose | | 0.600 | |
| Gycerol caprylate | | 0.025 | |
| Sodium benzoate | | 0.100 | |
| Pseudotsuga menziesii essential | | 0.015 | |
| oil | | | |
| Citrus aurantium var. amara | | 0.010 | 0.010 |
| essential oil (flavonoid) | | | |
| Lactic Acid (buffering agent, | | QS to | |
| pH modifying agent) | | pH 5.0 | |
| Abies sibirica essential oil | | | 0.01 |
| (essential oil comprising bornyl | | | |
| acetate) | | | |
| Hydroxypropyl guar gum | | | 0.300 |
| Polysorbate 20 | | | 2.000 |

*The indicated weight percentage of metallic co-factor (i.e., magnesium chloride) was achieved by adding the appropriate amoun of a hydrate of the metallic salt (e.g., magnesium chloride (II) tetrahydrate).
^{†}The indicated weight percent of Cocamiclopropyl betaine is the weight percent from a 36% solution of cocamidopropyl betaine added to each solution.

TABLE 34

| | Form. b1 | Form. b2 | Form. b3 |
|---|---|---|---|
| Ingredient Name | (% by wt.) | (% by wt.) | (% by wt.) |
| Purified Water, USP | 98.832 | 98.617 | 96.825 |
| Monosodium phosphate, anhydrous | 0.240 | | |
| Sodium chloride | 0.300 | 0.500 | 0.500 |
| Lactulose | 0.050 | 0.10 | 0.050 |
| (prebiotic oligosaccharide) | | | |
| Abies sibirica essential oil | 0.020 | | 0.020 |
| (essential oil comprising bornyl | | | |
| acetate) | | | |
| Hydroxyethyl cellulose | 0.400 | | |
| Mentha spicata essential oil | 0.015 | | |
| (biofilm inhibiting agent) | | | |
| Sodium Benzoate | 0.130 | | |
| Citrus aurantium var. amara | 0.010 | 0.010 | |
| essential oil (flavonoid) | | | |
| Manganese chloride | 0.003 | 0.003 | |
| (metal co-factor)* | | | |
| Lactic Acid (buffering agent, pH | QS to | | |
| modifying agent) | pH 4.8 | | |
| Rosmarinus officinalis essential oil | | 0.040 | |
| (essential oil comprising bornyl | | | |
| acetate) | | | |
| Pseudotsuga menziesii essential oil | | 0.015 | |
| Hydroxypropyl guar | | 0.550 | |
| Sodium dehydroacetate | | 0.150 | |
| Mentha aquatica essential oil | | 0.015 | |
| (biofilm inhibiting agent) | | | |
| Citric Acid (buffering agent, pH | | QS to | |
| modifying agent) | | pH 4.8 | |
| Hypromellose | | | 0.600 |
| Lactobacillus Ferment | | | 2.000 |
| Gluconolactone | | | 0.005 |
| Sodium hydroxide | | | QS to |
| (pH adjusting agent) | | | pH 4.8 |

*The indicated weight percentage of metallic co-factor (i.e., magnesium chloride) was achieved by adding the appropriate amount of a hydrate of the metallic salt (e.g., magnesium chloride (II) tetrahydrate).

Several compositions were formulated with the components as shown in Table 35.

TABLE 35

| | Formula Da | Formula Db | Formula Dd |
|---|---|---|---|
| Ingredient Name | (% by wt) | (% by wt) | (% by wt) |
| Purified water, USP | 97.576 | 97.274 | 98.067 |
| Monosodium phosphate, | | 0.523 | 0.240 |
| anhydrous | | | |
| Disodium phosphate | 0.531 | 0.325 | |
| Lactic acid | 0.500 | | |
| Sodium chloride | 0.200 | 0.100 | 0.300 |
| Lactulose | 0.050 | 0.050 | 0.050 |
| Abies sibirica | 0.015 | | |
| Xanthan gum | 0.900 | | |
| Mentha spicata | 0.015 | 0.050 | 0.015 |
| Rosmarinus officinalis | 0.010 | | |
| Sodium dehydroacetate | 0.200 | | |
| Manganese chloride* | 0.003 | 0.003 | 0.003 |
| Sodium hydroxide | QS to pH 4.5 | QS to pH 4.5 | QS to pH 4.5 |
| Pseudotsuga menziesii | | 0.01 | |
| Kappa-Carrageenan | | 1.000 | |
| Gluconolactone | | 0.250 | |
| Cetyl hydroxyethylcellulose | | 0.40 | |
| Citrus aurantium var. | | 0.015 | 0.010 |
| Amara | | | |
| Picea mariana | | | 0.015 |
| Hydroxyethyl cellulose | | | 1.100 |
| Arabinogalactan | | | 0.050 |
| Sodium benzoate | | | 0.15 |

*The indicated weight percentage of metallic co-factor (i.e., magnesium chloride) was achieved by adding the appropriate amount of a hydrate of the metallic salt (e.g., magnesium chloride (II) tetrahydrate).

### Example 3: Measurements on Post-Menopausal Women

Several vaginal wash and lubricant recipes of the present disclosure (Formula B2, Formula D 71519C) using either pure bornyl acetate or in an essential oil, when used in two post-menopausal women (ages 59 and 46), both undergoing hormone replacement therapy (HRT), have had the surprising effect of substantially:

1) increasing arousal and decreased latency to orgasm as compared to no treatment or treatment with commercially available products such as Astroglide®, K-Y®, and Replens®; and

2) long term lubrication, hydration and comfort as compared to no treatment or treatment with commercially available products such as Astroglide®, K-Y®, and Replens®.

Parameters were measured as disclosed in DL Rowland, et al., J Sex Med 15 (2018): 1463-1471, A. Huang et al., Menopause 17 (2010): 121-126, E. Erekson et al., Menopause: The Journal of the North American Menopause Society 20 (2013): 973-979, B. Ettinger, Menopause 15 (2008): 889-889, E. Gerstenberger, et al., Jour of Sexual Med 7 (2010): 3096-3103, each hereby incorporated by reference in their entirety.

### Example 4: Effect of Compositions on Mouse In-Vitro Fertilization and Embryo Development (IVF-MEA)

Two topical isotonic compositions were prepared with the components shown in Table 36. Formula A had a total bornyl acetate content of 0.02% by weight of the composition.

TABLE 36

| | Formula D1 | Formula A |
|---|---|---|
| Ingredient Name | (% by wt) | (% by wt) |
| Solvent | 98.077 | 98.019 |
| Buffering agent | 0.240 | 0.483 |
| Isotonicity agent | 0.300 | 0.100 |
| Prebiotic oligosaccharide (combination | 0.300 | 0.300 |
| of Lactulose and gluconolactone) | | |
| (−)-Bornyl acetate | 0.005 | |
| Essential oil comprising bornyl acetate | | 0.020 |
| (Abies sibrica extract) | | |
| Viscosity-increasing agent | 1.000 | 1.000 |
| Humectant | 0.050 | 0.050 |
| Biofilm inhibiting agent | 0.015 | 0.015 |
| Flavonoid | 0.010 | 0.010 |
| Manganese chloride* | 0.003 | 0.003 |
| pH adjusting agent | QS to pH 4.5 | QS to pH 4.5 |

*The indicated weight percentage of metallic co-factor (i.e., magnesium chloride) was achieved by adding the appropriate amount of a hydrate of the metallic salt (e.g., magnesium chloride (II) tetrahydrate).

10% vol/vol concentrations of the compositions in Table 36 were prepared in in vitro fertilization (IVF) media. Mouse oocytes were placed with mouse sperm in Formula A for 4 hours. After 4 hours of incubation at 37° C. and 5.0% CO₂, 21 fertilized mouse oocytes were washed and transferred to culture medium for 96 hours at 37° C. and 5.0% CO₂. Results following exposure to the 10% formulation solution are compared to mouse ova placed with sperm and fertilized in a control culture medium for 4 hours. After 4 hours of incubation at 37° C. and 5.0% CO₂, 21 fertilized mouse oocytes were washed and transferred to culture medium for 96 hours at 37° C. and 5.0% CO₂. Four-hour incubation with formula D6 resulted in 83% of oocytes fertilized, compared to 96% of control. 100% of fertilized oocytes exposed to A and to control developed to blastocysts.

To be considered non-toxic, the percent of oocytes fertilized within 4 hours in the formulation group should be 80% or more of that found in the control group, and the percent of oocytes developing to expanded blastocyst at 96 hours in the formulation group must not be less than 80% of that found in the control group. The results for the 10% concentration for 4 hour exposure test are shown in Table 37. As can be seen, Formula A is non-toxic.

TABLE 37

| | | | Oocytes to expanded |
|---|---|---|---|
| | Oocytes fertilized | Oocytes to 2-cell | blastocyst within |
| | within 4 hours | within 24 hours | 96 hours |
| Control | 96% | 100% | 100% |
| Formula A | 83% | 100% | 100% |
| | (86% of control) | | |

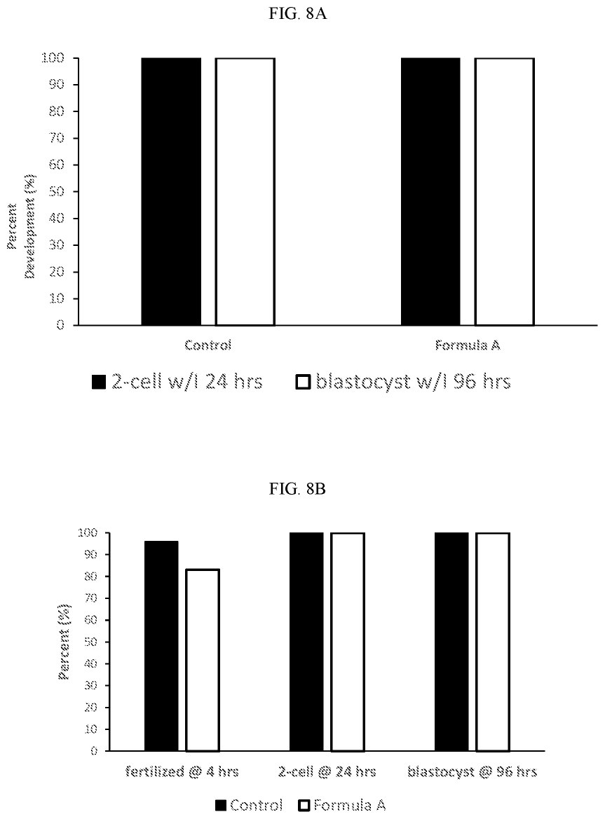

The effect of Formula A (Table 2) on Mouse Embryo Development (MEA) was assessed by exposing fertilized mouse embryo to a 10% concentration of Formula A for 30 minutes. (pH 4.5, preservative-free). The formulation demonstrated no detrimental effect on mouse embryo (FIG. 8A).

The effect of Formula A on in-vitro fertilization and embryo development in mice (IVF-MEA) was assessed. Mouse embryos and sperm were incubated together for 4 hours with a 10% conc. of Formula A (pH 4.5, preservative-free) or to control media. Gametes exposed to Formula A demonstrated an 83% fertilization rate versus 96% for control (86% of control). All fertilized embryos from both treatments developed to the blastocyst stage at 96 hours suggesting no toxicity (FIG. 8B).

### Example 5: Effect of Compositions on Mouse Embryo Development (MEA)

10% vol/vol concentrations of the compositions in Table 36 were prepared in M2 culture media. One-cell mouse embryos were placed in the Formula A for 30 minutes or in culture media alone (control) at 37° C. and 5.0% CO₂. The embryos were then then transferred to culture media and incubated for 96 hours at 37° C. and 5.0% CO₂. Results following one-cell mouse embryo exposure to the 10% formulation solution are compared one-cell mouse embryo in a control culture medium for 30 minutes. Incubation for 30 minutes resulted in 100% of the oocytes undergoing division within 24 hours and 100% of oocytes converting to expanded blastocyst within 96 hours for both control and test settings.

To be considered non-toxic, the number of oocytes developing to expanded blastocyst at 96 or 120 hours in the formulation group must not be less than 80% of that found in the control group. The results for the 10% concentration for 4 hour exposure test are shown in Table 38. As can be seen, Formula A is non-toxic.

TABLE 38

| | 1-cell embryos to | 1-cell embryos to |
|---|---|---|
| | 2-cell embryos | expanded blastocyst |
| | within 24 hours | within 96 hours |
| Control | 100% | 100% |
| Formula A | 100% | 100% |

### Example 6: Bovine Sperm Motility Studies

Commercial cryopreserved sperm from 2 bulls was pooled and used in all replicates. Semen straws (0.5 mL) were thawed and sperm concentration was adjusted to 10 million spermatozoa per mL using a commercial bovine semen-freezing medium.

Straws containing sperm were thawed for use in studies, where they were mixed with 10% gel treatment with one of the gel compositions in Table 39 and incubated for 30 min at 39° C. in a CO₂ incubator. The bornyl acetate content (coming from essential oils comprising bornyl acetate) for each formulation is also provided in Table 39.

TABLE 39

| | 71519A | 71519B | 71519C | 0618CA45 | 0618CA68 |
|---|---|---|---|---|---|
| | (% by wt) | (% by wt) | (% by wt) | (% by wt) | (% by wt) |
| Total bornyl acetate content | 0.045 | 0.025 | 0.055 | 0.02 | 0.02 |
| Ingredient Name | | | | | |
| Purified water, USP | 97.344 | 97.264 | 97.234 | 97.739 | 97.739 |
| Disodium phosphate | 0.531 | 0.531 | 0.531 | 0.531 | 0.531 |
| Lactic acid | 0.500 | 0.500 | 0.500 | | |
| Sodium chloride | 0.100 | 0.100 | 0.100 | 0.100 | 0.100 |
| Lactulose | 0.0500 | 0.050 | 0.050 | 0.050 | 0.050 |
| Abies sibirica | 0.0250 | 0.025 | 0.025 | 0.020 | 0.020 |
| Hypromellose | 0.500 | 0.500 | 0.500 | 0.400 | 0.400 |
| Gluconolactone | 0.500 | 0.500 | 0.500 | 0.100 | 0.100 |
| Hydroxypropyl guar gum | 0.400 | 0.400 | 0.400 | 0.400 | 0.400 |
| Rosmarinus officinalis | 0.020 | | 0.020 | | |
| Mentha spicata | 0.010 | 0.010 | 0.010 | 0.008 | 0.008 |
| Citrus aurantium var. | 0.015 | 0.015 | 0.015 | | |
| Amara | | | | | |
| Manganese chloride* | 0.005 | 0.005 | 0.005 | 0.00455 | 0.0054 |
| Arabinogalactan | | 0.100 | 0.100 | 0.200 | 0.200 |
| Picea mariana | | | 0.010 | | |
| Sodium hydroxide | QS to | QS to | QS to | QS to | QS to |
| | pH 4.8 | pH 4.8 | pH 4.8 | pH 4.8 | pH 4.8 |
| Citric acid | | | | 0.409 | 0.409 |
| Oleuropein | | | | 0.0200 | 0.0200 |
| Citrus reticulata | | | | 0.0100 | 0.0100 |
| Juniperus communis | | | | 0.0080 | 0.0080 |

*The indicated weight percentage of metallic co-factor (i.e., magnesium chloride) was achieved by adding the appropriate amount of a hydrate of the metallic salt (e.g., magnesium chloride (II) tetrahydrate).

After incubation and thorough mixing of samples, replicate aliquots were removed from treatments for computer assisted sperm analysis (CASA) using a Hamilton Thorne IVOS analyzer. Briefly, for each replicate, a 5 μL sample is loaded on a MicroCell sperm counting chamber. The CASA stage is set at 39° C. for analysis. A minimum of 5 fields per sample are analyzed for measurement of sperm motility and concentration. Analysis includes % motile sperm, % progressively motile sperm, and total motile sperm concentration, as well as numerous measurements of sperm motion including velocity and lateral head displacement. Sperm motility parameters following exposure to the test-item for 30 minutes must be ≥80% of control.

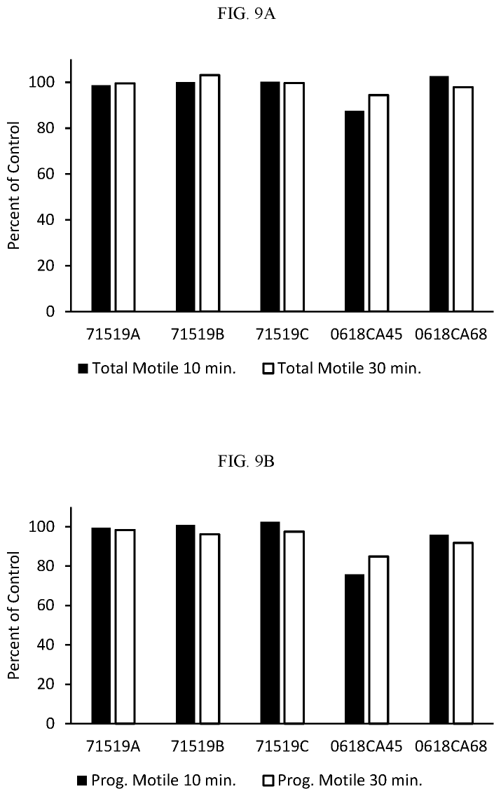

Generally, motility at 80% or greater is viewed as acceptable by the FDA. Total percent of motile sperm at 10 and 30 min and total percent progressively motile sperm at 10 and 30% were determined. Results for total percent motile sperm are shown in FIG. 9A and total percent for progressively motile sperm are shown in FIG. 9B. As can be seen, the addition of citric acid at pH 4.5 or 4.8 had a significantly negative deleterious impact on sperm function. With this exception, all formulas supported high levels of sperm total motility and progressive motility, with some formulas exhibiting sperm function equal to or greater than that of controls.

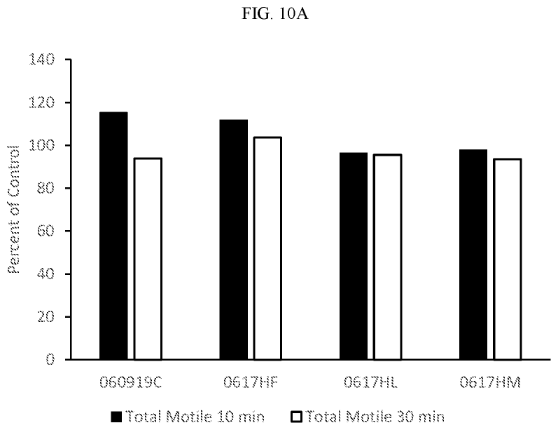

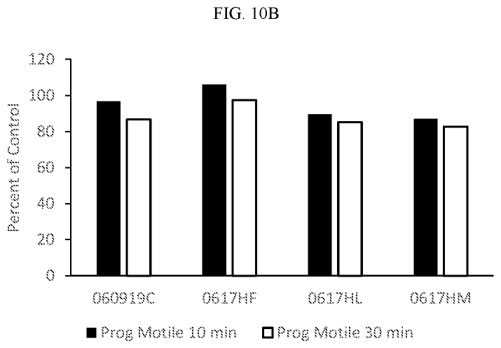

Formulations with a pH of 6.8 were also prepared with the components as shown in Table 40 and motility measurements were performed. The motility measurement results for these compositions are shown in FIGS. 10A and 10B.

TABLE 40

| | 060919C | 0617HF | 0617HL | 0617HM |
|---|---|---|---|---|
| Ingredient Name | (wt %) | (wt %) | (wt %) | (wt %) |
| Purified Water, USP | 96.9744 | 97.109 | 97.0285 | 97.1235 |
| hypromellose | 0.6 | 0.6 | 0.6 | 0.6 |
| Disodium phosphate, | 0.531 | 0.531 | 0.531 | 0.531 |
| anhydrous | | | | |
| gluconolactone | 0.5 | 0.56 | 0.56 | 0.56 |
| lactic acid | 0.5 | 0.5 | 0.5 | 0.5 |
| Hydroxypropyl guar | 0.4 | 0.4 | 0.4 | 0.4 |
| gum | | | | |
| Arabinogalactan | 0.2 | 0.2 | 0.2 | 0.2 |
| Sodium hydroxide | 0.19 | 0.206 | 0.16 | 0.208 |
| Sodium chloride | 0.1 | 0.05 | 0.05 | 0.05 |
| Lactulose | 0.05 | 0.0025 | 0.1 | 0.0025 |
| Abies sibirica | 0.02 | 0.025 | 0.008 | 0.008 |
| Citrus paradisi | 0.01 | 0.01 | 0.01 | 0.01 |
| Mentha spicata | 0.01 | 0.01 | 0.01 | 0.01 |
| Manganese chloride* | 0.0046 | 0.0025 | 0.0025 | 0.005 |
| pH | 6.8 | 6.8 | 6.8 | 6.8 |

*The indicated weight percentage of metallic co-factor (i.e., magnesium chloride) was achieved by adding the appropriate amount of a hydrate of the metallic salt (e.g., magnesium chloride (II) tetrahydrate).

Motility measurements were also performed on the compositions shown in Table 41. Composition 1004BA10 is also known as PL 1206 or PL102345.

TABLE 41

| | 1004BA5 | 1004BA10 | 1004BA15 |
|---|---|---|---|
| Ingredient Name | % by wt. | % by wt. | % by wt. |
| Purified Water, USP | 98.077 | 98.072 | 98.067 |
| Monosodium phosphate, | 0.240 | 0.240 | 0.240 |
| anhydrous | | | |
| Sodium chloride | 0.300 | 0.300 | 0.300 |
| Lactulose | 0.050 | 0.050 | 0.050 |
| Bornyl acetate | 0.005 | 0.010 | 0.015 |
| Hypromellose | 0.600 | 0.600 | 0.600 |
| Gluconolactone | 0.250 | 0.250 | 0.250 |
| Hydroxypropyl guar gum | 0.400 | 0.400 | 0.400 |
| Arabinogalactan | 0.050 | 0.050 | 0.050 |
| Mentha spicata | 0.015 | 0.015 | 0.015 |
| Neroli oil | 0.010 | 0.010 | 0.010 |
| Manganese chloride* | 0.003 | 0.003 | 0.003 |
| Sodium hydroxide | QS to pH 4.5 | QS to pH 4.5 | OS to pH 4.5 |

*The indicated weight percentage of metallic co-factor (i.e., magnesium chloride) was achieved by adding the appropriate amount of a hydrate of the metallic salt (e.g., magnesium chloride (II) tetrahydrate).

The compositions disclosed in Table 41 were measured to have effect on sperm motility as shown in Table 42.

TABLE 42

| | 1004BA5 | 1004BA10 | 1004BA15 |
|---|---|---|---|
| | % of control | % of control | % of control |
| Total Motile (10 min) | 109 | 89 | 98 |
| Total Motile (30 min.) | 90 | 87 | 97 |
| Progressively Motile (10 min.) | 109 | 82 | 93 |
| Progressively Motile (30 min.) | 97 | 85 | 100 |

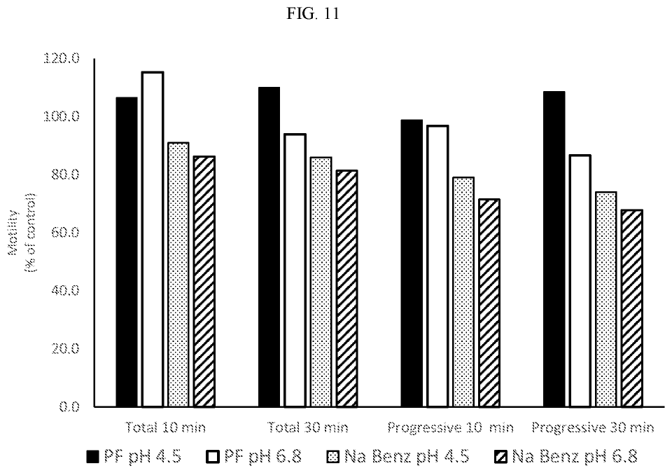

Bovine sperm were also incubated with 10% concentrations of high and low pH formulations with and without preservatives. Formulations at pH 4.5 outperformed formulations at pH 6.8 whether preservative-free (PF) or containing the preservative sodium benzoate (Na Benz) 0.19%. FIG. 11 illustrates these effects for preservative a free formulation at pH 4.5 (black bars, Formula A, Table 2), a preservative free formulation at pH 6.8 (white bars, Formula B, Table 3); a pH 4.5 formulation comprising 0.19% sodium benzoate by weight of the composition (dotted bars, Formula C, Table 4), and a pH 6.8 formulation comprising 0.19% sodium benzoate by weight of the composition (diagonally striped bars, Formula D, Table 5).

Growth of certain organisms in the compositions was also tested. For these measurements, the test microorganisms are prepared by growth in liquid or on agar culture medium. Suspensions of test microorganisms were standardized and the bacterial and fungal were pooled. The test and control substances were dispensed, in identical volumes to sterile vessels and then separately inoculated with the pooled microorganisms followed by incubation. Control substances were immediately harvested and represent the concentration present at the start of the test, or time zero. At the conclusion of each contact time incubated test substances are harvested, mixed, chemically neutralized, then assayed for surviving microorganisms. The number of surviving microorganisms at each contact times was assessed, and then microbial reductions were calculated based on initial microbial concentrations.

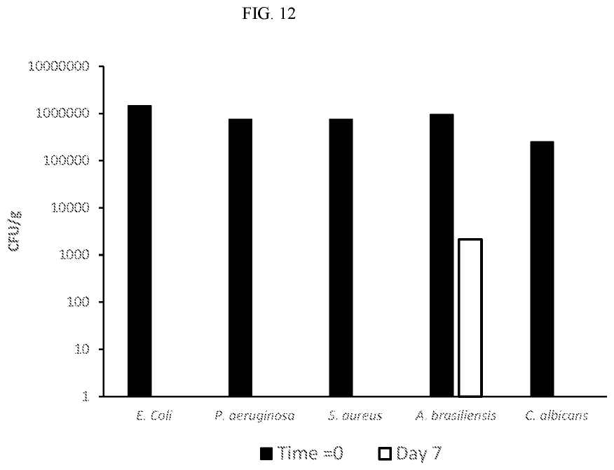

The preservative containing formula G (sodium benzoate 0.125%, Table 6) demonstrated very good preservative efficacy in a 7-day screen. Pathogenic bacteria and C. albicans demonstrated no growth at 7 days. A. brasiliensis growth was decreased by approximately 2.64 log (FIG. 12). As can be seen in FIG. 11, despite the observation that lower pH generally works synergistically to improve the efficacy of preservatives, presence of the preservative conversely had minimal if any effect on sperm at lower pH. Moreover, compositions having preservatives such as sodium benzoate at low concentrations (e.g., 0.125% by weight of the composition as in Formula E; Table 6) are effective in preventing microbial growth (FIG. 12).

### Example 7: Human Sperm Motility Measurements

A semen sample was obtained from a healthy normospermic donor. The specimen was produced by masturbation without lubricant into a sterile plastic container after a recommended abstinence period of 48-96 hours. The specimen was allowed to liquefy and then processed within 30 minutes. Spermatozoa were separated from liquefied semen by density gradient separation. The washed sperm were then resuspended in sperm wash medium. Formula D was added to an aliquot of the sperm sample to achieve a 10% V/V concentration. An aliquot from the same sperm sample but without Formula A, serves as the medium-only control. The samples were incubated in the same incubator at 32° C. and 5% CO₂ for 24 hours. Sperm motility following exposure to the test-item for 30 minutes must be ≥80% of control to be considered non-toxic. The sperm motility measurements initially and after 24-hours are shown in Table 43.

TABLE 43

| | Initial Motility | 24-hour motility |
|---|---|---|
| Control | 97% | 97% |
| Formula A | 97% | 97% |
| | (100% of control) | (100% of control) |

### Example 8: Human Sperm Survival Assay

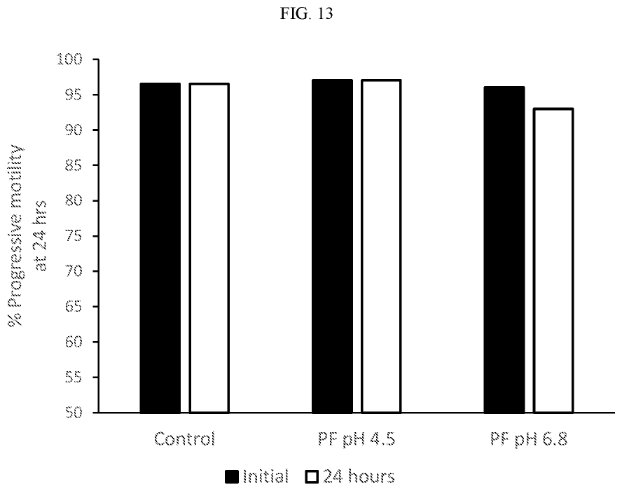

Human sperm were assayed using the survival assay disclosed in J Vargas Fertil Steril 95 (2011): 835-836, which is hereby incorporated by reference in its entirety, and particularly in relation to assays for measurements of the sperm toxicity of compositions such as gels. Briefly, human sperm were incubated with a 10% concentration of preservative-free formulations at pH 4.5 (Formula A, Table 2) and pH 6.8 (Formula B, Table 3) for 24 hours. Formulations at both pH levels were not harmful to sperm at 24 hours as can be seen in FIG. 13.

Human sperm were also incubated with formulations containing the preservative combination of Lactobacillus ferment (LF):sodium dehydroacetate (SDHA) at concentrations of 2%:0.075% (Formula G, Table 8) and 1.75%:0.056% by weight. The preservative-free formulations had no effect on progressive forward motility at 24 hours compared to control. The preservative combination reduced motility at 24 hrs in a dose dependent manner. Nevertheless, motility remained above 75% of control in all formulations indicating that they were “non-toxic” to sperm. The percent progressive motility for each formulation is shown in FIG. 14.

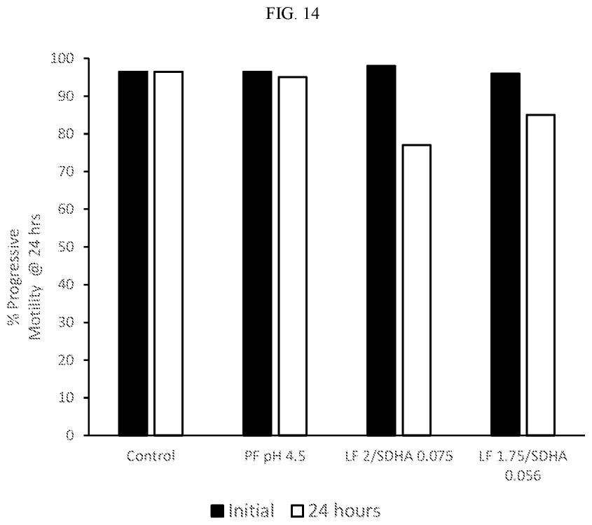

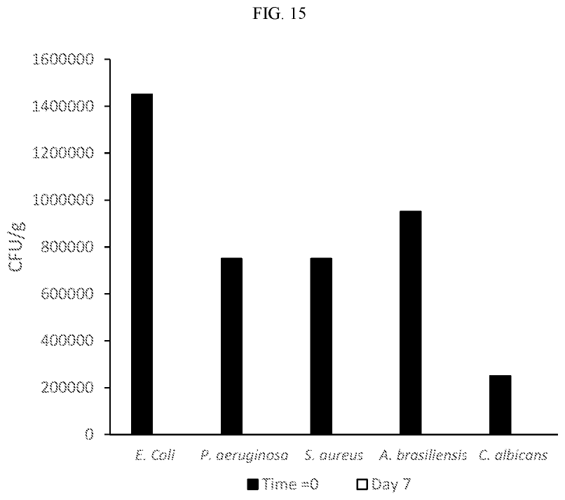

Additionally, the preservative assay as performed in Example 6 was also performed on the composition comprising Lactobacillus Ferment 2%:SDHA of 2%:0.075% by weight (Formula G, Table 8). Formula G also demonstrated excellent preservative efficacy as shown in FIG. 15. As can be seen, no colony forming units of pathogenic species were measured at day 7 in the compositions.

### Example 9: Gel Bactericidal Effects

The bactericidal effects of Formula H (Table 9 and Table 44) gel, a conditioning moisturizer (Table 44), a foaming cleanser (Table 44), and two commercially available gels (Trimosan and McKesson Perineal Cleanser Rinse Free) were studied.

TABLE 44

| | | Conditioning | |
|---|---|---|---|
| | Formula H | Moisturizer | Foaming Cleanser |
| | (wt %) | (wt %) | (wt %) |
| Deionized water | | | q.s. |
| Purified water | q.s. | q.s. | |
| Sodium hydroxide | To pH 4.5 | To pH 4.25 | To pH 5.0 |
| Sodium chloride | 0.300 | 0.400 | |
| Monosodium | 0.240 | 0.240 | |
| phosphate | | | |
| Lactulose | 0.050 | 0.050 | 0.0500 |
| Abies sibirica | 0.020 | 0.020 | |
| (Siberian fir) | | | |
| Hypromellose | 0.600 | 0.600 | |
| Lactobacillus Ferment | | 1.750 | 1.7500 |
| Sodium | | 0.056 | 0.0560 |
| dehydroacetate | | | |
| Gluconolactone | 0.500 | 0.500 | 0.5000 |
| Hydroxypropyl guar | 0.400 | | |
| gum | | | |
| Mentha spicata | 0.010 | 0.015 | 0.0200 |
| (spearmint) | | | |
| Neroli oil | 0.010 | 0.010 | 0.0100 |
| Manganese chloride* | 0.005 | 0.005 | 0.0025 |
| Rosemary | | | 0.0250 |
| Cetyl | | | 0.3000 |
| hydroxyethylcellulose | | | |
| Polysorbate 20 | | | 1.6000 |
| Arabinogalactan | 0.050 | | 0.1000 |
| Sodium cocoyl | | | 0.5000 |
| glutamate (37.5 active) | | | |
| Sodium | | | 0.5000 |
| lauroamphoacetate | | | |
| (30%) | | | |

*The indicated weight percentage of metallic co-factor (i.e., magnesium chloride) may be achieved by adding the appropriate amount of a hydrate of the metallic salt (e.g., magnesium chloride (II) tetrahydrate).

The effect of each neat gel as well as gels diluted with bacterial culture broth (Peptone Yeast Glucose Tween (PYG TWEEN FA/GLC), from Anaerobe Systems Morgan Hill, Calif. 95037) was tested by mixing it with equal parts of bacterial suspension at a standardized concentration adjusted to provide a good growth curve within a testing period of 4-24 h. At least two experiments were performed with each test agent where the agent was used undiluted an applied on two replicate cultures. Formula H (Table 9) was tested undiluted in four experiments. Serial dilutions of the gels were tested in one experiment for Formula H and 2 experiments for each of the other test products, each dilution tested in triplicate. The commercial products were tested neat each in one experiment and in triplicate cultures.

After each desired time period of incubation with the gels (0 time point, 4 h, and 24 h) each experimental mix was plated in serial dilutions on Brucella agar and bacteria were assessed for viability by enumeration of colony forming units (CFU) after a 2 to 4 days incubation under anaerobic conditions as described.

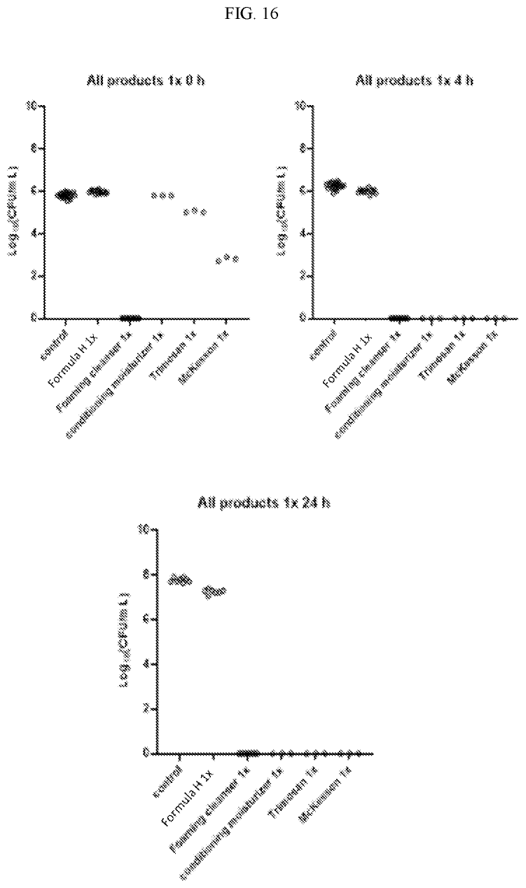

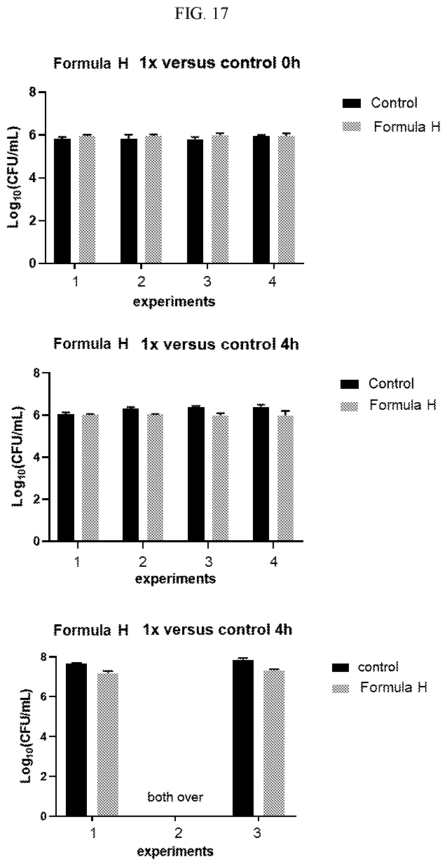

Lactobacillus recovery for each gel at three time points is shown in FIG. 16. Formula H resulted in satisfactory recovery of live L. crispatus from Formula H at all time points. Additionally, the recovery of L. crispatus following exposure to Formula H was not substantially different from recovery in the absence of gel (<0.5 log CFU difference) as shown in FIGS. 16-17 and Tables 45 (t=0), 46 (t=4 hr), and 47 (t=24 hr). The recovery was comparable to that of the growth medium specifically designed to support Lactobacillus indicated growth supporting properties. Formula H performed significantly better compared to the two commercial products, which were bactericidal within 4 h. Also, no live bacteria were recovered from the undiluted cleanser and conditioning moisturizer at 24 hours.

TABLE 45

| Experiment | Control | | | | Formula H | | | |
|---|---|---|---|---|---|---|---|---|
| (t = 0) | (log CFU/mL) | | | | (log CFU/mL) | | | |
| 1 | 5.79 | 5.78 | 5.8 | 5.96 | 5.95 | 5.9 | 6.04 | 5.97 |
| 2 | 5.87 | 5.9 | 5.99 | 5.54 | 5.88 | 6.05 | 5.94 | 5.96 |
| 3 | 5.72 | 5.73 | 5.9 | 5.89 | 6.01 | 6.1 | 5.88 | 6.01 |
| 4 | 6 | 5.88 | 5.95 | | 6.01 | 6.06 | 5.85 | |

TABLE 46

| Experiment | | | | | Formula H | | | |
|---|---|---|---|---|---|---|---|---|
| (t = 4 hr) | Control | | | | (log CFU/mL) | | | |
| 1 | 5.92 | 6.02 | 6.09 | 6.13 | 5.99 | 5.95 | 6.08 | 5.99 |
| 2 | 6.36 | 6.24 | | | 6.05 | 6.04 | | |
| 3 | 6.44 | 6.41 | 6.33 | 6.38 | 5.93 | 6.09 | 5.91 | 6.07 |
| 4 | 6.4 | 6.5 | 6.3 | | 6.2 | 5.8 | 6 | |

TABLE 47

| Experiment | | | | Formula H | | |
|---|---|---|---|---|---|---|
| (t = 24 hr) | Control | | | (log CFU/mL) | | |
| 1 | 7.63 | 7.69 | | 7.05 | 7.21 | 7.23 |
| 2 | overgrown | | | overgrown | | |
| 3 | 7.9 | 7.9 | 7.7 | 7.3 | 7.3 | 7.4 |
| 4 | overgrown | | | overgrown | | |

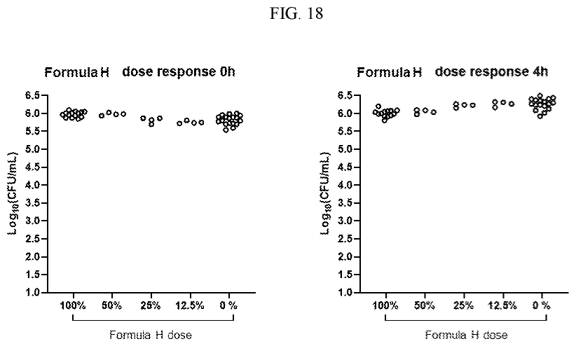

To illustrate the beneficial effect observed with Formula H, serial dilutions of the gel were performed and the Lactobacillus recovery was measured for each dilution at 0 hrs and 4 hrs. FIG. 18 shows the log CFU/mL for each dilution. These results confirm that Formulation H at all concentrations supported the growth of L. crispatus similarly to the bacterial culture broth suggesting prebiotic qualities of the formulation.

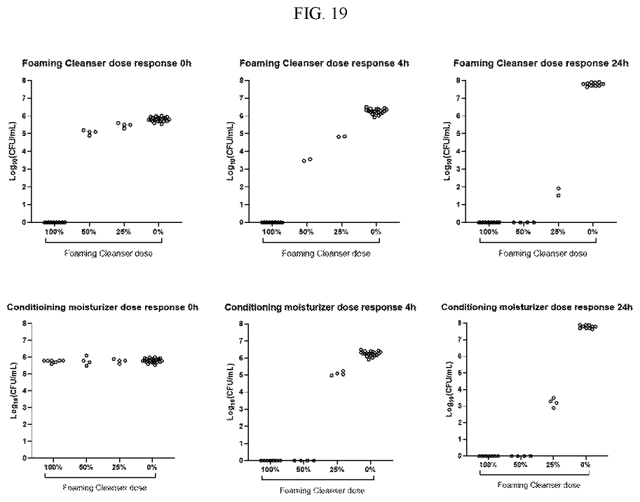

When applied undiluted, the Foaming Cleanser and Conditioning Moisturizer formulas decreased bacterial growth compared to the control medium. However, the growth of L. crispatus was partially restored in a dose and time-dependent manner as shown in FIG. 19. A 4× dilution was able to partially remediate the bactericidal effects indicates that optimization of these formulas may be possible.

### Example 10: Gamete Biocompatibility Testing

Cervical mucus penetration studies of bull sperm exposed to 10% concentrations of compositions of the present disclosure or a comparative composition such as commercial products with Product Code “PEB” (Pre-Seed) for 30 minutes is performed as explained in A. Ivic, Hum Reprod 17 (2002): 143-149, hereby incorporated by reference in its entirety, particularly in relation to the use of 2% methylcellulose columns. Briefly, mouse embryo assay and mouse in-vitro fertilization-MEA (F/M strains B6C3F1/B6D2F1) are conducted using established methods with exposure to 10% concentrations of compositions as described herein or the comparative lubricant for 30 minutes. Compatibility of beneficial Lactobacillus crispatus with control media, versus 10% and 50% suspensions of compositions of the present disclosure or the comparative product is determined by mixing the products with bacterial suspension in a Suspension Time-Kill Assay as per established methods such as those described in C S Dezzutti et al., PLoS One 7 (2012): e48328 or M Balouiri et al., J Pharm Anal 6 (2016): 71-79, each hereby incorporated by reference in their entirety particularly in relation to the assays disclosed therein.

Additionally, analyses are performed on normal and abnormal quality semen samples for each of the controls and test groups using JMP Pro 13 (SAS Institute Inc.) or MedCalc statistical software. After testing for normal distribution, data is analyzed using parametric or non-parametric tests including two-way ANOVA test with Tukey's Honestly Significant Difference Test (HDS), or Kruskal-Wallis test. Response variables will include sperm outcomes (e.g., motility, oxidative stress, DNA quality), with at least three replicates, positive and negative controls, and strict pre-set criteria for reproducibility (<10% coefficient of variation), linearity and back-fit analysis of quality control. Relative abundance of bacteria between men with normal and abnormal quality sperm is compared for penile and semen microbiomes (genital microbiomes). Using the software packages such as “vegan,” (e.g., Version 2.5-4 R Foundation for Statistical Computing; 2019) or Primer? (e.g., PRIMER v7, PRIMER-E Plymouth Marine Laboratory, ed2015:20), the online pipeline MicrobiomeAnalyst, community profiling, detection of differentially abundant taxa, and correlation analysis of the genital microbiome is performed as described in D Baud, et al. Front Microbiol (2019): 234, hereby incorporated by reference in its entirety and particularly in relation to analysis of genital microbiome. Microbiota may be broadly classified and positively or negatively correlated with semen quality within subject. Subjects may be sorted by predominant species of genital microbiome (e.g., pathogenic-dominant, beneficial-dominant) similar to as described in ED Borges, et al, J Assist Reprod Genet. 37 (2020): 53-61. Finally, impact of subject's genital microbiome types on sperm function in lubricants is assessed using a two-way ANOVA with post hoc Tukey's HSD for each subject compared across treatments vs. controls.

It is believed that low acidity compositions will have minimal or beneficial effect on gametes as measured in these assays such as a significant change of less than 20% or less than 15% or less 10% or less than 5% as compared to control. In comparison, commercially available products may show demonstrate a significant change (e.g., a change of greater than 20%) as compared to control. All aspects of routine lubricant, mucosal, gamete and microbiota studies will be considered to pass (80% or more of lubricant-free positive controls). Further, a positive, or lack of negative, interaction between sperm function and the different microbiome groups in the treatments of the present disclosure also occurs.

### Example 11: Selective Growth Suppression of Pathobionts

The comparative effect of compositions of the present disclosure on beneficial microbiota (e.g., healthy Lactobacillus species such as L. crispatus, L. jensenii, and L. G. gasseri), species in transitional mucosal environments (e.g., L. inners), and pathobionts such as Gardnerella vaginalis and Prevotella bivia is seen through measurements human in-vitro models of vaginal microbiota colonized human cervical epithelial tissues. Tissues may be exposed to compositions of the present disclosure for a set amount of time for bacterial colonization (e.g., 4 hours) or an amount of time sufficient for biomarker protein expression (e.g., 24 hours). Cell damage and immunotoxicity will be assessed by established, clinically validated biomarkers in parallel with simultaneous assessment of bacterial colonization by: 1) healthy Lactobacillus species (L. crispatus, jensenii & gasseri); 2) Lactobacillus species in dysbiotic or transitional mucosal environments (L. inners); and 3) Pathobionts G. vaginalis (implicated in genesis of bacterial biofilms and dysbiosis) and P. bivia (anaerobe linked to HPV gene expression machinery and metastatic cervical cancer).

The human organotypic model of epithelium used in these studies is comprised of normal primary epithelial cells differentiated in-vitro into polarized multilayered cervicovaginal tissue. Biomarkers (validated in-vivo) will be assessed following contact of bacteria-colonized vaginal epithelia with compositions of the present disclosure compared to placebo (medium) control, proinflammatory control (synthetic TLR2/6 ligand mimicking bacterial and fungal lipoproteins), commercial products RepHresh and Trimosan. Each experiment is repeated at least three times with positive and negative controls, and strict pre-set criteria for meeting standards of reproducibility (<10% coefficient of variation), linearity and back-fit analysis of quality control. All data will be entered in Prism GraphPad for graphic analysis and ordinary one-tailed ANOVA with Tukey multiple comparisons. Control for clustering within experiments will be performed using a two-tailed Rosner-Glynn-Lee Wilcoxon rank-sum test and account for multiple comparisons within bacterial type by a Bonferroni correction.

In vitro models were performed assessing the selective support proffered by Formula H (Table 9) difference between L. crisptatus and P. bivia growth. The measurements on L. crispatus recovery of Formula H as compared to these OTC buffer gel products is shown in Table 48.

TABLE 48
Comparative evaluation of the effects of lead OTC buffer gels on survival
of vaginal bacteria essential for resistance to high-risk HPV

| | L. crispatus recovery: % mean ± SD | | |
|---|---|---|---|
| | No | Pretreatment Compared to | |
| | pretreatment | PreBioGyn | |
| Vaginal Product Brand | 0 h | 4 h | 24 h |
| PreBioGyn | 102.2 ± 1.57 | 100 ± 1.63 | 100 ± 1.00 |
| RepHresh | 100.9 ± 2.18 | 58.67 ± 15.03* | 0* |
| Replens | 104.3 ± 1.66 | 0* | 0* |
| Trimosan | 87.4 ± 0.85* | 0* | 0* |
| McKesson | 48.25 ± 1.18* | 0* | 0* |

*p < 0.0001 lower survival of L. crispatus compared to PreBioGyn Ordinary one-way ANOVA, Tukey multiple comparisons test

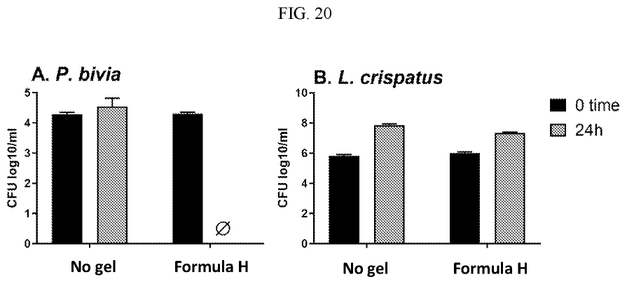

Comparisons of L. cristpatus growth as compared to P. bivia (pathobiont) growth illustrate microbiome optimization afforded by the compositions of the compositions of the present disclosure. FIG. 20 shows the number of CFU of P. bivia as compared to L. crispatus after 24 hours of application to an in vitro model. As can be seen, Formula H selectively suppressed P. bivia while supporting growth of L. crispatus while also demonstrating prebiotic properties similar to standard culture broth (no gel control).

### Example 12: Sensor Measurements

Genital (vaginal and penile) pH is measured with oxidized single-walled carbon nanotubes functionalized with conductive polymer poly(l-aminoanthracene) (PAA) to detect surface pH level by changes in sensor conductance in response to protonation and deprotonation of carboxylic groups of the nanotube and amine groups of the polymer. The sensor is housed in a disposable sheath, integrated with a reusable handheld device, which connects with a purpose-built e-health app for routine genital health status updates, even in users with low healthcare fluency. The integrated e-health app may suggest over the counter (OTC) available management strategies, counseling with remote platform healthcare staff, or sharing of data and referral to user's healthcare providers which may be based on the information received from the sensor. Key novel factors of the sensor system may include: 1) ability to tailor the sensor for specific regional or ethnic groups (one size does not fit all), 2) inclusion of testing for men to improve community-wide reproductive health, and 3) real-time, low-cost assessment of genital health in diffuse, less accessible communities, for development of evidence-based solutions to intransient healthcare challenges.

The carbon nanomaterials to be used in the sensor may sense pH changes in contact with the genital surface as local charge density via functional groups on the PAA are protonated/deprotonated as described in P Gou, et al., Sci Rep 4 (2014): 4468, hereby incorporated by reference in its entirety. These changes alter conductance of the SWCNTs and are then measured via interdigitated electrodes on prefabricated silicon chips made via standard photolithography. An alternative nanocarbon material, holey graphene (HG) as described in DL White, et al., Nano Lett 19 (2019): 2824-2831 and Y Xu et al., Nano Lett 15 (2015): 4605-4610, each of which is hereby incorporated by reference in their entirety and particularly in relation to graphene, holey graphene, and oxidized forms thereof. Briefly, graphene, holey graphene, and oxidized forms thereof achieves similar changes as ox-SWCNT via edge localized moieties including hydroxyl and carboxylic groups and will also be evaluated for the system as described in H Vedala, Nano Lett 11 (2011): 2342-2347, hereby incorporated by reference in its entirety. These carbon nanomaterials are exceedingly sensitive (high surface to volume ratios and superior electrical conductance). Furthermore, carbon nanosensors are advantageous due to high chemical and thermal stability, reversibility of sensing response, high sensitivity, and ability to measure with biosample contact (e.g., inside the vagina, on penis, of genital fluids) as described in D Kauffman et al., Angew Chem Int Ed Engl 47 (2008): 6550-6570, V Schroeder, et al., Chem Rev 119 (2019): 599-663, and M. Meyyappan Small 12 (2016): 2118-2129, each of which is hereby incorporated by reference in their entirety.

The carbon nanomaterial tubes may mitigate the issues of current user-directed pH monitoring, as data can be collected in the biologically relevant environment. in particular, the high chemical stability of nanomaterials is utilized, and may need only initial calibration at fabrication. These sensors may measure resistance changes in the pH sensor, convert the resistance to pH, and display this pH on the prototype screen, either for smartphone camera capture or Bluetooth integration with a purpose-built-health app. Real-time genital symptoms or events may be assessed (new partner, menses, new product) and inform the user of status and any need for medical follow up.

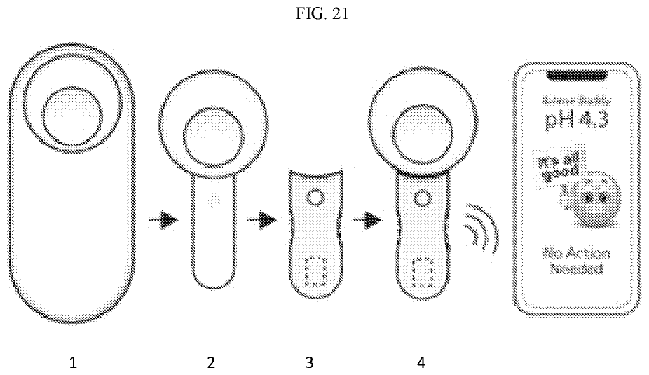

The sensor includes an easy-grip reusable handle with housing. The handle is removed prior to use and a disposable soft sheath containing the single-use nanosensor for pH measurement and metabolomic profiling is snapped on. The sensor may then be placed against a body portion such as the distal vaginal wall or penile surface (at the corona sulcus) for reading via integrated e-health app (e.g., Bluetooth or RFID signaling) which integrates measured outcomes with contextual user symptom/sexual wellness data. The sensor may also comprise a binary and/or ternary signal display system. A sensor of the present including a representation of the application on a smart phone is shown in FIG. 21 where a disposable sheath comprising the sensor unit, after measurement of biological sample from the user, may be removably attached to the handle portion. The handle sensor unit may then transmit the required information to an external device which provides recommendations to the user based on the measured data.

Sensor arrays composed of non-specific chemistry in combination with machine learning algorithms may be used to achieve selectivity through data analytics. This sensor array approach will be used to diagnose bacterial vaginosis. As discussed in G Silva, et al., ACS Sens 2 (2017): 1128-1132, hereby incorporated by reference in its entirety, cell discrimination of carbon-based sensors has been demonstrated by utilizing a non-specific binding array sensor approach utilizing SWCNT FET devices decorated with gold nanoparticles decorated with different self-assembled monolayers (SAMs) (e.g., in FIG. 4). Briefly, cells were immobilized on SWCNT devices and the FET response to the presence of cells was measured. Due to differences in cell membrane composition as well as excreted extracellular material, the response data of the cells studied to identify the unique characteristics of each cell type. These regions were defined by training the device with a large sample size of known identity and applying linear discriminant analysis (LDA) to separate these regions-based sensor responses acquired from each type. By combining analysis of carbon-based FET device characteristics with supervised machine learning algorithms, successfully discrimination among five selected purine compounds was possible. These interactions of purine compounds with metal nanoparticle-decorated SWCNTs were further corroborated by density functional theory (DFT) calculations.

The metabolomic profiles consistent with high-risk genital dysbiosis in men and women in is performed using these techniques.

### Example 13: Muco-Adhesion Potential of the Tested Compositions

Formulation PL1116SSB was prepared with the components as shown in Table 49.

TABLE 49

| | Formula PL1116SSB |
|---|---|
| Ingredient Name | % by wt. |
| Purified Water, USP | 97.872 |
| monosodium phosphate, anhydrous | 0.240 |
| sodium chloride | 0.300 |
| Lactulose | 0.050 |
| (−)-Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Sodium Benzoate | 0.125 |
| Sodium Salicylate | 0.075 |
| Sodium Hydroxide | QS to pH 4.5 |

*The indicated weight percentage of metallic co-factor (i.e., magnesium chloride) was achieved by adding the appropriate amount of a hydrate of the metallic salt (e.g., magnesium chloride (II) tetrahydrate).

Mucoadhesion potential of the formulation and three competitive products was determined in vitro using solutions of natural porcine gastric mucin. Properties and behavior of gastric mucin are similar (if not identical) to mucosa from other regions such as the mucosa of the urogenital and/or anogenital regions. Muco-adhesion occurs when two components, (one being biological in origin), are held together by interfacial forces, including electrical charge and polymer chain entanglement. Muco-adhesive properties may allow better contact of each formulation with the vaginal surface. Superior muco-adhesion potential suggests that the composition is better able to be retained in the vagina, thus aiding in supplementation of vaginal secretions and moisture. Also, if used for drug delivery compositions, those having increased muco-adhesive potential would have superiority with regard to residence time and delivery of an active agent.

The level of muco-adhesion for several compositions was quantified by measuring the change in viscosity due to synergism in a mucin-vaginal moisturizer gel solution as described in D Ivarsson, et al., Colloids and Surfaces B 92 (2012): 353-359, hereby incorporated by reference in its entirety and particularly in relation to the mucin-vaginal moisturizer gel solution. Muco-adhesion strength of compositions was assessed by co-mixing each test composition with 8% mucin from porcine stomach Type II (Sigma-Aldrich). The mucin solution was prepared with 0.9% NaCl solution (saline). The 8% mucin solution was mixed at room temperature with one of the test lubricants (RepHresh™, PL1116SSB Formulation of Table 49, TRIMO-SAN® lubricant, Summer's Eve® douche) to provide final concentrations of 25% (wt/wt) gel/mucin solutions. The RepHresh™ lubricant comprised of purified water USP, glycerin, polycarbophil, Carbopol® 974P, ethylparaben sodium, methylparaben sodium, propylparaben sodium, and sodium hydroxide and had a pH of 3.46. The TRIMO-SAN® lubricant comprised of hydroxyquinoline sulfate 0.025%; sodium lauryl sulfate N.F. 0.01%. adjusted with triethanolamine to ph 4 in water dispersible base composed of glycerine, Carbomer N.F., citric acid, sodium citrate U.S.P., methylparaben N.F., perfume, and antifoam emulsion N.F. The Summer's Eve® comprised of Povidone-Iodine 0.3%, citric acid, edetate disodium, purified water, sodium benzoate, sodium lauryl sulfate, and trisodium phosphate. The relative viscosity of the 8% mucin-only solution was compared to the relative viscosity of the three-resulting mucin-gel solutions by measuring the time required for a 1 mL drop of product/mucin solution to travel 90 mm on an uncoated cardboard surface oriented at a 45° angle. The experiment was performed in duplicate.

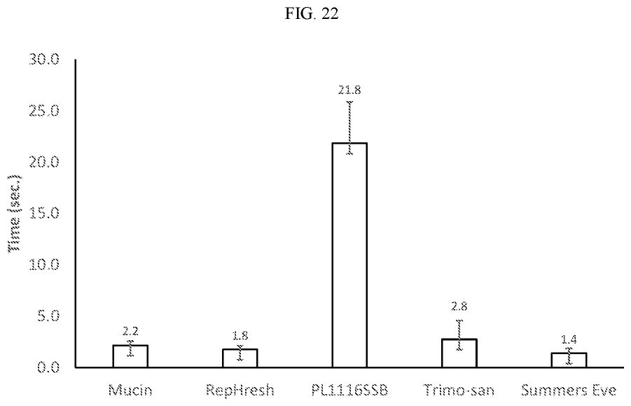

Mucoadhesion differed by gel type. RepHresh™ showed less muco-adhesion, as demonstrated by little change in solution viscosity compared to control (1.8 seconds vs 2.2 seconds). TRIMO-SAN® demonstrated a slight increase in muco-adhesion as shown by a 1.3-fold increase in relative viscosity versus control (2.8 seconds vs 2.2 seconds). Summer's Eve® showed less muco-adhesion, as demonstrated by little change in solution viscosity compared to control (1.4 seconds vs 2.2 seconds). In contrast, the PL1116SSB formulation of the disclosure showed a 10-fold increase in relative viscosity versus control (21.8 seconds versus 2.2 seconds) suggesting a high muco-adhesion potential for the compositions of the disclosure (see FIG. 22). The viscosity differences cannot be explained based on differences in the viscosities of the neat gels.

### Example 14: Perceptual Forearm Gel Studies Associated with Lubricity Positive Attributes of Products During Vaginal Use

Perception of lubricity (“slipperiness”) and tackiness/stickiness as compared to RepHresh™ and GUN OIL® was measured using a published methodology to rate gel attribute on the forearm. See, e.g., Mahan, et al. “Quantitative perceptual differences among over-the-counter vaginal products using a standardized methodology: implications for microbicide development.” Contraception. 84(2):184-193, 2011 (Epub 2011/07/16). The validated methodology included designating three different 4-cm circles on the non-dominant forearm of each participant. The order of gel application and circle to which each gel was applied were randomized. Each treatment was replicated twice. Fingers and circles were wiped with a simple ingredient baby-wipe. The investigator dispensed 0.2 ml of each sample into the center of the designated circle. The sample was then rubbed in a circular direction using the index finger at a rate of 2 rotations per second for 15 seconds followed by another 45 seconds (60 second time point). A metronome set at 120 beats per minute was played to standardize the manipulation rate. Prior to test gel evaluation, participants evaluated an anchor product, GUN OIL® lubricant, that was very slippery and not tacky for reference. GUN OIL® lubricant comprised of cyclopentasiloxane, dimethicone, dimethiconol, tocopherol acetate, and aloe barbadensis leaf extract. Data were analyzed using two-way ANOVA with Tukey's HSD. The degree of slipperiness and tackiness for each product was determined by measuring the distance (in cm) of each participant's mark for different treatments on the VAS, from the left side (not slippery/not tacky) baseline.

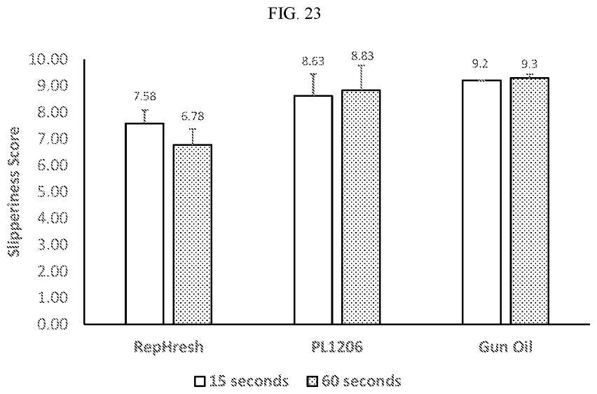

At 15 seconds and 60 seconds of manipulation, perceived slipperiness of each gel was recorded by participants on a 10-cm visual analog scale (VAS). The two ends of the VAS were anchored by the phrases “not slippery” (Slipperiness Score 0) and “very slippery” (Slipperiness Score 10). Participants evaluated RepHresh™, PL1206, and Gun Oil® and found that at both 15 seconds and 60 seconds, the PL1206 formulation was more similar to the Gun Oil® lubricant in terms of slipperiness versus that of RepHresh™. At 15 seconds and 60 seconds, the slipperiness score increased for each gel from RepHresh™ (7.58; 6.78), PL1206 (8.63; 8.83), and Gun Oil® (9.2; 9.3). See, FIG. 23.

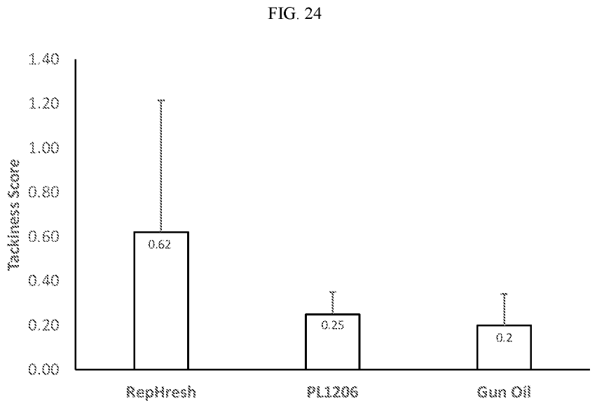

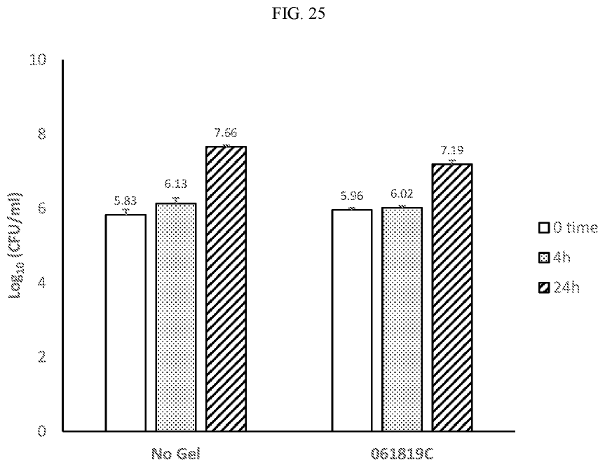

Following the 60 seconds slipperiness evaluation, subjects tapped up and down on the circle of applied gel 6 times to determine tackiness. Tackiness of each gel was recorded by participants on a 10-cm visual analog scale (VAS). See, FIG. 24. The two ends of the VAS were anchored by the phrases “not tacky” (Tackiness score 0) and “very tacky” (Tackiness score 1.4). The tackiness score decreased for each gel from RepHresh™ (0.62), PL1206 (0.25), and Gun Oil® (0.2). See, FIG. 24. The results are provided below and summarized in FIG. 23 (Slipperiness) and FIG. 24 (Tackiness).

FIGS. 23 and 24 show that the PL1206 (also known as 1004BA10 or PL 102345) composition (Table 41) was more slippery at 60 seconds and less tacky or sticky than the existing commercial Class 2 Lubricant RepHresh™ (both p=0.02), and did not differ much from the positive (highly slippery/not tacky) silicone anchor (GUN OIL®). More slippery vaginal products can protect against mucosal microtrauma which commonly occurs during coitus (found in >50% of women within hours of vaginal-penile intercourse versus 11% in abstinent women). See, e.g., Norvell, et al. “Investigation of microtrauma after sexual intercourse.” J Reprod Med. 29(4):269-271, 1984. Epub 1984/04/01. Such mucosal microtrauma can increase risk of inflammation and contracting STDs which lead to cervical cancer (e.g., human papillomavirus (HPV), Herpes simplex virus (HSV), human immunodeficiency virus (HIV)). PL1206 demonstrated better slipperiness and less tackiness as compared to RepHresh™ and would provide better protection against mucosal microtrauma, thereby reducing the risk of inflammation and contracting STDs.

### Example 15: Bactericidal Effects of Formulation 061819C on Lactobacillus Crispatus

Formulation 061819C (Table 31) was tested on Lactobacillus crispatus for bactericidal effects, which is part of vaginal product safety evaluations due to its established role as a keeper of a homeostatic balance in the vaginal mucosa. Priority to L. crispatus was given in the safety algorithm due to its most sensitive responses to potentially toxic compounds which allowed for the best discrimination and ranking in comparison to over-the-counter vaginal products in the model. The effect of a neat gel as well as gels diluted with bacterial culture broth (Peptone Yeast Glucose Tween (PYG TWEEN FA/GLC); Anaerobe Systems Morgan Hill, Calif. 95037) was tested by mixing it with equal parts of L. crispatus suspension at a standardized concentration adjusted to provide a good growth curve within a testing period of 4 hours to 24 hours.

After each desired time period of incubation with the 061819C gel (0 time point, 4 hours, and 24 hours) or no gel, the experimental mix with neat test gel was plated in serial dilutions on Brucella agar and bacteria were assessed for viability by enumeration of colony forming units (CFU) after a 2 to 4 days incubation under anaerobic conditions as described.

This technique resulted in a satisfactory CFU recovery of live L. crispatus from the formulation at all time points and all concentrations. (FIG. 25). Formulation 061819C resulted in similar L. crispatus recovery as without gel over all time points. Without gel at 0 time, 4 hours, and 24 hours, the number of colony forming units per milliliter (CFU/ml) was 5.83, 6.13, and 7.66, respectively, while with Formulation 061819C, the CFU/ml was 5.96, 6.02, and 7.19, respectively. A sample was taken from each of the mixes with the serially diluted gel to assess bacterial growth by a microplate adenosine triphosphate (ATP) assay.

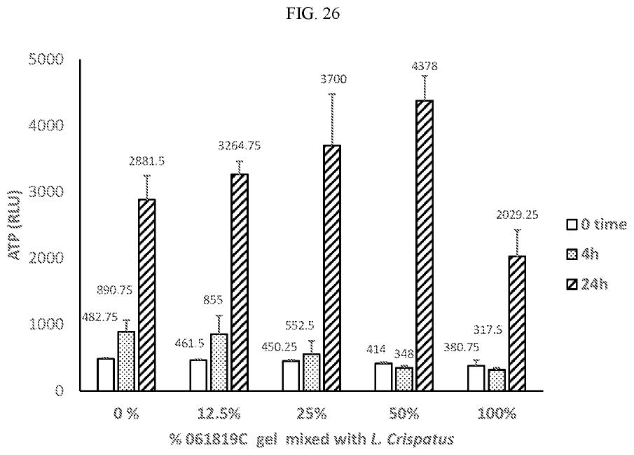

The ATP assay showed that while the neat gel did not fully support bacterial growth over 24 h when displacing the PYG growth medium, dilutions of the gel starting at 2-fold mix with PYG medium were not only able to support but even tended to stimulate the mitochondrial function of L. crispatus suggesting a possible prebiotic activity (FIG. 26). Detection and quantitation of ATP may be used as a means for detecting and/or quantitating microorganisms, such as bacteria, since all living things utilize ATP for storing metabolic energy. FIG. 26 shows the relative light unit (RLU) as a unit of measure for ATP. The sample comprising 50% of the Formulation 061819C gel mixed with Lactobacillus crispatus had optimal ATP or RLU at 24 hours, i.e., over a 10-fold increase compared to 0 time.

### Example 16: Slug Mucosal Irritation (SMI) Assay

The Slug Mucosal Irritation (SMI) assay was developed to predict the mucosal irritation potency of pharmaceutical formulations and ingredients. Briefly, the assay utilized the terrestrial slug Arion lusitanicus, whose body wall is a mucosal surface composed of different layers. The outer single-layered columnar epithelium contains cells with cilia, cells with microvilli, and mucus-secreting cells that cover the subepithelial connective tissue. Slugs placed on an irritating substance produced mucus, while induced tissue damage would result in the release of proteins and enzymes from the mucosal surface. Several studies have shown that the SMI assay is a useful tool for evaluating the local tolerance of pharmaceutical formulations and ingredients. See, e.g., Adrians E and Remon J P. “Gastropods as an evaluation tool for screening the irritating potency of absorption enhancers and drugs.” Pharm. res. 16(8):1240-1244, 1999; Ceulemans J, et al. “Evaluation of a mucoadhesive tablet for ocular use.” JCR. 77(3):333-344, 2001; Callens C, et al. “Toxicological evaluation of a bioadhesive nasal powder containing a starch and Carbopol 974 P on rabbit nasal mucosa and slug mucosa.” JCR. 76(1-3):81-91, 2001; Adriaens E and Remon J P. “Mucosal irritation potential of personal lubricants relates to product osmolality as detected by the slug mucosal irritation assay.” Sex. Transm. Dis. 35(5):512-516, 2008. A classification prediction model that distinguishes between irritation (mucus production) and tissue damage (release of proteins) was developed. See, e.g., Dhondt M M, et al. “The evaluation of the local tolerance of vaginal formulations containing dapivirine using the Slug Mucosal Irritation test and the rabbit vaginal irritation test.” Eur. J. Pharm. Biopharm. 60(3):419-425, 2005.

A study was performed to assess the mucosal irritation potency (MW) of the composition with the Slug Mucosal Irritation (SMI) assay. The mucosal irritation potency of the 1004BA10 composition (also known as PL 102345 or PL 1206) was compared to RepHresh™ lubricant, Summer's Eve® wash, NIVEA MEN® Active Clean wash, and K-Y® Jelly lubricant. The Summer's Eve® wash comprised of water, citric acid, sodium benzoate, disodium EDTA, polysorbate 20, and fragrance. The NIVEA MEN® DEEP Active Clean Body Wash comprised of water, sodium laureth sulfate, cocamidopropyl betaine, acrylates copolymer, PEG-7 glyceryl cocoate, fragrance, charcoal powder, PEG-200 hydrogenated glyceryl palmate, PEG-40 hydrogenated castor oil, PEG-3 distearate, trisodium EDTA, sodium hydroxide, phenoxyethanol, methylparaben, and ethylparaben. The K-Y® Jelly comprised of water, glycerin, hydroxyethylcellulose, gluconolactone, methylparaben, sodium hydroxide, and chlorhexidine digluconate.

The method for testing the irritation potency of the formulations was evaluated by placing the slugs on 100 mg of the sample for 30 minutes daily for 3 successive days, and the amount of mucus produced was measured. Hydroxyethylcellulose (HEC) gel was used as a placebo or negative control. The total mucus production (MP) percentage was calculated as a percent of the slug's body weight. Table 50 shows the tested PL102345 composition had no mucosal irritation potential (MW) and produced less mucus than the negative control (i.e., 2±0.7% vs 4.6±1.1%) even though common douches, personal lubricants, and shower gels are known to cause increased irritation. In fact, each of the tested commercially available douches, body wash, and lubricants produced more mucus than even the nonoxynol-9 positive control. The RepHresh™ lubricant was also tested and found to have a high mucosal irritation potency as shown in FIG. 3 of U.S. Pat. No. 9,919,018, the entire contents of which is incorporated by reference in its entirety. The high and very high mucosal irritation potency of commercially available douches, body washes, and lubricants are associated with vaginal and penile irritation and do harm to beneficial microbiomes.

TABLE 50
Mucosal Irritation Potency (MIP) Results

| Formulation | Total MP (%) | MIP |
|---|---|---|
| Placebo/negative control (HEC gel) | 4.6 ± 1.1 | none |
| Nonoxynol-9/positive control | 8.2 ± 1.2 | high |
| PL102345 | 2.0 ± 0.7 | none |
| Summers Eve ® | 9.6 ± 0.5 | high |
| NIVEA MEN ® Body Wash/shower gel | 10.5 ± 3.4 | high |
| K-Y ® Jelly Lubricant | 20.9 ± 2.3 | very high |

### Example 17: Benefits of Increased Buffering Capacity in Specific Ranges

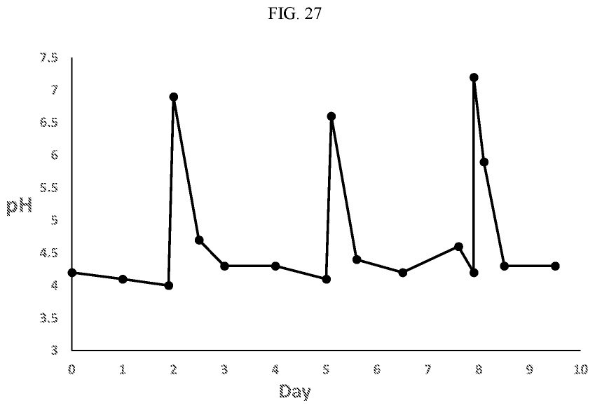

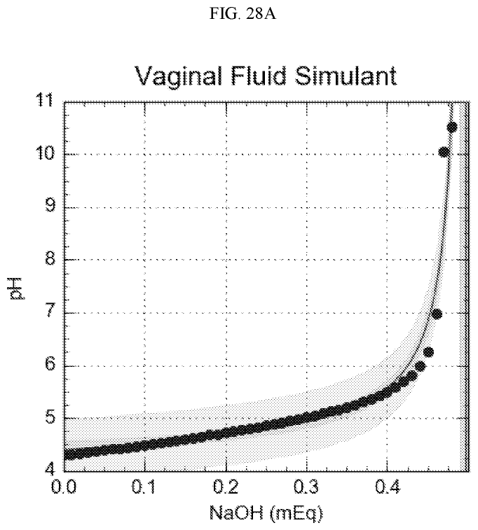

A vaginal fluid simulant (VFS) has been described (Rastogi R, et al. Contraception 93 (2016): 337-346, which is hereby incorporated by reference in its entirety and particularly in relation to the vaginal fluid stimulant composition described therein) and contains pH buffers that mimic the buffering capacity of healthy vaginal fluids. The VFS is able to buffer well in the healthy pH range below 5. However, once pH rises above 5, VFS is quickly overwhelmed, and it is unable to resist further elevations in pH. This allows natural semen buffer systems to protect sperm after ejaculation at the neutral pH they are best optimized at (see FIG. 27). Such excursions in pH during reproduction, also optimally trigger Lactobacillus to enter an active growth curve, with increased lactic acid production returning the vagina to an acidic pH (<4.5) within 12 hrs following intercourse, as a part of normal vaginal biology (lactobacillus asymptotic pH 3.2 to 4.8) as described in Boskey, E., et al. Infect Immun 67 (1999): 5170-5175, hereby incorporated by reference in its entirety.

The relevant buffering capacity of the of vaginal fluid simulant (VFS) and VFS mixed with the prebiotic lubricant of the present disclosure in physiologic quantities were determined. The relevant buffering capacity is the ability of a solution to resist a physiologically meaningful pH shift as described in Rastogi R, et al. Contraception 93 (2016): 337-346 and Cunha A, et al. Pharmaceutics 6 (2014): 530-542, each of which are hereby incorporated by reference in their entirety and particularly in relation to buffering capacity measurements. Briefly, the relevant buffering capacity of the gels when mixed with vaginal fluid simulant (VFS) in physiologic quantities were determined. VFS mimics a woman's vaginal fluids in buffering, ionic composition and pH. In this study the relevant buffering capacity was determined based on the mixtures ability to resist a pH change to values of 5.0, 5.5, 6.0, 6.5, and 7.0, as these elevated levels observed in women with vaginal infections, cancers and the Genitourinary Syndrome of Menopause. Menopause is diagnosed by vaginal pH >5, which often approaches a neutral pH (7) in older women. Mean vaginal pH values over 5 associates with bacterial vaginosis infections. Higher vaginal pH levels between 5 and 7 also associate with increased sexually transmitted disease infections and increased formation and progression of urogenital inflammation, infection and cancers. Higher vaginal pH supports pathogen growth and reduction in protective Lactobacillus dominance of the vagina.

An optimized vaginal microbiome maintains a healthy pH during sexual activity, balancing the protective barrier function needs of the woman and the sperm protective needs for reproduction of the sexual dyad. The healthy acidic/low pH (<4.5) prior to intercourse is elevated by the natural pH of semen immediately after intercourse with ejaculation (seen as pH spikes up to pH ˜7). This higher pH protects sperm and also stimulates growth of healthy vaginal bacteria and their function (e.g. Lactobacillus dominance), which quickly metabolize sugars in the vaginal mucus-hydrogel as an energy substrate and produce lactic acid to lower vaginal pH levels. However, some women do not have adequate Lactobacillus bacteria in the vagina (e.g., lacking Lactobacillus dominance) to restore an acidic pH after the normal vaginal pH increase observed post-coitally. This includes women with infections, menopausal women (pH>5 w.o HRT), and women with urogenital cancers or other diseases. Overall, about 30% of women have had bacterial vaginosis (BV), a dysbiosis associated with pathogen overgrowth in the vagina, with rates as high as 80% in octogenarian women (may be associated with pelvic floor prolapse and chronic urinary tract infections).

BV is caused by a loss of Lactobacillus dominance and an overgrowth of pathobiont or pathogenic bacteria accompanied by an elevation in vaginal pH (>4.5) and an increase in pro-inflammatory mucosal factors. BV associates with higher levels of sexually transmitted infections and urogenital cancers. Many women are asymptomatic for BV and recurrence rates can exceed 50%. Probiotic and prebiotic vaginal care have been proposed to reduce dysbiosis and related diseases. The impact of a composition of a prebiotic lubricant composition of the present disclosure was determined on in vivo vaginal pH and with in vitro vaginal and semen simulants to observe gel buffering potential.

The pH of the VFS, and VFS+a Glyciome prebiotic lubricant (GPL), or lubricant of the present disclosure comprising the combination of bornyl acetate, manganese chloride, and lactulose were measured by direct immersion of a glass pH electrode. The components of the GPL are described in Table 51. Also shown in Table 49 is a lubricant composition of the present disclosure comprising a preservative (sodium dehydroacetate)

TABLE 51

| | No | |
|---|---|---|
| | preservative | Preservative |
| Ingredient Name | (% by Wt.) | (% by Wt.) |
| Purified Water, USP | 97.765 | 97.615 |
| Monosodium Phosphate | 0.240 | 0.240 |
| Sodium Chloride | 0.350 | 0.350 |
| Lactulose | 0.050 | 0.050 |
| Abies Sibirica | 0.020 | 0.020 |
| Hypromellose | 0.600 | 0.600 |
| Gluconolactone | 0.500 | 0.500 |
| Hydroxypropyl Guar | 0.400 | 0.400 |
| Gum | | |
| Arabinogalactan | 0.050 | 0.050 |
| Mentha Spicata | 0.010 | 0.010 |
| Citrus Aurantium Var. | 0.010 | 0.010 |
| Amara | | |
| Manganese Chloride | 0.005 | 0.005 |
| Sodium Dehydroacetate | | 0.15 |
| Sodium Hydroxide | Titrate To pH 4.0 | Titrate To pH 4.0 |

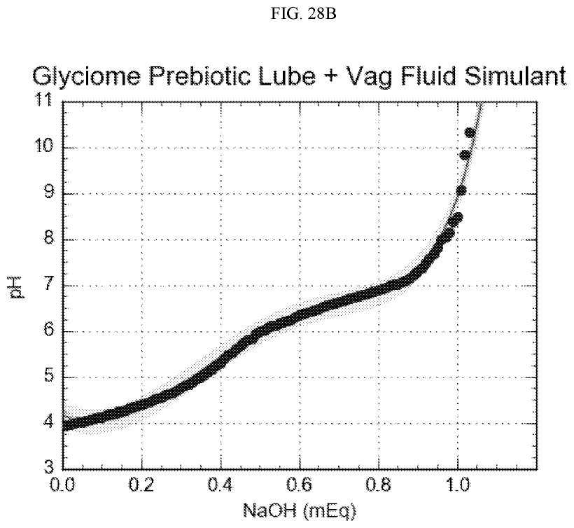

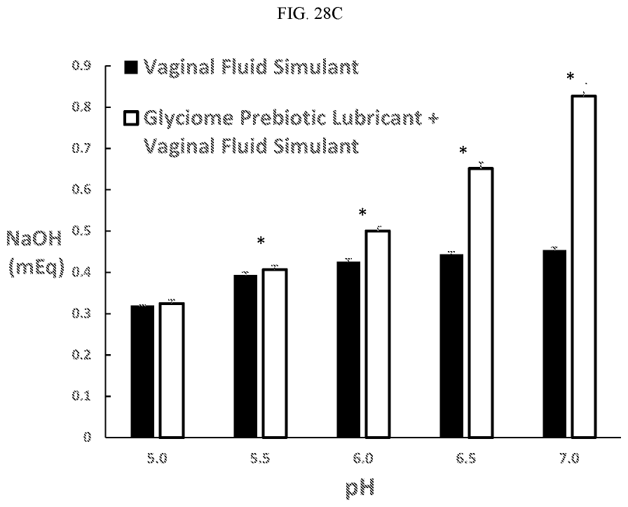

Aliquots (10 ul) of 1 M sodium hydroxide (NaOH) were sequentially added to each solution (the GPL and the VFS) with constant stirring and pH levels recorded in duplicate, until the samples reached pH 10. Titration curves for the buffering capacity were calculated according to the best fit model using CurveExpert Professional Version 2.72 (Copyright 2010-2020, Daniel G. Hyams). This study was replicated three times. FIGS. 28A and 28B are representative buffer curves for the two solutions, showing improved buffering with the GPL vs VFS alone. FIG. 28C represents the mean buffering capacity of each solution at pH ranging from 5 to 7 illustrating improved buffering for the GPL (asterisks indicate difference p<0.05). The improved buffering in this range is the range that vaginal pH normally begins to support pathogenic vaginal microbiota (asymptotic pH for pathogens=4.7 to 6.0). The pH alteration afforded by application of the compositions of the present disclosure are capable to maintain the vaginal microbiome in a range that enhances beneficial microbiome production.

### Example 18: GPL Supports Reproductive Efficiency without Overwhelming Seminal Buffers

Surprisingly, in spite of the improved buffering capability of GPL (pH 4), pre-coital use of the prebiotic lubricant did not overwhelm seminal buffers protective to sperm function and upregulating for healthy lactobacillus growth following intercourse. Use of the GPL prior to coitus also surprisingly supported a more rapid return of the vagina to optimal pH (<4.5) than pre-coital use of either a leading sexual lubricant (Astroglide—pH 4.5) or a fertility lubricant (Pre-Seed—pH 7.1) (p<0.05). Furthermore, the use of the GPL post-coitally (within 10 min after ejaculation), supported an even more rapid restoration of optimal vaginal pH vs other treatments (p<0.05), but did not fully acidify vaginal pH (pH—5.2). The leading sexual lubricant and the fertility lubricant both resulted in vaginal pH levels staying above 4.5 over 24 hrs from use, representing vaginal pH level that support pathogenic bacterial growth (asymptotic pH for pathogens=4.7 to 6.0). The measurements for this analysis are provided below.

Pre-Coital pH was measured no more than 30 min prior to coitus and prior to any lubricant treatment. Immediately following measurement of the pre-coital pH, 3 gms of lubricant (as indicated) were applied vaginally. Coitus was then initiated within 10 min, with ejaculation occurring within another 20 min. Post-Coital pH was measured within 10 min of ejaculation.

For treatments with post-coital application of GPL, no product was applied precoitally. Instead, 3 gms of Glyciome prebiotic lubricant was applied vaginally within 10 min of ejaculation, and subsequent pH measured.

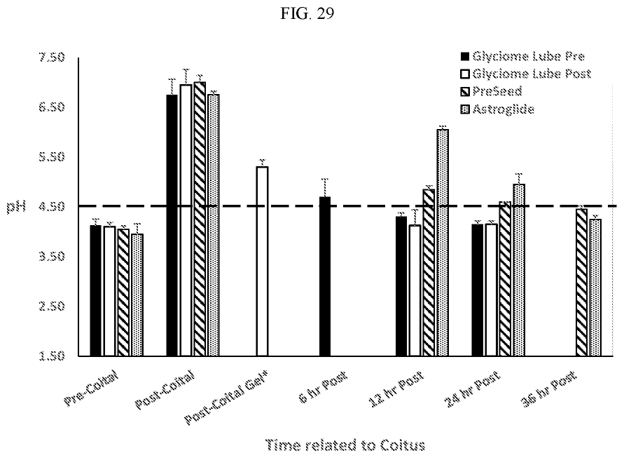

Vaginal pH was measured at 12 hr intervals until vaginal pH was consistently below 4.5 (up to 36 hrs). Precoital GPL treatments were also measured at 6 hrs post coitus. These measurements are illustrated in FIG. 29. The measured treatments were:
Glyciome lubricant pre-coital 3 gm (pH 4; “Glyciome Lube Pre,” Solid Black Bars);
Glyciome lubricant post-coital 3 gm (pH 4; “Glyciome Lube Post,” Solid White Bars);
Pre-Seed lubricant precoital 3 gm (pH 7.1, “PreSeed,” Striped Bars); and
Astroglide lubricant precoital 3 gm (pH 4.5, “Astroglide, Dotted Bars).

As can be seen, GPL supported a more rapid return to a healthy vaginal pH versus commercial lubricants (with a range of starting pH levels). This effect was noted if the prebiotic was used before or after semen deposition in the vagina. Accordingly, the benefits of the presently disclosed compositions are available to larger population segments, since, for example, some women do not need or want to use a precoital lubricant. Furthermore, use precoitally did not overcome healthy semen buffering (keeping the vagina at pH ˜7) protective of sperm function in the vagina for the ˜10-30 min required for sperm transport out of the vagina to the egg while also supporting a more rapid return of the vagina to a healthy pH (within 12 hrs). These results were not seen following use of a commercial fertility lubricant (PreSeed) or a leading sexual lubricant (Astroglide) at (36 hrs).

These findings support the safety of the prebiotic compositions of the present disclosure for use in couples trying to conceive. The novel postcoital use of the prebiotic composition rapidly lowered post coital vaginal pH (5.3) but critically not to the level found to reduce lactobacillus growth optimization (lactobacillus asymptotic pH 3.2 to 4.8).

Contact allergies have been reported in women from semen in the vagina inducing rapid pH changes. These results illustrate that the compositions of the present disclosure could offer relief for these women. Furthermore, prebiotic lubricants as described herein, when used prior to or after seminal ejaculation in the vagina may optimize the healthy vaginal ecology. Homeostasis in the vagina requires that vaginal lactobacilli growth and acidification be gated once optimal vaginal pH is reached. The prebiotic lubricants of the present disclosure are able to support pH levels that are optimal for lactobacillus growth and reacidification of the vagina postcoitally as compared to existing commercial lubricants, without over acidifying the vagina and limiting of healthy lactobacillus growth.

### Example 19: Buffering Capacity of the GPLs are Optimized with the Combinations Present Therein

The in vitro pH buffering capacity of a GPL with a combination of components (bornyl acetate, lactulose, and manganese) as described herein was compared with the same gel formulation without these components and to the same gel formulation with only one of the ingredients (manganese or lactulose) to illustrate the optimization afforded by prebiotic and metallic cofactors in the presently disclosed compositions.

The pH of these gels and VFS mixtures were measured by direct immersion of a glass pH electrode. The weight ratio of VFS to GPL in these measurements was 0.75 g to 2 g (0.375:1), a value used based on an estimated vaginal fluid volume of 0.75 ml in the vagina and the retention of 2 gm gel following a typical intravaginal 3 g dose.

Aliquots (10 μl) of 1 M sodium hydroxide (NaOH) were sequentially added to each solution and pH levels recorded in duplicate until the samples reached pH 8. Measurements were replicated three times. Titration curves for the buffering capacity were calculated according to the best fit model using CurveExpert Professional Version 2.72 (Copyright 2010-2020, Daniel G. Hyams). Multiple comparisons between treatments, concentrations and solutions were analyzed.

The relevant buffering capacity was surprisingly improved for Glyciome gel with the combination (lactulose, manganese, bornyl acetate) as compared to the base gel formula alone, between the ranges of pH 5 to 7. The improvement was most significant at the pH 5.5 to 6 range where elevated pH can support pathogen growth (asymptotic pH for pathogen growth=4.7 to 6.0). Furthermore, the relevant buffering was improved with addition of manganese or lactulose to the base gel formula alone. However, surprisingly neither individual component performed as well as the combination of all three ingredients on buffering capacity in a physiologically meaningful range. Furthermore, bornyl acetate alone did not impact buffering capacity, although its addition in the final formula improved buffering over individual components as shown. The beneficial effect of the novel combination was most noticeable in the pH 5 to 7 ranges, that very range that women with vaginal pathology can benefit from buffering of pH to limit pathogen growth.

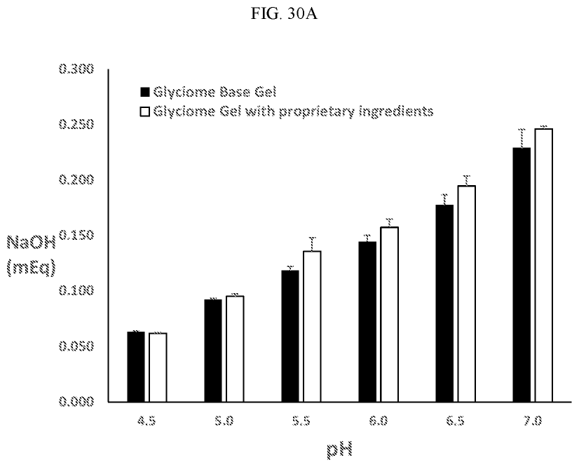

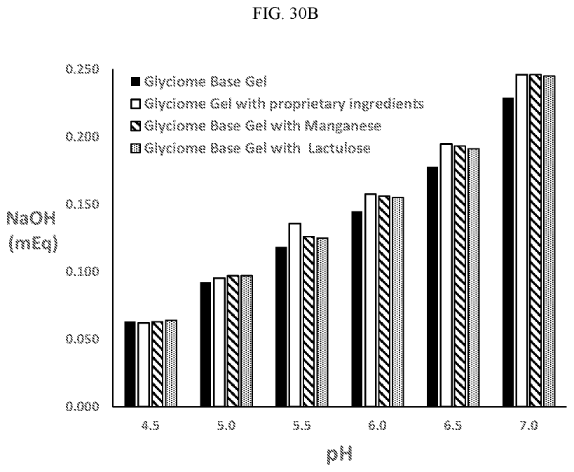

Although manganese has been reported to bind to gluconic acid (the hydrolyzed and proton-donating form of gluconolactone), and would have been predicted to reduce the buffering capacity of the mixture to which it was included, these findings illustrate that the compositions of the present disclosure are operative in spite of such binding (and potential removal of the buffering system by the metallic cofactor present in these compositions). These results are depicted in FIGS. 30A-B where “proprietary ingredients” refers to the combination of bornyl acetate, manganese, and lactulose.

### Example 20: The Effect of Prebiotic Gels of the Present Disclosure (GPG) on pH of Vaginal Fluid Simulant Following Addition of Semen Fluid Simulant (SFS)

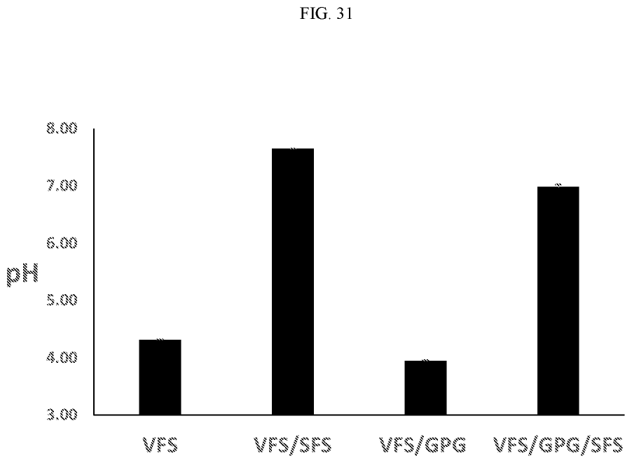

A semen fluid simulant (SFS) has been described that mimics salient pH and buffering properties of human seminal fluid necessary for in vitro product evaluation as described in Rastogi, R. et al., Contraception 93 (2016): 337-346, which is incorporated by reference in its entirety and particularly in relation to semen fluid stimulants. The pH of the GPL, VFS, and SFS mixtures were measured by direct immersion of a glass pH electrode. The weight ratio of VFS:GPL:SFS was 0.75:2:3 (each measured in grams) based on an estimated vaginal fluid volume of 0.75 g in the vagina, the retention of 2 g GPL following a typical intravaginal 3 g dose, and an average 3 g human ejaculate. As shown in FIG. 31, although the GPL does reduce pH of the VFS/SFS mixture, it still allows the pH to rise into the range most protective of sperm function (approximately >6.5). Error bars represent the standard deviation of the measurements performed.

### Example 21: The Effect of the GPL and the Fertility Lubricant Pre-Seed on the Amount of Lactic Acid Necessary to Reduce pH when Added to a Physiologic Combination of a VFS and SFS Mixture

The compositions of the present disclosure were compared to a leading fertility lubricant (Pre-Seed) to identify the amount of lactic acid necessary to increase acidity. The following pH measurements were taken:
a) Control: VFS and SFS in a weight ratio of 0.75:3
b) VFS and SFS with Prebiotic lubricant of the present disclosure in a weight ratio of 0.75:3:2
c) VFS and SFS with Pre-Seed fertility lubricant in a weight ratio ratio of 0.75:3:2
   Pre-seed compositions include water, hydroxyethylcellulose, Pluronic, sodium chloride, sodium phosphate, carbomer, methylparaben, sodium hydroxide, arabinogalactan, potassium phosphate, and propylparaben.

The pH of these mixtures was measured by direct immersion of a glass pH electrode. Aliquots (10 ul) of a 20% lactic acid solution were sequentially added to each solution with constant stirring, and pH levels recorded in duplicate, until the samples reached pH 4 (i.e., a healthy vaginal pH). This study was replicated three times. Titration curves for the buffering capacity were calculated according to the best fit model using CurveExpert Professional Version 2.72 (Copyright 2010-2020, Daniel G. Hyams). The weight ratio of VFS:SFS:lubricant was based on an estimated vaginal fluid volume of 0.75 g in the vagina, an average 3 g ejaculate, and the retention of 2 g lubricant following a typical intravaginal 3 g dose.

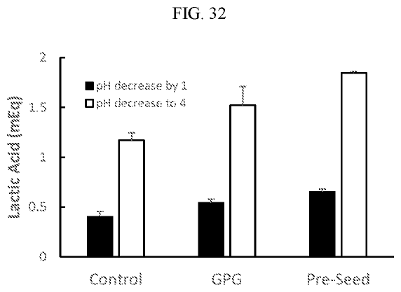

As shown in FIG. 32, the amount of lactic acid necessary to reduce the VFS/SFS combination by a pH of 1 and to a pH of 4 was significantly greater following addition of both lubricants. However, the lubricant of the present disclosure (i.e., containing bornyl acetate, manganese chloride, lactulose, and buffered by a buffer containing gluconolactone) required significantly less lactic acid compared to the Pre-Seed composition. This finding is consistent with the in-vivo findings described in Example 18.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet and Request are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of optimizing the beneficial microbiome growth in the genital region of a subject in need thereof comprising application of a pharmaceutical composition having an acidic pH to the genital region of the subject or to the genital region of a sexual partner of the subject in need thereof.

2. The method according to claim 1, wherein the optimizing the beneficial microbiome growth comprises inhibiting pathogenic bacterial growth and/or promoting beneficial bacterial growth.

3. (canceled)

4. The method according to claim 1, wherein the application has minimal effect to the gametes of the subject or the sexual partner of the subject and/or has minimal effect on zygote or embryo formed after intercourse between the subject and a sexual partner, or during assisted reproduction.

5. The method according to claim 4, wherein the application has minimal effect to the male gametes in any genital fluids secreted from the subject or the sexual partner of the subject.

6. The method according to claim 5, wherein the male gametes have minimal change in their motility and/or concentration and/or vitality and/or morphology and/or oxidation-reduction potential and/or sperm DNA fragmentation and/or sperm mitochondrial membrane potential and/or survival and/or sub-cellular alterations.

7. (canceled)

8. The method according to claim 1, wherein said composition comprises borneol or an ester thereof or a pharmaceutically acceptable salt or prodrug of any of the foregoing and/or a metal cofactor and/or a prebiotic oligosaccharide.

9. The method according to claim 1, wherein said composition comprises an essential oil comprising bornyl acetate.

10-14. (canceled)

15. The method according to claim 1, wherein the composition comprises less than 1% preservative by weight of the composition.

16. A system for the measurement of one or more parameters of the metabolomic profile of a biological sample:
a substrate;
a sensor medium immobilized on said substrate comprising a microfluidic chip and/or a plurality of carbon nanostructures; wherein the plurality of carbon nanostructures have one or more conductive materials deposited thereon;
at least two conductive terminals in electrical connection with the sensor medium and spaced from each other;
at least one measurement system to measure one or more electrical properties of the sensor medium when the sensor medium comprises the biological sample deposited thereon; and
a correlation system calibrated to correlate the measured electrical property with the one or more parameters of the metabolomic profile of the biological sample.

17. The system according to claim 16, wherein the carbon nanostructure is a carbon nanotube or graphene.

18. (canceled)

19. The system according to claim 16, wherein the conductive materials are selected from conductive polymers or a conductive metal or combinations thereof.

20. The system according to claim 16, wherein the conductive material comprises at least two conductive metals in the form of nanoparticles.

21-23. (canceled)

24. The system according to claim 20 wherein one form of metal nanoparticle comprises a self-assembled monolayer of dodecanthiol and another form of metal nanoparticle comprises 11-mercaptounedecanoic acid.

25. The system according to claim 16, wherein the one or more measured electrical properties comprise change in transconductance, threshold voltage shift, relative change in conductance at a specified voltage, change in overall conductance when normalized to the threshold voltage, and the relative change in the minimum conductance, or combinations thereof as compared to the sensor medium not having the biological sample deposited thereon.

26. The system according to claim 16, wherein the substrate is also configured to measure the pH of the biological sample based on the electrical properties of the sensor medium having the biological substance deposited thereon.

27-29. (canceled)

30. The system according to claim 16, wherein the one or more parameters of the metabolomic profile is correlated with the number of one or more species of pathogenic bacteria present in the biological sample, the number of one or more species of beneficial bacteria present in the biological sample, similarity to a metabolomic profile of a biological sample from a healthy individual, similarity to a metabolomic profile of a biological sample from an individual suffering a particular disease, disorder, or condition, or combinations thereof.

31. The system according to claim 16, wherein the metabolomic profile comprises tyramine, cadaverine, agmatine, N-acetylputrescine, carnitine, deoxycarnitine, pipecolic acid, pipecolate, lactate, tyrosine, sphingosine, adenine, guanine, xanthine, uric acid, caffeine, glutamate, phenylalanine, glutathione, glycylproline, or combinations thereof.

32. (canceled)

33. A method of optimizing the beneficial microbiome growth in the genital region of a subject in need thereof comprising:
measurement of one or more parameters of the metabolomic profile of a biological sample with the system according to claim 16;
application of a pharmaceutical composition having an acidic pH to the genital region of the subject or to the genital region of a sexual partner of the subject in need thereof based on the one or more parameters of the metabolomic profile.

34. A device for the measurement of one or more parameters of the metabolomic profile of a biological sample comprising:
a) a handle portion dimensioned to be held in a user's hand comprising a power source; and
b) a sensor portion comprising one or more sensors; wherein the sensors comprise:
a substrate;
a sensor medium immobilized on said substrate comprising a plurality of carbon nanostructures; wherein the plurality of carbon nanostructures has one or more conductive materials deposited thereon;
at least two conductive terminals in electrical connection with the sensor medium and spaced from each other;
wherein said sensor portion is removably attached to the handle portion; and when the sensor portion is attached to the handle portion, the one or more systems are in electrical communication with the power source; and
said device comprises at least one measurement system to measure one or more electrical properties of the sensor medium when the sensor medium comprises the biological sample deposited thereon.

35-49. (canceled)

50. A pharmaceutical composition comprising:
(a) a metallic co-factor;
(b) a prebiotic oligosaccharide;
(c) borneol or a prodrug thereof;
wherein said composition is buffered with a buffer system comprising gluconolactone.
